(19) United States
(12) Patent Application Publication
(10) Pub. No.: US 2014/0271734 A1
(43) Pub. Date: Sep. 18, 2014

(54) BILE ACID RECYCLING INHIBITORS FOR TREATMENT OF BARRETT'S ESOPHAGUS AND GASTROESOPHAGEAL REFLUX DISEASE

(71) Applicant: LUMENA PHARMACEUTICALS, INC., SAN DIEGO, CA (US)

(72) Inventors: Bronislava GEDULIN, Del Mar, CA (US); Michael GREY, Rancho Santa Fe, CA (US); Niall O'DONNELL, Encinitas, CA (US); Bradley T. KELLER, Chesterfield, MO (US)

(21) Appl. No.: 14/213,225

(22) Filed: Mar. 14, 2014

Related U.S. Application Data
(60) Provisional application No. 61/798,876, filed on Mar. 15, 2013.

(51) Int. Cl. A61K 31/5377; A61K 31/495; C07D 295/13; C07D 207/16; C07C 279/12; A61K 31/40; A61K 31/4995; A61K 31/454; A61K 45/06; A61K 31/16; A61K 31/452; C07D 487/08; A61K 31/155

(52) U.S. Cl. CPC A61K 31/5377; C07D 487/08; A61K 31/495; C07D 295/13; C07D 207/16; C07C 279/12; A61K 31/155; A61K 31/4995; A61K 31/454; A61K 45/06; A61K 31/16; A61K 31/452; A61K 31/40
USPC 424/400; 514/237.2; 514/249; 514/252.12; 514/315; 514/326; 514/408; 514/626; 514/635

(57) ABSTRACT

Provided herein are methods of treating or ameliorating Barrett's esophagus by administering to an individual in need thereof a therapeutically effective amount of an Apical Sodium-dependent Bile Acid Transporter Inhibitor (ASBTI) or a pharmaceutically acceptable salt thereof. Provided herein are methods of treating or ameliorating gastroesophageal reflux disease (GERD) by administering to an individual in need thereof a therapeutically effective amount of an ASBTI or a pharmaceutically acceptable salt thereof. Also provided are methods for treating or ameliorating symptoms or complications associated with Barrett's esophagus or GERD comprising administering to an individual in need thereof a therapeutically effective amount of ASBTI or a pharmaceutically acceptable salt thereof.

### CROSS-REFERENCE TO RELATED APPLICATIONS

This application claims the benefit of U.S. Provisional Patent Applicant Ser. No. 61/798,876, filed Mar. 15, 2013, which is herein incorporated by reference in its entirety.

### BACKGROUND OF THE INVENTION

Barrett's esophagus is a disorder in which the lining of the esophagus is damaged by gastroesophageal reflux and changed to a lining similar to that of the stomach or intestine (i.e., intestinal metaplasia). Barrett's esophagus is a serious complication of gastroesophageal reflux disease (GERD), which is a chronic symptom of mucosal damage caused by acid reflux. Barrett's esophagus and GERD share related symptoms. However, patients with Barrett's esophagus have increased risk of developing esophageal adenocarcinoma. Active treatment and prevention is limited.

### SUMMARY OF THE INVENTION

Provided herein are therapeutic compositions and methods for treating or ameliorating Barrett's esophagus and gastroesophageal reflux disease (GERD). In certain embodiments, provided herein are methods for treating or ameliorating Barrett's esophagus comprising non-systemically administering to an individual in need thereof a therapeutically effective amount of an Apical Sodium-dependent Bile Transporter Inhibitor (ASBTI) or a pharmaceutically acceptable salt thereof. In certain embodiments, provided herein are methods for treating or ameliorating Barrett's esophagus comprising administering to an individual in need thereof a therapeutically effective amount of a non-systemically absorbed ASBTI or a pharmaceutically acceptable salt thereof. In certain embodiments, provided herein are methods for treating or ameliorating Barrett's esophagus comprising administering to an individual in need thereof a therapeutically effective amount of a minimally absorbed ASBTI or a pharmaceutically acceptable salt thereof. In certain embodiments, provided herein are methods for treating or ameliorating GERD comprising non-systemically administering to an individual in need thereof a therapeutically effective amount of an Apical Sodium-dependent Bile Transporter Inhibitor (ASBTI) or a pharmaceutically acceptable salt thereof. In certain embodiments, provided herein are methods for treating or ameliorating GERD comprising administering to an individual in need thereof a therapeutically effective amount of a non-systemically absorbed ASBTI or a pharmaceutically acceptable salt thereof. In certain embodiments, provided herein are methods for treating or ameliorating GERD comprising administering to an individual in need thereof a therapeutically effective amount of a minimally absorbed ASBTI or a pharmaceutically acceptable salt thereof.

In some embodiments, provided herein are pharmaceutical compositions for use in the treatment or amelioration of Barrett's esophagus and GERD, wherein the pharmaceutical composition comprises a non-systemically absorbed ASBTI or a pharmaceutically acceptable salt thereof. In some embodiments, provided herein are pharmaceutical compositions for use in the treatment of Barrett's esophagus and GERD, wherein the pharmaceutical composition comprises a non-systemically absorbed ASBTI or a pharmaceutically acceptable salt thereof. In some embodiments, provided herein are pharmaceutical compositions for use in the treatment of Barrett's esophagus, wherein the pharmaceutical composition comprises a non-systemically absorbed ASBTI or a pharmaceutically acceptable salt thereof. In some embodiments, provided herein are pharmaceutical compositions for use in the treatment of GERD, wherein the pharmaceutical composition comprises a non-systemically absorbed ASBTI or a pharmaceutically acceptable salt thereof. In some embodiments, provided herein are pharmaceutical compositions for use in the treatment of Barrett's esophagus and GERD, wherein the pharmaceutical composition consists essentially of a non-systemically absorbed ASBTI or a pharmaceutically acceptable salt thereof. In some embodiments, provided herein are pharmaceutical compositions for use in the treatment of Barrett's esophagus, wherein the pharmaceutical composition consists essentially of a non-systemically absorbed ASBTI or a pharmaceutically acceptable salt thereof. In some embodiments, provided herein are pharmaceutical compositions for use in the treatment of GERD, wherein the pharmaceutical composition consists essentially of a non-systemically absorbed ASBTI or a pharmaceutically acceptable salt thereof.

In certain embodiments, provided herein are methods for increasing GLP-2 levels in a patient suffering from Barrett's esophagus or GERD comprising administering to the patient a therapeutically effective amount of a non-systemically absorbed ASBTI or a pharmaceutically acceptable salt thereof. In certain embodiments, provided herein are methods for decreasing serum or hepatic bile acids in a patient suffering from Barrett's esophagus or GERD comprising administering to the patient a therapeutically effective amount of a non-systemically absorbed ASBTI or a pharmaceutically acceptable salt thereof. In certain embodiments, provided herein are methods for increasing fecal excretion of bile acids in a patient suffering from Barrett's esophagus or GERD comprising administering to the patient a therapeutically effective amount of a non-systemically absorbed ASBTI or a pharmaceutically acceptable salt thereof.

Provided herein are therapeutic compositions and methods for decreasing gastroesphageal reflux of bile acid. In certain embodiments, provided herein are methods for decreasing gastroesphageal reflux of bile acid in a patient suffering from Barrett's esophagus or GERD comprising non-systemically administering to the patient a therapeutically effective amount of an ASBTI or a pharmaceutically acceptable salt thereof. In certain embodiments, provided herein are methods for decreasing gastroesphageal reflux of bile acid comprising administering to an individual in need thereof a therapeutically effective amount of a non-systemically absorbed ASBTI or a pharmaceutically acceptable salt thereof. In certain embodiments, provided herein are methods for decreasing gastroesphageal reflux of bile acid comprising administering to an individual in need thereof a therapeutically effective amount of a minimally absorbed ASBTI or a pharmaceutically acceptable salt thereof.

Provided herein are therapeutic compositions and methods for decreasing the risk of developing esophageal adenocarcinoma in a patient suffering from Barrett's esophagus comprising non-systemically administering to the patient a therapeutically effective amount of an ASBTI or a pharmaceutically acceptable salt thereof. In certain embodiments, provided herein are methods for decreasing the risk of developing esophageal adenocarcinoma in a patient suffering from Barrett's esophagus comprising administering to the patient a therapeutically effective amount of a non-systemically absorbed ASBTI or a pharmaceutically acceptable salt thereof. In certain embodiments, provided herein are methods for decreasing the risk of developing esophageal adenocarcinoma in a patient suffering from Barrett's esophagus comprising administering to the patient a therapeutically effective amount of a minimally absorbed ASBTI or a pharmaceutically acceptable salt thereof.

Provided herein are therapeutic compositions and methods for increasing GLP-2 levels in a patient suffering from Barrett's esophagus or GERD. In certain embodiments, the methods described herein treat or ameliorate Barrett's esophagus or GERD by increasing GLP-2 levels, which is protective of injury caused by Barrett's esophagus or GERD or ameliorate symptoms thereof. In certain embodiments, provided herein are methods for increasing GLP-2 levels or concentrations in a patient suffering from Barrett's esophagus or GERD comprising non-systemically administering to the patient a therapeutically effective amount of an ASBTI or a pharmaceutically acceptable salt thereof. In certain embodiments, provided herein are methods for increasing GLP-2 levels or concentrations comprising administering to an individual in need thereof a therapeutically effective amount of a non-systemically absorbed ASBTI or a pharmaceutically acceptable salt thereof. In certain embodiments, provided herein are methods for increasing GLP-2 levels or concentrations comprising administering to an individual in need thereof a therapeutically effective amount of a minimally absorbed ASBTI or a pharmaceutically acceptable salt thereof. In some embodiments, the methods provided herein increase GLP-2, which reduces necrosis and/or damage to gastroesophageal architecture or reduces instestinal metaplasia.

In some embodiments, compositions and methods provided herein increase GLP-2 levels by at least 100%, 90%, 80%, 70%, 60%, 50%, 45%, 40%, 35%, 30%, 25%, 20%, 15%, or 10%, as compared to the levels prior to administration of the compositions provided herein or as compared to control subjects. In some embodiments, methods provided herein increase GLP-2 levels by at least 100%. In some embodiments, methods provided herein increase GLP-2 levels by at least 90%. In some embodiments, methods provided herein increase GLP-2 levels by at least 80%. In some embodiments, methods provided herein increase GLP-2 levels by at least 70%. In some embodiments, methods provided herein increase GLP-2 levels by at least 60%. In some embodiments, methods provided herein increase GLP-2 levels by at least 50%. In some embodiments, methods provided herein increase GLP-2 levels by at least 40%. In some embodiments, methods provided herein increase GLP-2 levels by at least 30%. In some embodiments, methods provided herein increase GLP-2 levels by at least 25%. In some embodiments, methods provided herein increase GLP-2 levels by at least 20%. In some embodiments, methods provided herein increase GLP-2 levels by at least 15%. In some embodiments, methods provided herein increase GLP-2 levels by at least 10%. In some embodiments, methods provided herein increase GLP-2 levels by at least 5%.

Provided herein are therapeutic compositions and methods for lowering serum bile acid levels or concentrations or hepatic bile acid levels or concentrations in a patient suffering from Barrett's esophagus or GERD. In certain embodiments, provided herein are methods for lowering serum bile acid levels or concentrations or hepatic bile acid levels or concentrations in a patient suffering from Barrett's esophagus or GERD comprising non-systemically administering to the patient a therapeutically effective amount of an ASBTI or a pharmaceutically acceptable salt thereof. In certain embodiments, provided herein are methods for lowering serum bile acid levels or concentrations or hepatic bile acid levels or concentrations comprising administering to an individual in need thereof a therapeutically effective amount of a non-systemically absorbed ASBTI or a pharmaceutically acceptable salt thereof. In certain embodiments, provided herein are methods for lowering serum bile acid levels or concentrations or hepatic bile acid levels or concentrations comprising administering to an individual in need thereof a therapeutically effective amount of a minimally absorbed ASBTI or a pharmaceutically acceptable salt thereof.

In some embodiments, compositions and methods provided herein decrease serum or hepatic bile acid levels by at least 100%, 90%, 80%, 70%, 60%, 50%, 45%, 40%, 35%, 30%, 25%, 20%, 15%, or 10%, as compared to the levels prior to administration of the compositions provided herein or as compared to control subjects. In some embodiments, methods provided herein decrease serum or hepatic bile acid levels by at least 100%. In some embodiments, methods provided herein decrease serum or hepatic bile acid levels by at least 90%. In some embodiments, methods provided herein decrease serum or hepatic bile acid levels by at least 80%. In some embodiments, methods provided herein decrease serum or hepatic bile acid levels by at least 70%. In some embodiments, methods provided herein decrease serum or hepatic bile acid levels by at least 60%. In some embodiments, methods provided herein decrease serum or hepatic bile acid levels by at least 50%. In some embodiments, methods provided herein decrease serum or hepatic bile acid levels by at least 30%. In some embodiments, methods provided herein decrease serum or hepatic bile acid levels by at least 25%. In some embodiments, methods provided herein decrease serum or hepatic bile acid levels by at least 20%. In some embodiments, methods provided herein decrease serum or hepatic bile acid levels by at least 15%. In some embodiments, methods provided herein decrease serum or hepatic bile acid levels by at least 10%. In some embodiments, methods provided herein decrease serum or hepatic bile acid levels by at least 5%.

Provided herein are therapeutic compositions and methods for increasing fecal bile acid excretion in a patient suffering from Barrett's esophagus or GERD. In certain embodiments, provided herein are methods for increasing fecal bile acid levels or concentrations in a patient suffering from Barrett's esophagus or GERD comprising non-systemically administering to the patient a therapeutically effective amount of an ASBTI or a pharmaceutically acceptable salt thereof. In certain embodiments, provided herein are methods for increasing fecal bile acid levels or concentrations comprising administering to an individual in need thereof a therapeutically effective amount of a non-systemically absorbed ASBTI or a pharmaceutically acceptable salt thereof. In certain embodiments, provided herein are methods for increasing fecal bile acid levels or concentrations comprising administering to an individual in need thereof a therapeutically effective amount of a minimally absorbed ASBTI or a pharmaceutically acceptable salt thereof.

In some embodiments, compositions and methods provided herein increase fecal bile acid levels by at least 300%, 250%, 200%, 150%, 100%, 90%, 80%, 70%, 60%, 50%, 45%, 40%, 35%, 30%, 25%, 20%, 15%, or 10%, as compared to the levels prior to administration of the compositions provided herein or as compared to control subjects. In some embodiments, methods provided herein increase fecal bile acid levels by at least 300%. In some embodiments, methods provided herein increase fecal bile acid levels by at least 250%. In some embodiments, methods provided herein increase fecal bile acid levels by at least 200%. In some embodiments, methods provided herein increase fecal bile acid levels by at least 150%. In some embodiments, methods provided herein increase fecal bile acid levels by at least 100%. In some embodiments, methods provided herein increase fecal bile acid levels by at least 90%. In some embodiments, methods provided herein increase fecal bile acid levels by at least 80%. In some embodiments, methods provided herein increase fecal bile acid levels by at least 70%. In some embodiments, methods provided herein increase fecal bile acid levels by at least 60%. In some embodiments, methods provided herein increase fecal bile acid levels by at least 50%. In some embodiments, methods provided herein increase fecal bile acid levels by at least 40%. In some embodiments, methods provided herein increase fecal bile acid levels by at least 30%. In some embodiments, methods provided herein increase fecal bile acid levels by at least 25%. In some embodiments, methods provided herein increase fecal bile acid levels by at least 20%. In some embodiments, methods provided herein increase fecal bile acid levels by at least 15%. In some embodiments, methods provided herein increase fecal bile acid levels by at least 10%. In some embodiments, methods provided herein increase fecal bile acid levels by at least 5%.

Provided herein are therapeutic compositions and methods for reducing intraenterocyte bile acidssalts in a patient suffering from Barrett's esophagus or GERD. In certain embodiments, provided herein are methods for reducing intraenterocyte bile acidssalts in a patient suffering from Barrett's esophagus or GERD comprising non-systemically administering to the patient a therapeutically effective amount of an ASBTI or a pharmaceutically acceptable salt thereof. In certain embodiments, provided herein are methods for reducing intraenterocyte bile acidssalts comprising administering to an individual in need thereof a therapeutically effective amount of a non-systemically absorbed ASBTI or a pharmaceutically acceptable salt thereof. In certain embodiments, provided herein are methods for reducing intraenterocyte bile acidssalts comprising administering to an individual in need thereof a therapeutically effective amount of a minimally absorbed ASBTI or a pharmaceutically acceptable salt thereof.

In certain embodiments, provided herein is an ASBTI or a pharmaceutically acceptable salt thereof for use in the treatment of Barrett's esophagus, wherein the ASBTI is a non-systemically absorbed or is formulated to be non-systemically absorbed. In some embodiments, provided herein is an ASBTI or a pharmaceutically acceptable salt thereof for use in the treatment of GERD, wherein the composition comprises an ASBTI and a pharmaceutically acceptable excipient, wherein the ASBTI is a non-systemically absorbed or is formulated to be non-systemically absorbed. In some embodiments, provided herein is a pharmaceutical composition for use in increasing GLP-2 levels or concentrations in a patient suffering from Barrett's esophagus or GERD, wherein the composition comprises an ASBTI and a pharmaceutically acceptable excipient, wherein the ASBTI is a non-systemically absorbed or is formulated to be non-systemically absorbed. In some embodiments, provided herein is a pharmaceutical composition for use in increasing GLP-2 levels or concentrations in a patient suffering from Barrett's esophagus or GERD, wherein the composition consists essentially of an ASBTI and a pharmaceutically acceptable excipient, wherein the ASBTI is a non-systemically absorbed or is formulated to be non-systemically absorbed. In some embodiments, provided herein is a pharmaceutical composition for use in decreasing serum or hepatic bile acids in a patient suffering from Barrett's esophagus or GERD, wherein the composition comprises an ASBTI and a pharmaceutically acceptable excipient, wherein the ASBTI is a non-systemically absorbed or is formulated to be non-systemically absorbed. In some embodiments, provided herein is a pharmaceutical composition for use in decreasing serum or hepatic bile acids in a patient suffering from Barrett's esophagus or GERD, wherein the composition consists essentially of an ASBTI and a pharmaceutically acceptable excipient, wherein the ASBTI is a non-systemically absorbed or is formulated to be non-systemically absorbed. In some embodiments, provided herein is a pharmaceutical composition for use in increasing fecal excretion of bile acids in a patient suffering from Barrett's esophagus or GERD, wherein the composition comprises an ASBTI and a pharmaceutically acceptable excipient, wherein the ASBTI is a non-systemically absorbed or is formulated to be non-systemically absorbed. In some embodiments, provided herein is a pharmaceutical composition for use in increasing fecal excretion of bile acids in a patient suffering from Barrett's esophagus or GERD, wherein the composition consists essentially of an ASBTI and a pharmaceutically acceptable excipient, wherein the ASBTI is a non-systemically absorbed or is formulated to be non-systemically absorbed.

In certain embodiments, provided herein are compositions comprising a non-systemically absorbed ASBTI or a pharmaceutically acceptable salt thereof. In some embodiments, provided herein are compositions comprising any non-systemically absorbed ASBTI or a pharmaceutically acceptable salt thereof described herein. In some embodiments, provided herein are compositions comprising any minimally absorbed ASBTI or a pharmaceutically acceptable salt thereof described herein. In some embodiments, provided herein are compositions described herein further comprising a second agent described herein. In certain embodiments, provided herein are compositions consisting essentially of a non-systemically absorbed ASBTI or a pharmaceutically acceptable salt thereof. In some embodiments, provided herein are compositions consisting essentially of any non-systemically absorbed ASBTI or a pharmaceutically acceptable salt thereof described herein. In some embodiments, provided herein are compositions consisting essentially of any minimally absorbed ASBTI or a pharmaceutically acceptable salt thereof described herein, and a second agent described herein.

In certain embodiments, described herein are compositions and methods for reducing serum levels of bilirubin, gamma-glutamyl transpeptidase or gamma-glutamyl transferase (GGT), lipase, or liver enzymes, alkaline phosphatase (ALP), alanine aminotransferase (ALT), and aspartate aminotransferase (AST), in an individual in need thereof comprising non-systemically administering to the individual a therapeutically effective amount of an ASBTI or a pharmaceutically acceptable salt thereof.

In certain embodiments, the methods described herein treat or ameliorate Barrett's esophagus or GERD by increasing intestinal intraluminal concentrations of bile acidssalts, which are then excreted in the feces, thereby reducing overall bile acid and serum bile acid or hepatic bile acid load in an individual in need thereof. In certain embodiments, increasing intestinal intraluminal bile acid concentrations according to methods described herein provide protection and/or control of the integrity of an individual's esophagus that has been injured by bile acid reflux of Barrett's esophagus or GERD.

In certain embodiments, the methods described herein treat or ameliorate one or more symptoms of Barrett's esophagus or GERD selected from hematemesis, heartburn, regurgitation, dysphagia, odynophagia, nausea, weight loss, increased salivation, chest pain, reflux esophagitis, esophageal strictures, laryngitis, asthma, sinusitis, pharyngitis, globus pharingeus, globus hystericus, enamel erosion, and dentine hypersensitivity.

In some embodiments, Barrett's esophagus or GERD is pediatric Barrett's esophagus or pediatric GERD. In some embodiments, a patient suffering from Barrett's esophagus or GERD is a pediatric patient. In certain embodiments, the methods described herein treat or ameliorate pediatric Barrett's esophagus or GERD. In some embodiments, any of the methods or compositions described herein reduce gastroesophageal reflux of bile acid in a pediatric patient suffering from Barrett's esophagus or GERD. In some cases, any of the methods or compositions described herein reduce the risk of developing gastroesophageal adenocarcinoma in a pediatric patient suffering from Barrett's esophagus or GERD. In some cases, any of the methods or compositions described herein increase GLP-2 levels or concentrations in a pediatric patient suffering from Barrett's esophagus or GERD. In some cases, any of the methods or compositions described herein lower serum bile acid concentrations or hepatic bile acid concentrations in a pediatric patient suffering from Barrett's esophagus or GERD. In some cases, any of the methods or compositions described herein increase fecal bile acid levels or concentrations in a pediatric patient suffering from Barrett's esophagus or GERD. In some cases, any of the methods or compositions described herein reduce or ameliorate symptoms of Barrett's esophagus or GERD in a pediatric patient.

In some cases, for any of the methods and/or compositions described herein, the individual is an infant less than 2 years of age. In some cases, for any of the methods and/or compositions described herein, the individual is an infant between 0 to 18 months of age. In some cases, for any of the methods and/or compositions described herein, the individual is an infant between 1 to 18 months of age. In some cases, for any of the methods and/or compositions described herein, the individual is an infant between 2 to 18 months of age. In some cases, for any of the methods and/or compositions described herein, the individual is an infant between 3 to 18 months of age. In some cases, for any of the methods and/or compositions described herein, the individual is an infant between 4 to 18 months of age. In some cases, for any of the methods and/or compositions described herein, the individual is an infant between 6 to 18 months of age. In some cases, for any of the methods and/or compositions described herein, the individual is an infant between 18 to 24 months of age. In some cases, for any of the methods and/or compositions described herein, the individual is an infant between 6 to 12 months of age. In some instances, for any of the methods and/or compositions described herein, the individual is a child of between about 2 to about 10 years of age. In some instances, the individual is less than 10 years old. In some instances, the individual is more than 10 years old. In some cases, the individual is an adult.

In certain embodiments, the methods comprise administering a non-systemic ASBTI or an ASBTI formulated to reach the distal gastrointestinal tract. In some embodiments, the distal gastrointestinal tract is jejunum, ileum, colon, or rectum. In some embodiments, the distal gastrointestinal tract is ileum, colon, or the rectum. In some embodiments, the distal gastrointestinal tract is jejunum. In some embodiments, the distal gastrointestinal tract is ileum.

In certain instances, use of the compounds provided herein reduces or inhibits recycling of bile acid salts in the gastrointestinal tract. In some embodiments, the bile transport inhibitors are non-systemic compounds. In some embodiments, the bile transport inhibitors are minimally absorbed compounds. In other embodiments, the bile acid transporter inhibitors are systemic compounds delivered non-systemically. In other embodiments, the bile acid transporter inhibitors are systemic compounds.

In some embodiments of the methods and uses described herein, the ASBTI is a compound of Formula I or a pharmaceutically acceptable salt thereof, as described herein. In some embodiments of the methods and uses described herein, the ASBTI is a compound of Formula II or a pharmaceutically acceptable salt thereof, as described herein. In some embodiments of the methods and uses described herein, the ASBTI is a compound of Formula III or a pharmaceutically acceptable salt thereof, as described herein. In some embodiments of the methods and uses described herein, the ASBTI is a compound of Formula IV or a pharmaceutically acceptable salt thereof, as described herein. In some embodiments of the methods and uses described herein, the ASBTI is a compound of Formula V or a pharmaceutically acceptable salt thereof, as described herein. In some embodiments of the methods and uses described herein, the ASBTI is a compound of Formula VI or Formula VID or a pharmaceutically acceptable salt thereof, as described herein.

In some embodiments, provided herein is a method for treating or ameliorating Barrett's esophagus or GERD comprising non-systemically administering to an individual in need thereof a therapeutically effective amount of an ASBTI of Formula I or a pharmaceutically acceptable salt thereof. In some embodiments, provided herein is a method for reducing gastroesophageal reflux of bile acid comprising non-systemically administering to an individual in need thereof a therapeutically effective amount of an ASBTI of Formula I or a pharmaceutically acceptable salt thereof. In some embodiments, provided herein is a method for decreasing the risk of esophageal adenocarcinoma in a patient suffering from Barrett's esophagus comprising non-systemically administering to the patient a therapeutically effective amount of an ASBTI of Formula I or a pharmaceutically acceptable salt thereof. In some embodiments, provided herein is a method for increasing the levels of GLP-2 in a patient suffering from Barrett's esophagus or GERD comprising non-systemically administering to the patient a therapeutically effective amount of an ASBTI of Formula I or a pharmaceutically acceptable salt thereof. In some embodiments, provided herein is a method for lowering serum bile acid concentrations or hepatic bile acid concentration in a patient suffering from Barrett's esophagus or GERD comprising non-systemically administering to the patient a therapeutically effective amount of an ASBTI of Formula I or a pharmaceutically acceptable salt thereof. In some embodiments, provided herein is a method for increasing fecal bile acids levels in a patient suffering from Barrett's esophagus or GERD comprising non-systemically administering to the patient a therapeutically effective amount of an ASBTI of Formula I or a pharmaceutically acceptable salt thereof.

In some embodiments, provided herein is a method for treating or ameliorating Barrett's esophagus or GERD comprising non-systemically administering to an individual in need thereof a therapeutically effective amount of an ASBTI of Formula II or a pharmaceutically acceptable salt thereof. In some embodiments, provided herein is a method for reducing gastroesophageal reflux of bile acid comprising non-systemically administering to an individual in need thereof a therapeutically effective amount of an ASBTI of Formula II or a pharmaceutically acceptable salt thereof. In some embodiments, provided herein is a method for decreasing the risk of esophageal adenocarcinoma in a patient suffering from Barrett's esophagus comprising non-systemically administering to the patient a therapeutically effective amount of an ASBTI of Formula II or a pharmaceutically acceptable salt thereof. In some embodiments, provided herein is a method for increasing the levels of GLP-2 in a patient suffering from Barrett's esophagus or GERD comprising non-systemically administering to the patient a therapeutically effective amount of an ASBTI of Formula II or a pharmaceutically acceptable salt thereof. In some embodiments, provided herein is a method for lowering serum bile acid concentrations or hepatic bile acid concentration in a patient suffering from Barrett's esophagus or GERD comprising non-systemically administering to the patient a therapeutically effective amount of an ASBTI of Formula II or a pharmaceutically acceptable salt thereof. In some embodiments, provided herein is a method for increasing fecal bile acids levels in a patient suffering from Barrett's esophagus or GERD comprising non-systemically administering to the patient a therapeutically effective amount of an ASBTI of Formula II or a pharmaceutically acceptable salt thereof.

In some embodiments, provided herein is a method for treating or ameliorating Barrett's esophagus or GERD comprising non-systemically administering to an individual in need thereof a therapeutically effective amount of an ASBTI of Formula III or a pharmaceutically acceptable salt thereof. In some embodiments, provided herein is a method for reducing gastroesophageal reflux of bile acid comprising non-systemically administering to an individual in need thereof a therapeutically effective amount of an ASBTI of Formula III or a pharmaceutically acceptable salt thereof. In some embodiments, provided herein is a method for decreasing the risk of esophageal adenocarcinoma in a patient suffering from Barrett's esophagus comprising non-systemically administering to the patient a therapeutically effective amount of an ASBTI of Formula III or a pharmaceutically acceptable salt thereof. In some embodiments, provided herein is a method for increasing the levels of GLP-2 in a patient suffering from Barrett's esophagus or GERD comprising non-systemically administering to the patient a therapeutically effective amount of an ASBTI of Formula III or a pharmaceutically acceptable salt thereof. In some embodiments, provided herein is a method for lowering serum bile acid concentrations or hepatic bile acid concentration in a patient suffering from Barrett's esophagus or GERD comprising non-systemically administering to the patient a therapeutically effective amount of an ASBTI of Formula III or a pharmaceutically acceptable salt thereof. In some embodiments, provided herein is a method for increasing fecal bile acids levels in a patient suffering from Barrett's esophagus or GERD comprising non-systemically administering to the patient a therapeutically effective amount of an ASBTI of Formula III or a pharmaceutically acceptable salt thereof.

In some embodiments, provided herein is a method for treating or ameliorating Barrett's esophagus or GERD comprising non-systemically administering to an individual in need thereof a therapeutically effective amount of an ASBTI of Formula IV or a pharmaceutically acceptable salt thereof. In some embodiments, provided herein is a method for reducing gastroesophageal reflux of bile acid comprising non-systemically administering to an individual in need thereof a therapeutically effective amount of an ASBTI of Formula IV or a pharmaceutically acceptable salt thereof. In some embodiments, provided herein is a method for decreasing the risk of esophageal adenocarcinoma in a patient suffering from Barrett's esophagus comprising non-systemically administering to the patient a therapeutically effective amount of an ASBTI of Formula IV or a pharmaceutically acceptable salt thereof. In some embodiments, provided herein is a method for increasing the levels of GLP-2 in a patient suffering from Barrett's esophagus or GERD comprising non-systemically administering to the patient a therapeutically effective amount of an ASBTI of Formula IV or a pharmaceutically acceptable salt thereof. In some embodiments, provided herein is a method for lowering serum bile acid concentrations or hepatic bile acid concentration in a patient suffering from Barrett's esophagus or GERD comprising non-systemically administering to the patient a therapeutically effective amount of an ASBTI of Formula IV or a pharmaceutically acceptable salt thereof. In some embodiments, provided herein is a method for increasing fecal bile acids levels in a patient suffering from Barrett's esophagus or GERD comprising non-systemically administering to the patient a therapeutically effective amount of an ASBTI of Formula IV or a pharmaceutically acceptable salt thereof.

In some embodiments, provided herein is a method for treating or ameliorating Barrett's esophagus or GERD comprising non-systemically administering to an individual in need thereof a therapeutically effective amount of an ASBTI of Formula V or a pharmaceutically acceptable salt thereof. In some embodiments, provided herein is a method for reducing gastroesophageal reflux of bile acid comprising non-systemically administering to an individual in need thereof a therapeutically effective amount of an ASBTI of Formula V or a pharmaceutically acceptable salt thereof. In some embodiments, provided herein is a method for decreasing the risk of esophageal adenocarcinoma in a patient suffering from Barrett's esophagus comprising non-systemically administering to the patient a therapeutically effective amount of an ASBTI of Formula V or a pharmaceutically acceptable salt thereof. In some embodiments, provided herein is a method for increasing the levels of GLP-2 in a patient suffering from Barrett's esophagus or GERD comprising non-systemically administering to the patient a therapeutically effective amount of an ASBTI of Formula V or a pharmaceutically acceptable salt thereof. In some embodiments, provided herein is a method for lowering serum bile acid concentrations or hepatic bile acid concentration in a patient suffering from Barrett's esophagus or GERD comprising non-systemically administering to the patient a therapeutically effective amount of an ASBTI of Formula V or a pharmaceutically acceptable salt thereof. In some embodiments, provided herein is a method for increasing fecal bile acids levels in a patient suffering from Barrett's esophagus or GERD comprising non-systemically administering to the patient a therapeutically effective amount of an ASBTI of Formula V or a pharmaceutically acceptable salt thereof.

In some embodiments, provided herein is a method for treating or ameliorating Barrett's esophagus or GERD comprising non-systemically administering to an individual in need thereof a therapeutically effective amount of an ASBTI of Formula VI or a pharmaceutically acceptable salt thereof. In some embodiments, provided herein is a method for reducing gastroesophageal reflux of bile acid comprising non-systemically administering to an individual in need thereof a therapeutically effective amount of an ASBTI of Formula VI or a pharmaceutically acceptable salt thereof. In some embodiments, provided herein is a method for decreasing the risk of esophageal adenocarcinoma in a patient suffering from Barrett's esophagus comprising non-systemically administering to the patient a therapeutically effective amount of an ASBTI of Formula VI or a pharmaceutically acceptable salt thereof. In some embodiments, provided herein is a method for increasing the levels of GLP-2 in a patient suffering from Barrett's esophagus or GERD comprising non-systemically administering to the patient a therapeutically effective amount of an ASBTI of Formula VI or a pharmaceutically acceptable salt thereof. In some embodiments, provided herein is a method for lowering serum bile acid concentrations or hepatic bile acid concentration in a patient suffering from Barrett's esophagus or GERD comprising non-systemically administering to the patient a therapeutically effective amount of an ASBTI of Formula VI or a pharmaceutically acceptable salt thereof. In some embodiments, provided herein is a method for increasing fecal bile acids levels in a patient suffering from Barrett's esophagus or GERD comprising non-systemically administering to the patient a therapeutically effective amount of an ASBTI of Formula VI or a pharmaceutically acceptable salt thereof.

In certain embodiments, an ASBTI is any compound described herein that inhibits recycling of bile acidssalts in the gastrointestinal tract of an individual. In certain embodiments, an ASBTI is (−)-(3R,5R)-trans-3-butyl-3-ethyl-2,3,4,5-tetrahydro-7,8-dimethoxy-5-phenyl-1,4-benzothiazepine1,1-dioxide; (“Compound 100A”) or any other salt or analog thereof. In certain of any of the aforementioned embodiments, an ASBTI is 1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]4-aza-1-azoniabicyclo[2.2.2]octane methane sulfonate salt (“Compound 100B”) or any other salt or analog thereof. In certain embodiments, an ASBTI is N, N-dimethylimido-dicarbonimidic diamide (“Compound 100C”) or any salt or analog thereof. In certain embodiments, an ASBTI is any commercially available ASBTI including but not limited to LUM001, LUM002, A-3309, 264W94, S-8921, BARI-1741, HMR-1453, TA-7552, R-146224, or SC-435. In some embodiments, an ASBTI is 1,1-dioxo-3,3-dibutyl-5-phenyl-7-methylthio-8-(N-{(R)-α-[N—((R)-1-carboxy-2-methylthio-ethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxybutyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((R)-1-carboxy-2-methylthioethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-[N-{(R)-α-carboxy4-hydroxybenzyl}carbamoylmethoxy]-2,3,4,5-tetrahydro-1,2,5-benzothiadiazepine; or 1,1-dioxo-3,3-dibutyl-5-phenyl-7-methylthio-8-(N-{(R)-α-[N-(carboxymethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′4 (S)-1-carboxyethyl)carbamoyl]benzyl}carbamoylmethoxy)-2,3,4,5-tetrahydro-1,5-benzothiazepine; or a pharmaceutically acceptable salt thereof; 1-[[5-[[3-[(3S,4R,5R)-3-butyl-7-(dimethylamino)-3-ethyl-2,3,4,5-tetrahydro-4-hydroxy-1,1-dioxido-1-benzothiepin-5yl]phenyl]amino]-5-oxopentyl]amino]-1-deoxy-D-glucitol; or Potassium((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl)sulphate ethanolate, hydrate. In certain embodiments, an ASBTI is 264W94 (Glaxo), SAR548304 (Sanofi), SC-435 (Pfizer), SD-5613 (Pfizer), or A3309 (Astra-Zeneca).

In some embodiments, an ASBTI is not 1,1-dioxo-3,3-dibutyl-5-phenyl-7-methylthio-8-(N-{(R)-α-[N—((R)-1-carboxy-2-methylthio-ethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxybutyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((R)-1-carboxy-2-methylthioethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-[N-{(R)-α-carboxy4-hydroxybenzyl}carbamoylmethoxy]-2,3,4,5-tetrahydro-1,2,5-benzothiadiazepine; or 1,1-dioxo-3,3-dibutyl-5-phenyl-7-methylthio-8-(N-{(R)-α-[N-(carboxymethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N′-((S)-1-carboxyethyl)carbamoyl]benzyl}carbamoylmethoxy)-2,3,4,5-tetrahydro-1,5-benzothiazepine; or a pharmaceutically acceptable salt thereof.

In certain embodiments, methods provided herein further comprise administration of a second agent selected from ursodiol, UDCA, cholestyramineresins, antihistamine agents (e.g., hydroxyzine, diphenhydramine), rifampin, naloxone, Phenobarbital, dronabinol (CB1 agonist), methotrexate, corticosteroids, cyclosporine, colchicines, TPGS—vitamin A, D, E, or K optionally with polyethylene glycol, zinc, and a resin or sequestrant for absorbing bile acids or an analog thereof. In certain embodiments, methods provided herein further comprise administration of a second agent selected from a bile acid or salt with reduced toxicity or a hydrophilic bile acid such as ursodiol, norursodiol, ursodeoxycholic acid, chenodeoxycholic acid, cholic acid, taurocholic acid, ursocholic acid, glycocholic acid, glycodeoxycholic acid, taurodeoxycholic acid, taurocholate, glycochenodeoxycholic acid, or tauroursodeoxycholic acid.

In some embodiments, the dosage of an ASBTI is between about 1 μg/kg/day and about 10 mg/kg/day. In some embodiments, the dosage of an ASBTI is between about 5 μg/kg/day and about 1 mg/kg/day. In some embodiments, the dosage of an ASBTI is between about 10 μg/kg/day and about 300 μg/kg/day. In some embodiments, the dosage of an ASBTI is any dosage from about 14 μg/kg/day and about 280 μg/kg/day. In some embodiments, the dosage of an ASBTI is any dosage from about 14 μg/kg/day and about 140 μg/kg/day. In some embodiments, the dosage of an ASBTI is between about 5 μg/kg/day and about 200 μg/kg/day. In some embodiments, the dosage of an ASBTI is between about 10 μg/kg/day and about 200 μg/kg/day. In some embodiments, the dosage of an ASBTI is between about 10 μg/kg/day and about 175 μg/kg/day. In some embodiments, the dosage of an ASBTI is between about 10 μg/kg/day and about 150 μg/kg/day. In some embodiments, the dosage of an ASBTI is between about 10 μg/kg/day and about 140 μg/kg/day. In some embodiments, the dosage of an ASBTI is between about 25 μg/kg/day and about 140 μg/kg/day. In some embodiments, the dosage of an ASBTI is between about 50 μg/kg/day and about 140 μg/kg/day. In some embodiments, the dosage of an ASBTI is between about 70 μg/kg/day and about 140 μg/kg/day. In some embodiments, the dosage of an ASBTI is between about 10 μg/kg/day and about 100 μg/kg/day. In some embodiments, the dosage of an ASBTI is 10 μg/kg/day. In some embodiments, the dosage of an ASBTI is 20 μg/kg/day. In some embodiments, the dosage of an ASBTI is 30 μg/kg/day. In some embodiments, the dosage of an ASBTI is 35 μg/kg/day. In some embodiments, the dosage of an ASBTI is 40 μg/kg/day. In some embodiments, the dosage of an ASBTI is 50 μg/kg/day. In some embodiments, the dosage of an ASBTI is 60 μg/kg/day. In some embodiments, the dosage of an ASBTI is 70 μg/kg/day. In some embodiments, the dosage of an ASBTI is 80 μg/kg/day. In some embodiments, the dosage of an ASBTI is 90 μg/kg/day. In some embodiments, the dosage of an ASBTI is 100 μg/kg/day. In some embodiments, the dosage of an ASBTI is 110 μg/kg/day. In some embodiments, the dosage of an ASBTI is 120 μg/kg/day. In some embodiments, the dosage of an ASBTI is 130 μg/kg/day. In some embodiments, the dosage of an ASBTI is 140 μg/kg/day. In some embodiments, the dosage of an ASBTI is 150 μg/kg/day. In some embodiments, the dosage of an ASBTI is 175 μg/kg/day.

In some embodiments, provided herein are dosages of an ASBTI between 14 μg/kg/day and 140 μg/kg/day, or between 14 μg/kg/day and 280 μg/kg/day.

In some embodiments, the dosage of an ASBTI is between about 0.1 mg/day and about 50 mg/day. In some embodiments, the dosage of an ASBTI is between about 0.5 mg/day and about 50 mg/day. In some embodiments, the dosage of an ASBTI is between about 0.5 mg/day and about 40 mg/day. In some embodiments, the dosage of an ASBTI is between about 0.5 mg/day and about 30 mg/day. In some embodiments, the dosage of an ASBTI is between about 1 mg/day and about 20 mg/day. In some embodiments, the dosage of an ASBTI is between about 1 mg/day and about 10 mg/day. In some embodiments, the dosage of an ASBTI is between about 1 mg/day and about 5 mg/day. In some embodiments, the dosage of an ASBTI is 1 mg/day. In some embodiments, the dosage of an ASBTI is 5 mg/day. In some embodiments, the dosage of an ASBTI is 10 mg/day. In some embodiments, the dosage of an ASBTI is 20 mg/day. In some embodiments, the dosage of an ASBTI is between 0.5 mg/day and 5 mg/day. In some embodiments, the dosage of an ASBTI is between 0.5 mg/day and 4.5 mg/day. In some embodiments, the dosage of an ASBTI is between 0.5 mg/day and 4 mg/day. In some embodiments, the dosage of an ASBTI is between 0.5 mg/day and 3.5 mg/day. In some embodiments, the dosage of an ASBTI is between 0.5 mg/day and 3 mg/day. In some embodiments, the dosage of an ASBTI is between 0.5 mg/day and 2.5 mg/day. In some embodiments, the dosage of an ASBTI is between 0.5 mg/day and 2 mg/day. In some embodiments, the dosage of an ASBTI is between 0.5 mg/day and 1.5 mg/day. In some embodiments, the dosage of an ASBTI is between 0.5 mg/day and 1 mg/day. In some embodiments, the dosage of an ASBTI is between 1 mg/day and 4.5 mg/day. In some embodiments, the dosage of an ASBTI is between 1 mg/day and 4 mg/day. In some embodiments, the dosage of an ASBTI is between 1 mg/day and 3.5 mg/day. In some embodiments, the dosage of an ASBTI is between 1 mg/day and 3 mg/day. In some embodiments, the dosage of an ASBTI is between 1 mg/day and 2.5 mg/day. In some embodiments, the dosage of an ASBTI is between 1 mg/day and 2 mg/day. In some embodiments, the dosage of an ASBTI is 0.5 mg/day. In some embodiments, the dosage of an ASBTI is 1 mg/day. In some embodiments, the dosage of an ASBTI is 1.5 mg/day. In some embodiments, the dosage of an ASBTI is 2 mg/day. In some embodiments, the dosage of an ASBTI is 2.5 mg/day. In some embodiments, the dosage of an ASBTI is 3 mg/day. In some embodiments, the dosage of an ASBTI is 3.5 mg/day. In some embodiments, the dosage of an ASBTI is 4 mg/day. In some embodiments, the dosage of an ASBTI is 4.5 mg/day. In some embodiments, the dosage of an ASBTI is 5 mg/day. In some embodiments, the pediatric dosage described herein is the dosage of the total composition administered.

In some embodiments, the dosage form comprises 0.5 mg of the ASBTI. In some embodiments, the dosage form comprises 1 mg of the ASBTI. In some embodiments, the dosage form comprises 2.5 mg of the ASBTI. In some embodiments, the dosage form comprises 5 mg of the ASBTI. In some embodiments, the dosage form comprises 10 mg of the ASBTI. In some embodiments, the dosage form comprises 20 mg of the ASBTI.

In certain embodiments, the dosage of an ASBTI is given once a day. In some embodiments, the dosage of an ASBTI is given q.d. In some embodiments, the dosage of an ASBTI is given once a day in the morning. In some embodiments, the dosage of an ASBTI is given once a day at noon. In some embodiments, the dosage of an ASBTI is given once a day in the evening or night. In some embodiments, the dosage of an ASBTI is given twice a day. In some embodiments, the dosage of an ASBTI is given b.i.d. In some embodiments, the dosage of an ASBTI is given twice a day, in the morning and noon. In some embodiments, the dosage of an ASBTI is given twice a day, in the morning and evening. In some embodiments, the dosage of an ASBTI is given twice a day, in the morning and night. In some embodiments, the dosage of an ASBTI is given twice a day, at noon and in the evening. In some embodiments, the dosage of an ASBTI is given twice a day, at noon and in the night. In some embodiments, the dosage of an ASBTI is given three times a day. In some embodiments, the dosage of an ASBTI is given t.i.d. In some embodiments, the dosage of an ASBTI is given four times a day. In some embodiments, the dosage of an ASBTI is given q.i.d. In some embodiments, the dosage of an ASBTI is given every four hours. In some embodiments, the dosage of an ASBTI is given q.q.h. In some embodiments, the dosage of an ASBTI is given every other day. In some embodiments, the dosage of an ASBTI is given q.o.d. In some embodiments, the dosage of an ASBTI is given three times a week. In some embodiments, the dosage of an ASBTI is given t.i.w.

In some embodiments, the dosage form comprises 0.5 mg of the ASBTI given once a day in the a.m. In some embodiments, the dosage form comprises 0.5 mg of the ASBTI given once a day in the p.m. In some embodiments, the dosage form comprises 0.5 mg of the ASBTI given twice a day in the a.m. and the p.m. In some embodiments, the dosage form comprises 1 mg of the ASBTI given once a day in the a.m. In some embodiments, the dosage form comprises 1 mg of the ASBTI given once a day in the p.m. In some embodiments, the dosage form comprises 1 mg of the ASBTI given twice a day in the a.m. and the p.m. In some embodiments, the dosage form comprises 2.5 mg of the ASBTI given once a day in the a.m. In some embodiments, the dosage form comprises 2.5 mg of the ASBTI given once a day in the p.m. In some embodiments, the dosage form comprises 2.5 mg of the ASBTI given twice a day in the a.m. and the p.m. In some embodiments, the dosage form comprises 5 mg of the ASBTI given once a day in the a.m. In some embodiments, the dosage form comprises 5 mg of the ASBTI given once a day in the p.m. In some embodiments, the dosage form comprises 5 mg of the ASBTI given twice a day in the a.m. and the p.m. In some embodiments, the dosage form comprises 10 mg of the ASBTI given once a day in the a.m. In some embodiments, the dosage form comprises 10 mg of the ASBTI given once a day in the p.m. In some embodiments, the dosage form comprises 10 mg of the ASBTI given twice a day in the a.m. and the p.m. In some embodiments, the dosage form comprises 20 mg of the ASBTI given once a day in the a.m. In some embodiments, the dosage form comprises 20 mg of the ASBTI given once a day in the p.m. In some embodiments, the dosage form comprises 20 mg of the ASBTI given twice a day in the a.m. and the p.m.

Provided in certain embodiments herein are methods and dosage forms (e.g., oral or rectal dosage form) for use in the treatment of Barrett's esophagus or GERD, or for use in lowering serum bile acid or hepatic bile acid levels in a patient suffering from Barrett's esophagus or GERD comprising a therapeutically effective amount of an ASBTI, or a pharmaceutically acceptable salt thereof, and a carrier. In some embodiments, methods comprise orally administering a therapeutically effective amount of a minimally absorbed ASBTI, or a pharmaceutically acceptable salt thereof, to an individual in need thereof. In some embodiments, methods comprise rectally administering a therapeutically effective amount of a minimally absorbed ASBTI, or a pharmaceutically acceptable salt thereof, to an individual in need thereof. In specific embodiments, the dosage form is an enteric formulation, an ileal-pH sensitive release formulation, or a suppository or other suitable form.

In some embodiments, a composition for use as described herein comprises at least one of a spreading agent or a wetting agent. In some embodiments, the composition comprises an absorption inhibitor. In some cases an absorption inhibitor is a mucoadhesive agent (e.g., a mucoadhesive polymer). In certain embodiments, the mucoadhesive agent is selected from methyl cellulose, polycarbophil, polyvinylpyrrolidone, sodium carboxymethyl cellulose, and combinations thereof. In some embodiments, the enteroendocrine peptide secretion enhancing agent is covalently linked to the absorption inhibitor. In certain embodiments, the pharmaceutical composition comprises an enteric coating. In some embodiments, a composition for use as described herein comprises a carrier. In certain embodiments, the carrier is a rectally suitable carrier. In certain embodiments, any pharmaceutical composition described herein is formulated as a suppository, an enema solution, a rectal foam, or a rectal gel. In some embodiments, any pharmaceutical composition described herein comprises an orally suitable carrier.

In some embodiments, provided herein is a pharmaceutical composition formulated for non-systemic ileal, rectal or colonic delivery of the ASBTI.

In some embodiments, the methods described herein further comprise administration of a second agent selected from a proton pump inhibitor, an H₂ antagonist, an H₂ receptor inhibitor, an antacid, a prokinetic, alginic acid, sucralfate, baclofen, and a combination thereof.

In some embodiments, the ASBTI is administered orally. In some embodiments, the ASBTI is administered as an ileal-pH sensitive release formulation that delivers the ASBTI to the distal ileum, colon and/or rectum of an individual. In some embodiments, the ASBTI is administered as an enterically coated formulation. In some embodiments, oral delivery of an ASBTI provided herein can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action (the ileum) by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

In some embodiments of the methods described herein, the ASBTI is administered before ingestion of food. In some embodiments of the methods described herein, the ASBTI is administered with or after ingestion of food.

In some embodiments, the methods provided herein further comprise administration of vitamin supplements to compensate for reduced digestion of vitamins, in particular fat-soluble vitamins, in an individual with a condition described herein. In some embodiments, the vitamin supplements comprise fat-soluble vitamins. In some embodiments, the fat-soluble vitamins are vitamin A, D, E, or K.

In some embodiments, the methods and compositions provided herein further comprise administration of a bile acid sequestrant or binder for reducing gastrointestinal side effects. In some embodiments, methods comprise administering a labile bile acid sequestrant, wherein the labile bile acid sequestrant has a low affinity in the colon or rectum of the individual for at least one bile acid. In some embodiments, a labile bile acid sequestrant provided herein releases a bile acid in the colon or the rectum of a human. In some embodiments, a labile bile acid sequestrant provided herein does not sequester a bile acid for excretion or elimination in feces. In some embodiments, a labile bile acid sequestrant provided herein is a non-systemic labile bile acid sequestrant. In some embodiments, non-systemic labile bile acid sequestrant is less than 1%, 2%, 3%, 4%, 5%, 6%, 7%, 8%, 9%, 10%, 15%, 20%, 25%, 30%, 35%, 40%, 45% absorbed systemically. In some embodiments, the labile bile acid sequestrant is lignin or a modified lignin. In some embodiments, the labile bile acid sequestrant is a polycationic polymer or copolymer. In certain embodiments, the labile bile acid sequestrant is a polymer or copolymer comprising one or more N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; cholestyramine, colestipol, or colesevelamor a combination thereof.

In some embodiments, the methods provided herein further comprise partial external biliary diversion (PEBD).

Provided in some embodiments herein is a kit comprising any composition described herein (e.g., a pharmaceutical composition formulated for rectal administration) and a device for localized delivery within the rectum or colon. In certain embodiments, the device is a syringe, bag, or a pressurized container.

### BRIEF DESCRIPTION OF THE DRAWINGS

FIG. 1 illustrates the change in fecal bile acid excretion in ZDF rats after oral administration of 264W94.

FIG. 2 illustrates the change in plasma bile acid concentrations in ZDF rats after oral administration of 264W94 or LUM002.

FIGS. 3A and 3B illustrate an animal efficacy study on oral dose of LUM001 compared to cholestyramine on serum bile acids in dogs.

FIG. 4 illustrates an animal efficacy study on oral dose of LUM001 on fecal bile acids in rats.

FIG. 5 illustrates a serum bile acid (SBA) analysis of healthy subjects after administration of ascending multiple oral doses of LUM001 in a randomized, double-blind, placebo-controlled study.

FIG. 6 illustrates fecal bile acid analysis of healthy subjects after administration of ascending multiple oral doses of LUM001 in a randomized, double-blind, placebo-controlled study.

FIG. 7 illustrates fasting serum bile acid levels and morning post-prandial peak in children under the age of 12 who were administered LUM001 (QD).

FIG. 8 illustrates an animal efficacy study on oral dose of LUM002 on fecal bile acids in hamsters.

FIGS. 9A and 9B illustrate 24-hour fecal bile acid concentrations in ZDF rats after oral administration of LUM002 or SC-435.

FIGS. 10A and 10B illustrate plasma total serum bile acids in ZDF rats after oral administration of LUM002 or SC-435.

FIGS. 11A and 11B illustrate changes in ALP in ZDF rats after oral administration of LUM002 or SC-435.

FIGS. 12A and 12B illustrate changes in ASAT in ZDF rats after oral administration of LUM002 or SC-435.

FIG. 13 illustrates changes in ALAT in ZDF rats after oral administration of LUM002 or SC-435.

FIG. 14 illustrates levels of plasma triglycerides in ZDF rats after oral administration of LUM002 or SC-435.

FIGS. 15A and 15B illustrate levels of baseline-corrected percent Hemoglobin Ale (HbA1c) in ZDF rats after oral administration of LUM002 or SC-435.

FIGS. 16A and 16B illustrate levels of GLP-2 in plasma in ZDF rats after oral administration of LUM002 or SC-435.

FIG. 17 illustrates levels of plasma lipase in ZDF rats after oral administration of LUM002 or SC-435.

FIGS. 18A and 18B illustrate levels of plasma amylase in ZDF rats after oral administration of LUM002 or SC-435.

### DETAILED DESCRIPTION OF THE INVENTION

Bile acidssalts play a critical role in activating digestive enzymes and solubilizing fats and fat-soluble vitamins and are involved in liver, biliary, and intestinal disease. Bile acids are synthesized in the liver by a multistep, multiorganelle pathway. Hydroxyl groups are added to specific sites on the steroid structure, the double bond of the cholesterol B ring is reduced and the hydrocarbon chain is shortened by three carbon atoms resulting in a carboxyl group at the end of the chain. The most common bile acids are cholic acid and chenodeoxycholic acid (the “primary bile acids”). Before exiting the hepatocytes and forming bile, the bile acids are conjugated to either glycine (to produce glycocholic acid or glycochenodeoxycholic acid) or taurine (to produce taurocholic acid or taurochenodeoxycholic acid). The conjugated bile acids are called bile salts and their amphipathic nature makes them more efficient detergents than bile acids. Bile salts, not bile acids, are found in bile.

Bile salts are excreted by the hepatocytes into the canaliculi to form bile. The canaliculi drain into the right and left hepatic ducts and the bile flows to the gallbladder. Bile is released from the gallbladder and travels to the duodenum, where it contributes to the metabolism and degradation of fat. The bile salts are reabsorbed in the terminal ileum and transported back to the liver via the portal vein. Bile salts often undergo multiple enterohepatic circulations before being excreted via feces. A small percentage of bile salts may be reabsorbed in the proximal intestine by either passive or carrier-mediated transport processes. Most bile salts are reclaimed in the distal ileum by a sodium-dependent apically located bile acid transporter referred to as apical sodium-dependent bile acid transporter (ASBT). At the basolateral surface of the enterocyte, a truncated version of ASBT is involved in vectorial transfer of bile acidssalts into the portal circulation. Completion of the enterohepatic circulation occurs at the basolateral surface of the hepatocyte by a transport process that is primarily mediated by a sodium-dependent bile acid transporter. Intestinal bile acid transport plays a key role in the enterohepatic circulation of bile salts. Molecular analysis of this process has recently led to important advances in our understanding of the biology, physiology and pathophysiology of intestinal bile acid transport.

Within the intestinal lumen, bile acid concentrations vary, with the bulk of the reuptake occurring in the distal intestine. Bile acidssalts alter the growth of bacterial flora in the gut. Described herein are certain compositions and methods that control bile acid concentrations in the intestinal lumen, thereby controlling the hepatocellular damage caused by bile acid accumulation in the liver.

Barrett's esophagus is a disorder in which the lining of the esophagus is damaged by gastroesophageal reflux and changed to a lining similar to that of the stomach or intestine (i.e., intestinal metaplasia). Barrett's esophagus is a serious complication of gastroesophageal reflux disease (GERD), which is a chronic symptom of mucosal damage caused by acid reflux. Barrett's esophagus and GERD share related symptoms. However, Barrett's esophagus increases the risk of developing esophageal adenocarcinoma.

In some aspects, the compositions and methods provided herein increase bile acid concentrations in the gut. The increased concentrations of bile acidssalts stimulate subsequent secretion of factors that protect and control integrity of the intestine when it is injured by Barrett's esophagus or GERD.

In another aspect, the compositions and methods described herein have an advantage over systemically absorbed agents. The compositions and methods described herein utilize ASBT inhibitors that are not systemically absorbed. Thus the compositions are effective without leaving the gut lumen, thereby reducing any toxicity and/or side effects associated with systemic absorption.

In a further aspect, the compositions and methods described herein stimulate the release of GLP-2 or other enteroendocrine hormones (e.g., PYY, GLP-1). Increased secretion of GLP-2 allows for prevention or treatment of Barrett's esophagus or GERD by controlling the adaptive process, attenuating intestinal injury, reducing bacterial translocation, inhibiting the release of free radical oxygen, inhibiting production of proinflammatory cytokines, or any combination thereof.

Described herein is the use of inhibitors of the ASBT or any recuperative bile salt transporter that are active in the gastrointestinal (GI) tract for treating or ameliorating Barrett's esophagus or GERD in an individual in need thereof. In certain embodiments, described herein is the use of inhibitors of the ASBT or any recuperative bile salt transporter that are active in the gastrointestinal (GI) tract for increasing GLP-2 levels or concentrations in a patient suffering from Barrett's esophagus or GERD. In certain embodiments, described herein is the use of inhibitors of the ASBT or any recuperative bile salt transporter that are active in the gastrointestinal (GI) tract for decreasing serum or hepatic bile acids in a patient suffering from Barrett's esophagus or GERD. In certain embodiments, described herein is the use of inhibitors of the ASBT or any recuperative bile salt transporter that are active in the gastrointestinal (GI) tract for increasing fecal excretion of bile acids in a patient suffering from Barrett's esophagus or GERD. In certain embodiments, described herein is the use of inhibitors of the ASBT or any recuperative bile salt transporter that are active in the gastrointestinal (GI) tract for decreasing gastroesphageal reflux of bile acid in a patient suffering from Barrett's esophagus or GERD. In certain embodiments, described herein is the use of inhibitors of the ASBT or any recuperative bile salt transporter that are active in the gastrointestinal (GI) tract for decreasing the risk of developing esophageal adenocarcinoma in a patient suffering from Barrett's esophagus or GERD.

In certain embodiments, the methods provided herein comprise administering a therapeutically effective amount of an ASBT inhibitor (ASBTI) to an individual in need thereof. In some embodiments, such ASBT inhibitors are not systemically absorbed. In some of such embodiments, such bile salt transport inhibitors include a moiety or group that prevents, reduces or inhibits the systemic absorption of the compound in vivo. In some embodiments, a charged moiety or group on the compounds prevents, reduces or inhibits the compounds from leaving the gastrointestinal tract and reduces the risk of side effects due to systemic absorption. In some other embodiments, such ASBT inhibitors are systemically absorbed. In some embodiments, the ASBTI provided herein are formulated for non-systemic delivery to the distal ileum. In some embodiments, an ASBTI is minimally absorbed. In some embodiments, an ASBTI is non-systemically administered to the colon or the rectum of an individual in need thereof.

In some embodiments, such ASBT inhibitors are not systemically absorbed. In some of such embodiments, such bile salt transport inhibitors include a moiety or group that prevents, reduces or inhibits the systemic absorption of the compound in vivo. In some embodiments, a charged moiety or group on the compounds prevents, reduces or inhibits the compounds from leaving the gastrointestinal tract and reduces the risk of side effects due to systemic absorption. In some other embodiments, such ASBT inhibitors are systemically absorbed. In some embodiments, the ASBTI are formulated for non-systemic delivery to the distal ileum. In some embodiments, an ASBTI is minimally absorbed. In some embodiments, an ASBTI is non-systemically administered to the colon or the rectum of an individual in need thereof.

Non-systemic ASBTIs as a class of drugs and exemplary species are described in the art. For example, Curr. Med. Chem. 13:997-1016 describes such non-systemicnon-absorbable ASBTIs (aka BARI) including various exemplary species. Non-systemic ASBTIs are not limited to certain structures, but are diverse in structure. Non-systemic absorption property of ASBTI can be predicted via Lipinski's “Rule of 5”, which is a principle in medicinal chemistry for determining non-systemic absorption of compounds based on molecular properties. Lipinski et al., 2001, Adv. Drug Delivery Rev. 46:3-26 describes that non-systemic absorption results from one or more factors: (1) there are more than 5 H-bond donors; (2) the molecular weight is over 500; (3) the Log P is over 5; (4) there are more than 10 H-bond acceptors; (5) compound class that are substrates for biological transporters are exceptions to the rule.

In some embodiments, less than 50%, less than 40%, less than 30%, less than 20%, less than 10%, less than 9%, less than 8%, less than 7%, less than 6%, less than 5%, less than 4%, less than 3%, less than 2%, or less than 1% of the ASBTI is systemically absorbed. In certain embodiments, ASBTIs described herein inhibit scavenging of bile salts by recuperative bile acid salt transporters in the distal gastrointestinal tract (e.g., the distal ileum, the colon and/or the rectum).

In some instances, the inhibition of bile salt recycling results in higher concentrations of bile salts in the lumen of the distal gastrointestinal tract or portions thereof (e.g., the distal small bowel and/or colon and/or rectum). As used herein, the distal gastrointestinal tract includes the region from the distal ileum to the anus. In some embodiments, the compounds described herein reduce intraenterocyte bile acidssalts or accumulation thereof. In some embodiments, the compounds described herein reduce damage to hepatocellular or intestinal architecture associated with Barrett's esophagus or GERD.

### Mammalian Microbiome, Bile Acid Pools and Metabolic Interactions

The integrated metabolism of the bile acid pools in the intestinal lumen lends itself to complex biochemical interactions between host and microbiome symbionts.

Bile acidssalts are synthesized from cholesterol in the liver by a multi-enzyme coordinated process and are crucial for the absorption of dietary fats and lipid-soluble vitamins in the intestine. Bile acidssalts play a role in maintaining the intestinal barrier function to prevent intestinal bacterial overgrowth and translocation, as well as invasion of underlying tissues by enteric bacteria.

Under normal conditions (i.e., when an individual is not suffering from Barrett's esophagus or GERD), symbiotic gut microorganisms (microbiome) interact closely with the host's metabolism and are important determinants of health. Many bacterial species in the gut are capable of modifying and metabolizing bile acidssalts and the gut flora affects systemic processes such as metabolism and inflammation.

Bile acidssalts have strong antimicrobial and antiviral effects—deficiency leads to bacterial overgrowth and increased deconjugation, leading to less ileal resorption. In animals, conjugated bile acid feeding abolishes bacterial overgrowth, decreases bacterial translocation to lymph nodes and reduces endotoxemia.

Accordingly, the methods and compositions described herein allow for replacement, displacement, and/or redirection of bile acidssalts to different areas of the gastrointestinal tract thereby affecting (e g, inhibiting or slowing) growth of microorganisms that may cause infection-associated with Barrett's esophagus or GERD.

Provided herein are methods and compositions for stimulating epithelial proliferation and/or regeneration of intestinal lining and/or enhancement of the adaptive processes in the intestine in individuals with Barrett's esophagus or GERD. In some of such embodiments, the methods comprise increasing bile acid concentrations and/or GLP-2 concentrations in the intestinal lumen.

Increased levels of bile acids, and elevated levels of AP (alkaline phosphatase), alanine aminotransferase (ALT), and aspartate aminotransferase (AST), LAP (leukocyte alkaline phosphatase), gamma GT (gamma-glutamyl transpeptidase), and 5′-nucleotidase are biochemical hallmarks of Barrett's esophagus or GERD. Accordingly, provided herein are methods and compositions for stimulating epithelial proliferation and/or regeneration of intestinal lining and/or enhancement of the adaptive processes in the intestine in individuals with elevated levels of AP (alkaline phosphatase), alanine aminotransferase (ALT), and aspartate aminotransferase (AST), LAP (leukocyte alkaline phosphatase), gamma GT (gamma-glutamyl transpeptidase or GGT), and/or 5′-nucleotidase. In some of such embodiments, the methods comprise increasing bile acid concentrations in the intestinal lumen. Further provided herein, are methods and compositions for reducing elevated levels of AP (alkaline phosphatase), alanine aminotransferase (ALT), and aspartate aminotransferase (AST), LAP (leukocyte alkaline phosphatase), gamma GT (gamma-glutamyl transpeptidase), and 5′-nucleotidase comprising reducing overall bile acid load by excreting bile acid in the feces.

Another symptom of Barrett's esophagus or GERD is the increase in serum concentration of conjugated bilirubin. Elevated serum concentrations of conjugated bilirubin result in jaundice and dark urine. The magnitude of elevation is not diagnostically important as no relationship has been established between serum levels of conjugated bilirubin and the severity of Barrett's esophagus or GERD. Conjugated bilirubin concentration rarely exceeds 30 mg/dL. Accordingly, provided herein are methods and compositions for stimulating epithelial proliferation and/or regeneration of intestinal lining and/or enhancement of the adaptive processes in the intestine in individuals with elevated serum concentrations of conjugated bilirubin. In some of such embodiments, the methods comprise increasing bile acid concentrations in the intestinal lumen. Further provided herein, are methods and compositions for treating elevated serum concentrations of conjugated bilirubin comprising reducing overall bile acid load by excreting bile acid in the feces.

Increased serum concentration of nonconjugated bilirubin is also considered diagnostic of Barrett's esophagus or GERD. Portions of serum bilirubin and covalently bound to albumin (delta bilirubin or biliprotein). This fraction may account for a large proportion of total bilirubin in patients with jaundice. The presence of large quantities of delta bilirubin indicates long-standing Barrett's esophagus or GERD. Delta bilirubin in cord blood or the blood of a newborn is indicative of Barrett's esophagus or GERD that antedates birth. Accordingly, provided herein are methods and compositions for stimulating epithelial proliferation and/or regeneration of intestinal lining and/or enhancement of the adaptive processes in the intestine in individuals with elevated serum concentrations of nonconjugated bilirubin or delta bilirubin. In some of such embodiments, the methods comprise increasing bile acid concentrations in the intestinal lumen. Further provided herein, are methods and compositions for treating elevated serum concentrations of nonconjugated bilirubinand delta bilirubin comprising reducing overall bile acid load by excreting bile acid in the feces.

Barrett's esophagus or GERD results in bile reflux. Bile salts are regurgitated from the hepatocyte into the serum, which results in an increase in the concentration of bile salts in the peripheral circulation. Furthermore, the uptake of bile salts entering the liver in portal vein blood is inefficient, which results in spillage of bile salts into the peripheral circulation. Accordingly, provided herein are methods and compositions for stimulating epithelial proliferation and/or regeneration of intestinal lining and/or enhancement of the adaptive processes in the intestine in patients. In some of such embodiments, the methods comprise increasing bile acid concentrations in the intestinal lumen. Further provided herein, are methods and compositions for reducing overall bile acid load by excreting bile acid in the feces.

Serum cholesterol is elevated in Barrett's esophagus or GERD due to the decrease in circulating bile salts which contribute to the metabolism and degradation of cholesterol. Cholesterol retention is associated with an increase in membrane cholesterol content and a reduction in membrane fluidity and membrane function. Furthermore, as bile salts are the metabolic products of cholesterol, the reduction in cholesterol metabolism results in a decrease in bile acidsalt synthesis. Serum cholesterol observed in children with Barrett's esophagus or GERD ranges between about 1,000 mg/dL and about 4,000 mg/dL. Accordingly, provided herein are methods and compositions for stimulating epithelial proliferation and/or regeneration of intestinal lining and/or enhancement of the adaptive processes in the intestine in individuals with hyperlipidemia. In some of such embodiments, the methods comprise increasing bile acid concentrations in the intestinal lumen. Further provided herein, are methods and compositions for treating hyperlipidemia comprising reducing overall bile acid load by excreting bile acid in the feces.

In children with chronic Barrett's esophagus or GERD, one of the major consequences is failure to thrive. Failure to thrive is a consequence of reduced delivery of bile salts to the intestine, which contributes to inefficient digestion and absorption of fats, and reduced uptake of vitamins (vitamins E, D, K, and A are all malabsorbed in Barrett's esophagus or GERD). Furthermore, the delivery of fat into the colon can result in colonic secretion and diarrhea. Treatment of failure to thrive involves dietary substitution and supplementation with long-chain triglycerides, medium-chain triglycerides, and vitamins. Accordingly, provided herein are methods and compositions for stimulating epithelial proliferation and/or regeneration of intestinal lining and/or enhancement of the adaptive processes in the intestine in individuals (e.g., children) with failure to thrive. In some of such embodiments, the methods comprise increasing bile acid concentrations in the intestinal lumen. Further provided herein, are methods and compositions for treating failure to thrive comprising reducing overall bile acid load by excreting bile acid in the feces.

In some embodiments, any of the methods disclosed herein further comprise administration of an additional active agent selected from: choleretic agents (e.g., ursodiol), phenobarbitols, corticosteroids (e.g., prednisone and budesonide), immunosuppressive agents (e.g., azathioprine, cyclosporine A, methotrexate, chlorambucil and mycophenolate), sulindac, bezafibrate, tamoxifen, lamivudine, and combinations thereof. In some embodiments, the methods are used to treat individuals that are non-responsive to treatment with choleretic agents (e.g., ursodiol), phenobarbitols, corticosteroids (e.g., prednisone and budesonide), immunosuppressive agents (e.g., azathioprine, cyclosporine A, methotrexate, chlorambucil and mycophenolate), sulindac, bezafibrate, tamoxifen, lamivudine, and combinations thereof. In some embodiments, the methods are used to treat individuals that are non-responsive to treatment with choleretic agents. In some embodiments, the methods are used to treat individuals that are non-responsive to treatment with ursodiol.

### Compounds

In some embodiments, provided herein are ASBT inhibitors that reduce or inhibit bile acid recycling in the distal gastrointestinal (GI) tract, including the distal ileum, the colon and/or the rectum. In certain embodiments, the ASBTIs are systemically absorbed. In certain embodiments, the ASBTIs are not systemically absorbed. In some embodiments, ASBTIs described herein are modified or substituted (e.g., with a -L-K group or other non-systemic moiety) to be non-systemic. In certain embodiments, any ASBT inhibitor is modified or substituted with one or more charged groups (e.g., K) and optionally, one or more linker (e.g., L), wherein L and K are as defined herein.

In some embodiments, an ASBTI suitable for the methods described herein is a compound of Formula I:

[chemical structure]

wherein:
R¹ is a straight chained C₁₋₆ alkyl group;
R² is a straight chained C₁₋₆ alkyl group;
R³ is hydrogen or a group OR¹¹ in which R¹¹ is hydrogen, optionally substituted C₁₋₆ alkyl or a C₁₋₆ alkylcarbonyl group;
R⁴ is pyridyl or optionally substituted phenyl or -L_{z}-K_{z}; wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
R⁵, R⁶, R⁷ and R⁸ are the same or different and each is selected from hydrogen, halogen, cyano, R⁵-acetylide, OR¹⁵, optionally substituted C₁₋₆ alkyl, COR¹⁵, CH(OH)R¹⁵, S(O)ₙR¹⁵, P(O)(OR¹⁵)₂, OCOR¹⁵, OCF3, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)ₙR¹⁵, O(CH₂)ₚSO₃R¹⁵, O(CH₂)ₚNR¹²R¹³, O(CH₂)ₚN⁺R¹²R¹³R¹⁴ and —W—R³¹, wherein W is O or NH and R³¹ is selected from

[chemical structure]

wherein p is an integer from 1-4, n is an integer from 0-3 and, R¹², R¹³, R¹⁴ and R¹⁵ are independently selected from hydrogen and optionally substituted C₁₋₆ alkyl; or
   R⁶ and R⁷ are linked to form a group

[chemical structure]

wherein R¹² and R¹³ are as hereinbefore defined and m is 1 or 2; and
   R⁹ and R¹⁰ are the same or different and each is selected from hydrogen or C₁₋₆ alkyl; and salts, solvates and physiologically functional derivatives thereof.

In some embodiments of the methods, the compound of Formula I is a compound wherein

R¹ is a straight chained C₁₋₆ alkyl group;
R² is a straight chained C₁₋₆ alkyl group;
R³ is hydrogen or a group OR¹¹ in which R¹¹ is hydrogen, optionally substituted C₁₋₆ alkyl or a C₁₋₆ alkylcarbonyl group;
R⁴ is optionally substituted phenyl;
R⁵, R⁶ and R⁸ are independently selected from hydrogen, C₁₋₄ alkyl optionally substituted by fluorine, C₁₋₄ alkoxy, halogen, or hydroxy;
R⁷ is selected from halogen, cyano, R¹⁵-acetylide, OR¹⁵, optionally substituted C₁₋₆ alkyl, COR¹⁵, CH(OH)R¹⁵, S(O)ₙR¹⁵, P(O)(OR¹⁵)₂, OCOR¹⁵, OCF₃, OCN, SCN, HNCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)ₚR¹⁵, O(CH₂)ₚSO₃R¹⁵, O(CH₂)ₚNR¹²R¹³ and O(CH₂)ₚN⁺R¹²R¹³R¹⁴;
wherein n, p and R¹² to R¹⁵ are as hereinbefore defined;
   with the proviso that at least two of R⁵ to R⁸ are not hydrogen; and
   salts solvates and physiologically functional derivatives thereof.

In some embodiments of the methods described herein, the compound of Formula I is a compound

wherein
R¹ is a straight chained C₁₋₆ alkyl group;
R² is a straight chained C₁₋₆ alkyl group;
R³ is hydrogen or a group OR¹¹ in which R¹¹ is hydrogen, optionally substituted C₁₋₆ alkyl or a C₁₋₆ alkylcarbonyl group;
R⁴ is un-substituted phenyl;
R⁵ is hydrogen or halogen;
R⁶ and R⁸ are independently selected from hydrogen, C₁₋₄ alkyl optionally substituted by fluorine, C₁₋₄ alkoxy, halogen, or hydroxy;
R⁷ is selected from OR¹⁵, S(O)ₙR¹⁵, OCOR¹⁵, OCF₃, OCN, SCN, CHO, OCH₂OR¹⁵, OCH═CHR¹⁵, O(CH₂CH₂O)nR¹⁵, O(CH₂)ₚSO₃R¹⁵, O(CH₂)ₚNR¹²R¹³ and O(CH₂)ₚN⁺R¹²R¹³R¹⁴ wherein p is an integer from 1-4, n is an integer from 0-3, and R¹², R¹³, R¹⁴, and R¹⁵ are independently selected from hydrogen and optionally substituted C₁₋₆ alkyl;
R⁹ and R¹⁰ are the same or different and each is selected from hydrogen or C₁₋₆ alkyl; and
salts, solvates and physiologically functional derivatives thereof.

In some embodiments of the methods, wherein the compound of Formula I is a compound

wherein
R¹ is methyl, ethyl or n-propyl;
R² is methyl, ethyl, n-propyl, n-butyl or n-pentyl;
R³ is hydrogen or a group OR¹¹ in which R¹¹ is hydrogen, optionally substituted C₁₋₆ alkyl or a C₁₋₆ alkylcarbonyl group;
R⁴ is un-substituted phenyl;
R⁵ is hydrogen;
R⁶ and R⁸ are independently selected from hydrogen, C₁₋₄ alkyl optionally substituted by fluorine, C₁₋₄ alkoxy, halogen, or hydroxy;
R⁷ is selected from OR¹⁵, S(O)ₙR¹⁵, OCOR¹⁵, OCF₃, OCN, SCN, CHO, OCH₂OR¹⁵, OCH═CHR¹⁵, O(CH₂CH₂O)nR¹⁵, O(CH₂)ₚSO₃R¹⁵, O(CH₂)ₚNR¹²R¹³ and O(CH₂)ₚN⁺R¹²R¹³R¹⁴ wherein p is an integer from 1-4, n is an integer from 0-3, and R¹², R¹³, R¹⁴, and R¹⁵ are independently selected from hydrogen and optionally substituted C₁₋₆ alkyl;
R⁹ and R¹⁰ are the same or different and each is selected from hydrogen or C₁₋₆ alkyl; and
salts, solvates and physiologically functional derivatives thereof.

In some embodiments of the methods, the compound of Formula I is a compound wherein

R¹ is methyl, ethyl or n-propyl;
R² is methyl, ethyl, n-propyl, n-butyl or n-pentyl;
R³ is hydrogen or a group OR¹¹ in which R¹¹ is hydrogen, optionally substituted C₁₋₆ alkyl or a C₁₋₆ alkylcarbonyl group;
R⁴ is un-substituted phenyl;
R⁵ is hydrogen;
R⁶ is C₁₋₄ alkoxy, halogen, or hydroxy;
R⁷ is OR¹⁵, wherein R¹⁵ is hydrogen or optionally substituted C₁₋₆ alkyl;
R⁸ is hydrogen or halogen;
R⁹ and R¹⁰ are the same or different and each is selected from hydrogen or C₁₋₆ alkyl; and salts, solvates and physiologically functional derivatives thereof.

In some embodiments of the methods, the compound of Formula I is
(3R,5R)-3-Butyl-3-ethyl-2,3,4,5-tetrahydro-7,8-dimethoxy-5-phenyl-1,4-benzothiazepine 1,1-dioxide;
(3R,5R)-3-Butyl-3-ethyl-2,3,4,5-tetrahydro-7,8-dimethoxy-5-phenyl-1,4-benzothiazepin-4-ol 1,1-dioxide;
(±)-Trans-3-butyl-3-ethyl-2,3,4,5-tetrahydro-7,8-dimethoxy-5-phenyl-1,4-benzothiazepine 1,1-dioxide;
(±)-Trans-3-butyl-3-ethyl-2,3,4,5-tetrahydro-7,8-dimethoxy-5-phenyl-1,4,-benzothiazepin-4-ol 1,1-dioxide;
(3R,5R)-7-Bromo-3-butyl-3-ethyl-2,3,4,5-tetrahydro-8-methoxy-5-phenyl-1,4-benzothiazepine 1,1-dioxide;
(3R,5R)-7-Bromo-3-butyl-3-ethyl-2,3,4,5-tetrahydro-8-methoxy-5-phenyl-1,4-benxothiaxepin-4-ol 1,1-dioxide;
(3R,5R)-3-Butyl-3-ethyl-2,3,4,5-tetrahydro-5-phenyl-1,4-benzothiazepine-7,8-diol 1,1-dioxide;
(3R,5R)-3-Butyl-3-ethyl-2,3,4,5-tetrahydro-8-methoxy-5-phenyl-1,4-benzothiazepin-7-ol 1,1-dioxide;
(3R,5R)-3-Butyl-3-ethyl-2,3,4,5-tetrahydro-7-methoxy-5-phenyl-1,4-benzothiazepin-8-ol 1,1-dioxide;
(±)-Trans-3-butyl-3-ethyl-2,3,4,5-tetrahydro-8-methoxy-5-phenyl-1,4-benzothiazepine 1,1-dioxide;
(±)-Trans-3-butyl-3-ethyl-2,3,4,5-tetrahydro-5-phenyl-1,4-benzothiazepin-8-ol 1,1-dioxide;
(±)-Trans-3-butyl-3-ethyl-2,3,4,5-tetrahydro-5-phenyl-1,4-benzothiazepine-4,8-diol;
(±)-Trans-3-butyl-3-ethyl-2,3,4,5-tetrahydro-5-phenyl-1,4-benzothiazepin-8-thiol 1,1-dioxide;
(±)-Trans-3-butyl-3-ethyl-2,3,4,5-tetrahydro-5-phenyl-1,4-benzothiazepin-8-sulfonic acid 1,1-dioxide;
(±)-Trans-3-butyl-3-ethyl-2,3,4,5-tetrahydro-8,9-dimethoxy-5-phenyl-1,4-benzothiazepine 1,1-dioxide;
(3R,5R)-3-butyl-7,8-diethoxy-2,3,4,5-tetrahydro-5-phenyl-1,4-benzothiazepine 1,1-dioxide;
(±)-Trans-3-butyl-8-ethoxy-3-ethyl-2,3,4,5-tetrahydro-5-phenyl-1,4-benzothiazepine 1,1-dioxide;
(±)-Trans-3-butyl-3-ethyl-2,3,4,5-tetrahydro-8-isopropoxy-5-phenyl-1,4-benzothiazepine 1,1-dioxide hydrochloride;
(±)-Trans-3-butyl-3-ethyl-2,3,4,5-tetrahydro-5-phenyl-1,4-benzothiazepin-8-carbaldehyde-1,1-dioxide;
3,3-Diethyl-2,3,4,5-tetrahydro-7,8-dimethoxy-5-phenyl-1,4-benzothiazepine 1,1-dioxide;
3,3-Diethyl-2,3,4,5-tetrahydro-8-methoxy-5-phenyl-1,4-benzothiazepine 1,1-dioxide;
3,3-Diethyl-2,3,4,5-tetrahydro-5-phenyl-1,4-benzothiazpin-4,8-diol 1,1-dioxide;
(RS)-3,3-Diethyl-2,3,4,5-tetrahydro-4-hydroxy-7,8-dimethoxy-5-phenyl-1,4-benzothiazepine 1,1-dioxide;
(±)-Trans-3-butyl-8-ethoxy-3-ethyl-2,3,4,5-tetrahydro-5-phenyl-1,4-benzothiazepin-4-ol-1-dioxide;
(±)-Trans-3-butyl-3-ethyl-2,3,4,5-tetrahydro-8-isopropoxy-5-phenyl-1,4-benzothiazepin-4-ol 1,1-dioxide;
(±)-Trans-3-butyl-3-ethyl-2,3,4,5-tetrahydro-7,8,9-trimethoxy-5-phenyl-1,4-benzothiazepin-4-ol 1,1-dioxide;
(3R,5R)-3-butyl-3-ethyl-2,3,4,5-tetrahydro-5-phenyl-1,4-benzothiazepin-4,7,8-triol 1,1-dioxide;
(±)-Trans-3-butyl-3-ethyl-2,3,4,5-tetrahydro-4,7,8-trimethoxy-5-phenyl-1,4-benzothiazepine 1,1-dioxide;
3,3-Diethyl-2,3,4,5-tetrahydro-5-phenyl-1,4-benzothiazepin-8-ol 1,1-dioxide;
3,3-Diethyl-2,3,4,5-tetrahydro-7-methoxy-5-phenyl-1,4-benzothiazepin-8-ol 1,1-dioxide;
3,3Dibutyl-2,3,4,5-tetrahydro-5-phenyl-1,4-benzothiazepin-8-ol 1,1-dioxide;
(±)-Trans-3-Butyl-3-ethyl-2,3,4,5-tetrahydro-1,1-dioxo-5-phenyl-1,4-benzothiazepin-8-yl hydrogen sulfate; or
3,3-Diethyl-2,3,4,5-tetrahydro-1,1-dioxo-5-phenyl-1,4-benzothiazepin-8-yl hydrogen sulfate.

In some embodiments, the compound of Formula I is

[chemical structure]

[chemical structure]

[chemical structure]

[chemical structure]

[chemical structure]

[chemical structure]

[chemical structure]

In some embodiments of the methods, the compound of Formula I is

[chemical structure]

In some embodiments, the compound of Formula I is not a structure shown as:

[chemical structure]

wherein m represents an integer of 1 or 2, and R³ and R⁴, which may be mutually different, each represents an alkyl group having 1 to 5 carbon atoms.

In some embodiments, an ASBTI suitable for the methods described herein is a compound of Formula II

[chemical structure]

wherein:
q is an integer from 1 to 4;
n is an integer from 0 to 2;
R¹ and R² are independently selected from the group consisting of H, alkyl, alkenyl, alkynyl, haloalkyl, alkylaryl, arylalkyl, alkoxy, alkoxyalkyl, dialkylamino, alkylthio, (polyalkyl)aryl, and cycloalkyl,
wherein alkyl, alkenyl, alkynyl, haloalkyl, alkylaryl, arylalkyl, alkoxy, alkoxyalkyl, dialkylamino, alkylthio, (polyalkyl)aryl, and cycloalkyl optionally are substituted with one or more substituents selected from the group consisting of OR⁹, NR⁹R¹⁰, N⁺R⁹R¹⁰R^{w}A⁻, SR⁹, S⁺R⁹R¹⁰A⁻, P⁺R⁹R¹⁰R¹¹A⁻, S(O)R⁹, SO₂R⁹, SO₃R⁹, CO₂R⁹, CN, halogen, oxo, and CONR⁹R¹⁰,
wherein alkyl, alkenyl, alkynyl, alkylaryl, alkoxy, alkoxyalkyl, (polyalkyl)aryl, and cycloalkyl optionally have one or more carbons replaced by O, NR⁹, N^{+F}R⁹R¹⁰A⁻, S, SO, SO₂, S⁺R⁹A⁻, P⁴R⁹R¹⁰A⁻, or phenylene,
wherein R⁹, R¹⁰, and R^{w} are independently selected from the group consisting of H, alkyl, alkenyl, alkynyl, cycloalkyl, aryl, acyl, heterocycle, ammoniumalkyl, arylalkyl, and alkylammoniumalkyl; or
R¹ and R² taken together with the carbon to which they are attached form C₃-C₁₀ cycloalkyl;
R³ and R⁴ are independently selected from the group consisting of H, alkyl, alkenyl, alkynyl, acyloxy, aryl, heterocycle, OR⁹, NR⁹R¹⁰, SR⁹, S(O)R⁹, SO₂R⁹, and SO₃R⁹, wherein R⁹ and R¹⁰ are as defined above; or
R³ and R⁴ together ═O, ═NOR¹¹, ═S, ═NNR¹¹R¹², ═NR⁹, or ═CR¹¹R¹²,
wherein R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ and R⁶ are independently selected from the group consisting of H, alkyl, alkenyl, alkynyl, aryl, cycloalkyl, heterocycle, quaternary heterocycle, quaternary heteroaryl, OR⁹, SR⁹, S(O)R⁹, SO₂R⁹, SO₃R⁹, and -L_{z}-K_{z};
wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, R¹⁵, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,

wherein:
A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, quaternary heteroarylalkyl, and -G-T-V—W, wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR, P⁺R⁹R¹⁰A⁻, P(O)R⁹, phenylene, carbohydrate, C₂-C₇ polyol, amino acid, peptide, or polypeptide, and
G, T and V are each independently a bond, —O—, —S—, —N(H)—, substituted or unsubstituted alkyl, —O-alkyl, —N(H)-alkyl, —C(O)N(H)—, —N(H)C(O)—, —N(H)C(O)N(H)—, substituted or unsubstituted alkenyl, substituted or unsubstituted alkynyl, substituted or unsubstituted aryl, substituted or unsubstituted arylalkyl, substituted or unsubstituted alkenylalkyl, alkynylalkyl, substituted or unsubstituted heteroalkyl, substituted or unsubstituted heterocycle, substituted or unsubstituted carboxyalkyl, substituted or unsubstituted carboalkoxyalkyl, or substituted or unsubstituted cycloalkyl, and
W is quaternary heterocycle, quaternary heteroaryl, quaternary heteroarylalkyl, N⁺R⁹R¹¹R¹²A⁻, P⁺R⁹R¹⁰R¹¹A⁻, OS(O)₂OM, or S⁺R⁹R¹⁰A⁻, and
R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O) R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and
one or more R^{x} are independently selected from the group consisting of H, alkyl, alkenyl, alkynyl, polyalkyl, acyloxy, aryl, arylalkyl, halogen, haloalkyl, cycloalkyl, heterocycle, heteroaryl, polyether, quaternary heterocycle, quaternary heteroaryl, OR¹³, NR¹³R¹⁴, SR¹³, S(O)R¹³, S(O)₂R¹³, SO₃R¹³, S⁴R¹³R¹⁴A⁻, NR¹³OR¹⁴, NR¹³NR¹⁴R¹⁵, NO₂, CO₂R¹³, CN, OM, SO₂OM, SO₂NR¹³R¹⁴, NR¹⁴C(O)R¹³, C(O)NR¹³R¹⁴, NR¹⁴C(O)R¹³, C(O)OM, COR¹³, OR¹⁸, S(O). NR¹⁸, NR¹³R¹⁸, NR¹⁸R¹⁴, N⁺R⁹R¹¹R¹²A⁻, P⁺R⁹R¹⁰, A⁻, amino acid, peptide, polypeptide, and carbohydrate,
wherein alkyl, alkenyl, alkynyl, cycloalkyl, aryl, polyalkyl, heterocycle, acyloxy, arylalkyl, haloalkyl, polyether, quaternary heterocycle, and quaternary heteroaryl can be further substituted with OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹¹R¹²A⁻, S⁺R⁹R¹⁰A⁻, or C(O)M, and
wherein R¹⁸ is selected from the group consisting of acyl, arylalkoxycarbonyl, arylalkyl, heterocycle, heteroaryl, alkyl,
wherein acyl, arylalkoxycarbonyl, arylalkyl, heterocycle, heteroaryl, alkyl, quaternary heterocycle, and quaternary heteroaryl optionally are substituted with one or more substituents selected from the group consisting of OR⁹, NR⁹R¹⁰, N⁺R⁹R¹¹R¹²A⁻, SR⁹, S(O)R⁹, SO₂R⁹, SO₃R⁹, oxo, CO₃R⁹, CN, halogen, CONR⁹R¹⁰, SO₃R⁹, SO₂OM, SO₂NR⁹R¹⁰, PO(OR¹⁶)OR¹⁷, and C(O)OM,
wherein in R^{x}, one or more carbons are optionally replaced by O, NR¹³, N⁺R¹³R¹⁴A⁻, S, SO, SO₂, S⁴R¹³A⁻, PR¹³, P(O)R¹³, P⁺R¹³R¹⁴A⁻, phenylene, amino acid, peptide, polypeptide, carbohydrate, polyether, or polyalkyl,
wherein in said polyalkyl, phenylene, amino acid, peptide, polypeptide, and carbohydrate, one or more carbons are optionally replaced by O, NR⁹, R⁹R¹⁰A⁻, S, SO, SO₂, S⁺R⁹A⁻, PR⁹, P⁺R⁹R¹⁰A⁻, or P(O)R⁹;
wherein quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
provided that both R⁵ and R⁶ cannot be hydrogen or SH;
provided that when R⁵ or R⁶ is phenyl, only one of R¹ or R² is H;
   provided that when q=1 and R^{x} is styryl, anilido, or anilinocarbonyl, only one of R⁵ or R⁶ is alkyl; or a
   pharmaceutically acceptable salt, solvate, or prodrug thereof.

In some embodiments of the methods, the compound of Formula II is a compound wherein
q is an integer from 1 to 4;
n is 2;
R¹ and R² are independently selected from the group consisting of H, alkyl, alkoxy, dialkylamino, and alkylthio,
wherein alkyl, alkoxy, dialkylamino, and alkylthio are optionally substituted with one or more substituents selected from the group consisting of OR⁹, NR⁹R¹⁰, SR⁹, SO₂R⁹, CO₂R⁹, CN, halogen, oxo, and CONR⁹R¹⁰;
each R⁹ and R¹⁰ are each independently selected from the group consisting of H, alkyl, cycloalkyl, aryl, acyl, heterocycle, and arylalkyl;
R³ and R⁴ are independently selected from the group consisting of H, alkyl, acyloxy, OR⁹, NR⁹R¹⁰, SR⁹, and SO₂R⁹, wherein R⁹ and R¹⁰ are as defined above;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ and R⁶ are independently selected from the group consisting of H, alkyl, aryl, cycloalkyl, heterocycle, and -L_{z}-K_{z};
wherein z is 1 or 2; each L is independently a substituted or unsubstituted alkyl, a substituted or unsubstituted heteroalkyl, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, aryl, cycloalkyl, and heterocycle can be substituted with one or more substituent groups independently selected from the group consisting of alkyl, aryl, haloalkyl, cycloalkyl, heterocycle, arylalkyl, quaternary heterocycle, quaternary heteroaryl, halogen, oxo, OR¹³, OR¹³R¹⁴, NR¹³R¹⁴, SR¹³, SO₂R¹³, NR¹³NR¹⁴R¹⁵, NO₂, CO₂R¹³, CN, OM, and CR¹³,
wherein:
A⁻ is a pharmaceutically acceptable anion and M is a pharmaceutically acceptable cation;
R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl, wherein R¹³, R¹⁴ and R¹⁵ are optionally substituted with one or more groups selected from the group consisting of quaternary heterocycle, quaternary heteroaryl, OR⁹, NR⁹R¹⁰, N⁺R⁹R¹¹R¹² A⁻, SR⁹, S(O)R⁹, SO₂R⁹, SO₃R⁹, oxo, CO₂R⁹, CN, halogen, and CONR⁹R¹⁰; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and one or more R^{x} are independently selected from the group consisting of H, alkyl, acyloxy, aryl, arylalkyl, halogen, haloalkyl, cycloalkyl, heterocycle, heteroaryl, OR¹³, NR¹³R¹⁴, SR¹³, S(O)₂R¹³, NR¹³NR¹⁴R¹⁵, NO₂, CO₂R¹³, CN, SO₂NR¹³R¹⁴, NR¹⁴C(O)R¹³, C(O)NR¹³R¹⁴, NR¹⁴C(O)R¹³, and COR¹³;
   provided that both R⁵ and R⁶ cannot be hydrogen;
   provided that when R⁵ or R⁶ is phenyl, only one of R¹ or R² is H;
   provided that when q=1 and R^{x} is styryl, anilido, or anilinocarbonyl, only one of R⁵ or R⁶ is alkyl; or a pharmaceutically acceptable salt, solvate, or prodrug thereof.

In some embodiments, the compound of Formula II is a compound wherein
q is 1;
n is 2;
R^{x} is N(CH₃)₂;
R⁷ and R⁸ are independently H;
R¹ and R² is alkyl;
R³ is H, and R⁴ is OH;
R⁵ is H, and R⁶ is selected from the group consisting of alkyl, alkenyl, alkynyl, aryl, cycloalkyl, heterocycle, quaternary heterocycle, quaternary heteroaryl, OR⁹, SR⁹, S(O)R⁹, SO₂R⁹, SO₃R⁹, and -L_{z}-K_{z};
wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, R¹⁵, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
wherein A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, quaternary heteroarylalkyl, and -G-T-V—W,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR, P⁺R⁹R¹⁰A⁻, P(O)R⁹, phenylene, carbohydrate, C₂-C₇ polyol, amino acid, peptide, or polypeptide, and
G, T and V are each independently a bond, —O—, —S—, —N(H)—, substituted or unsubstituted alkyl, —O-alkyl, —N(H)-alkyl, —C(O)N(H)—, —N(H)C(O)—, —N(H)C(O)N(H)—, substituted or unsubstituted alkenyl, substituted or unsubstituted alkynyl, substituted or unsubstituted aryl, substituted or unsubstituted arylalkyl, substituted or unsubstituted alkenylalkyl, alkynylalkyl, substituted or unsubstituted heteroalkyl, substituted or unsubstituted heterocycle, substituted or unsubstituted carboxyalkyl, substituted or unsubstituted carboalkoxyalkyl, or substituted or unsubstituted cycloalkyl, and
W i s quaternary heterocycle, quaternary heteroaryl, quaternary heteroarylalkyl, N⁺R⁹R¹¹R¹²A⁻, P⁺R⁹R¹⁰R¹¹A⁻, OS(O)₂OM, or S⁺R⁹R¹⁰A⁻, and
R⁹ and R¹⁰ are independently selected from the group consisting of H, alkyl, alkenyl, alkynyl, cycloalkyl, aryl, acyl, heterocycle, ammoniumalkyl, arylalkyl, and alkylammoniumalkyl;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O) R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl;
or a pharmaceutically acceptable salt, solvate, or prodrug thereof.

In some embodiments, the compound of Formula II is a compound wherein
q is 1;
n is 2;
R^{x} is N(CH₃)₂;
R⁷ and R⁸ are independently H;
R¹ and R² is independently C₁-C₄ alkyl;
R³ is H, and R⁴ is OH;
R⁵ is H, and R⁶ is arylsubstituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, R¹⁵, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
wherein A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, quaternary heteroarylalkyl, and -G-T-V—W,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR, P⁺R⁹R¹⁰A⁻, P(O)R⁹, phenylene, carbohydrate, C₂-C₇ polyol, amino acid, peptide, or polypeptide, and
G, T and V are each independently a bond, —O—, —S—, —N(H)—, substituted or unsubstituted alkyl, —O-alkyl, —N(H)-alkyl, —C(O)N(H)—, —N(H)C(O)—, —N(H)C(O)N(H)—, substituted or unsubstituted alkenyl, substituted or unsubstituted alkynyl, substituted or unsubstituted aryl, substituted or unsubstituted arylalkyl, substituted or unsubstituted alkenylalkyl, alkynylalkyl, substituted or unsubstituted heteroalkyl, substituted or unsubstituted heterocycle, substituted or unsubstituted carboxyalkyl, substituted or unsubstituted carboalkoxyalkyl, or substituted or unsubstituted cycloalkyl, and
W i s quaternary heterocycle, quaternary heteroaryl, quaternary heteroarylalkyl, N⁺R⁹R¹¹R¹²A⁻, P⁺R⁹R¹⁰R¹¹A⁻, OS(O)₂OM, or S⁺R⁹R¹⁰A⁻, and
R⁹ and R¹⁰ are independently selected from the group consisting of H, alkyl, alkenyl, alkynyl, cycloalkyl, aryl, acyl, heterocycle, ammoniumalkyl, arylalkyl, and alkylammoniumalkyl;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O) R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl;
or a pharmaceutically acceptable salt, solvate, or prodrug thereof.

In some embodiments of the methods, the compound of Formula II is a compound wherein

R⁵ and R⁶ are independently selected from the group consisting of H, aryl, heterocycle, quaternary heterocycle, and quaternary heteroaryl
wherein the aryl, heteroaryl, quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, N⁺R⁹R¹¹R¹²A⁻ and -L_{z}-K_{z}.

In some embodiments of the methods, the compound of Formula II is a compound wherein

### R⁵ or R⁶ is —Ar—(R^{y}),

t is an integer from 0 to 5;
Ar is selected from the group consisting of phenyl, thiophenyl, pyridyl, piperazinyl, piperonyl, pyrrolyl, naphthyl, furanyl, anthracenyl, quinolinyl, isoquinolinyl, quinoxalinyl, imidazolyl, pyrazolyl, oxazolyl, isoxazolyl, pyrimidinyl, thiazolyl, triazolyl, isothiazolyl, indolyl, benzoimidazolyl, benzoxazolyl, benzothiazolyl, and benzoisothiazolyl; and
one or more R^{y} are independently selected from the group consisting of alkyl, alkenyl, alkynyl, polyalkyl, polyether, aryl, halo alkyl, cycloalkyl, heterocycle, arylalkyl, halogen, oxo, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, P(O)R¹³R¹⁴, P⁺R¹³R¹⁴R¹⁵A⁻. P(OR¹³)OR¹⁴, S⁺R¹³R¹⁴A⁻, N⁺R⁹R¹¹R¹²A⁻ and -L_{z}-K_{z};
wherein said alkyl, alkenyl, alkynyl, polyalkyl, polyether, aryl, haloalkyl, cycloalkyl, and heterocycle can be further substituted with one or more substituent groups selected from the group consisting of OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸A⁻, and P(O)(OR⁷)OR⁸, and or phenylene;
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene.

In some embodiments of the methods, the compound of Formula II is a compound wherein

### R⁵ or R⁶ is

[chemical structure]

In some embodiments of the methods, the compound of Formula II is a compound wherein n is 1 or 2. In some embodiments of the methods, the compound of Formula II is a compound wherein R¹ and R² are independently H or C₁₋₇ alkyl. In some embodiments of the methods, the compound of Formula II is a compound wherein each C₁₋₇ alkyl is independently ethyl, n-propyl, n-butyl, or isobutyl. In some embodiments of the methods, the compound of Formula II is a compound wherein R³ and R⁴ are independently H or OR⁹. In some embodiments of the methods, compound of Formula II is a compound wherein R⁹ is H

In some embodiments of the methods, the compound of Formula II is a compound wherein one or more R^{x} are in the 7-, 8- or 9-position of the benzo ring of Formula II. In some embodiments of the methods, the compound of Formula II is a compound wherein R^{x} is in the 7-position of the benzo ring of Formula II. In some embodiments of the methods, the compound of Formula II is a compound wherein one or more R^{x} are independently selected from OR¹³ and NR¹³R¹⁴.

In some embodiments of the methods, the compound of Formula II is a compound wherein:
q is 1 or 2;
n is 2;
R¹ and R² are each alkyl;
R³ is hydroxy;
R⁴ and R⁶ are hydrogen;
R⁵ has the formula

[chemical structure]

wherein
t is an integer from 0 to 5;
   one or more R^{Y} are OR¹³ or OR¹³R¹⁴;
   R¹³ and R¹⁴ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, and quaternary heteroarylalkyl;
   wherein said alkyl, alkenyl, alkynyl, arylalkyl, heterocycle, and polyalkyl groups optionally have one or more carbons replaced by O, NR⁹, N⁺R⁹R¹⁰A⁻, S, SO, SO₂, S⁺R⁹A⁻, PR⁹, P⁺R⁹R¹⁰A⁻, P(O)R⁹, phenylene, carbohydrate, amino acid, peptide, or polypeptide;
R¹³ and R¹⁴ are optionally substituted with one or more groups independently selected from the group consisting of sulfoalkyl, quaternary heterocycle, quaternary heteroaryl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein A is a pharmaceutically acceptable anion, and M is a pharmaceutically acceptable cation,
R⁹ and R¹⁰ are independently selected from the group consisting of H, alkyl, alkenyl, alkynyl, cycloalkyl, aryl, acyl, heterocycle, ammoniumalkyl, arylalkyl, and alkylammoniumalkyl;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH; or
R¹¹ and R¹² together with the nitrogen or carbon atom to which they are attached form a cyclic ring; and
R¹⁶ and R¹⁷ are independently selected from the substituents constituting R⁹ and M;
R⁷ and R⁸ are hydrogen; and
one or more R^{x} are independently selected from the group consisting of alkoxy, alkylamino and dialkylamino and W—R³¹, wherein W is O or NH and R³¹ is selected from

[chemical structure]

or a pharmaceutically acceptable salt, solvate, or prodrug thereof.

In some embodiments, a compound of Formula II is

[chemical structure]

[chemical structure]

or the like.

In some embodiments, a compound of Formula II is

[chemical structure]

In some embodiments of the methods, the compound of Formula II is

[chemical structure]

In certain embodiments, ASBTIs suitable for the methods described herein are non-systemic analogs of Compound 100C. Certain compounds provided herein are Compound 100C analogues modified or substituted to comprise a charged group. In specific embodiments, the Compound 100C analogues are modified or substituted with a charged group that is an ammonium group (e.g., a cyclic ar acyclic ammonium group). In certain embodiments, the ammonium group is a non-protic ammonium group that contains a quaternary nitrogen.

In some embodiments, a compound of Formula II is

[chemical structure]

In some embodiments, a compound of Formula II is 1-[[5-[[3-[(3S,4R,5R)-3-butyl-7-(dimethylamino)-3-ethyl-2,3,4,5-tetrahydro-4-hydroxy-1,1-dioxido-1-benzothiepin-5yl]phenyl]amino]-5-oxopentyl]amino]-1-deoxy-D-glucitol or SA HMR1741 (a.k.a. BARI-1741).

In some embodiments, a compound of Formula II is

[chemical structure]

In some embodiments, a compound of Formula II is potassium((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl)sulphate ethanolate, hydrate or SAR548304B (a.k.a. SAR-548304).

In some embodiments, an ASBTI suitable for the methods described herein is a compound of Formula III:

[chemical structure]

wherein:
each R¹, R² is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R¹ and R² together with the nitrogen to which they are attached form a 3-8-membered ring that is optionally substituted with R⁸;
each R³, R⁴ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
R⁵ is H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl,
each R⁶, R⁷ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R⁶ and R⁷ taken together form a bond;
each X is independently NH, S, or O;
each Y is independently NH, S, or O;
R⁸ is substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-7;
K is a moiety that prevents systemic absorption;
provided that at least one of R¹, R², R³ or R⁴ is -L-K;

or a pharmaceutically acceptable prodrug thereof.

In some embodiments of a compound of Formula III, R¹ and R³ are -L-K. In some embodiments, R¹, R² and R³ are -L-K.

In some embodiments, at least one of R¹, R², R³, R⁴, R⁵, R⁶ and R⁷ is H. In certain embodiments, R⁵, R⁶, R⁷ are H and R¹, R², R³ and R⁴ are alkyl, aryl, alkyl-aryl, or heteroalkyl. In some embodiments, R¹ and R² are H. In some embodiments, R¹, R², R⁵, R⁶ and R⁷ are H. In some embodiments, R⁶ and R⁷ together form a bond. In certain embodiments, R⁵, R⁶ and R⁷ are H, alkyl or O-alkyl.

In some embodiments, R¹ and R³ are -L-K. In some embodiments, R¹, R² and R³ are -L-K. In some embodiments, R³ and R⁴ are -L-K. In some embodiments, R¹ and R² together with the nitrogen to which they are attached form a 3-8 membered ring and the ring is substituted with -L-K. In some embodiments, R¹ or R² or R³ or R⁴ are aryl optionally substituted with -L-K. In some embodiments, R¹ or R² or R³ or R⁴ are alkyl optionally substituted with -L-K. In some embodiments, R¹ or R² or R³ or R⁴ are alky-aryl optionally substituted with -L-K. In some embodiments, R¹ or R² or R³ or R⁴ are heteroalkyl optionally substituted with -L-K.

In some embodiments, L is a C₁-C₇alkyl. In some embodiments, L is heteroalkyl. In certain embodiments, L is C₁-C₇alkyl-aryl. In some embodiments, L is C₁-C₇alkyl-aryl-C₁-C₇alkyl.

In certain embodiments, K is a non-protic charged group. In some specific embodiments, each K is a ammonium group. In some embodiments, each K is a cyclic non-protic ammonium group. In some embodiments, each K is an acyclic non-protic ammonium group.

In certain embodiments, each K is a cyclic non-protic ammonium group of structure:

[chemical structure]

In certain embodiments, K is an acyclic non-protic ammonium group of structure:

[chemical structure]

wherein p, q, R⁹, R¹⁰ and Z are as defined above. In certain embodiments, p is 1. In other embodiments, p is 2. In further embodiments, p is 3. In some embodiments, q is 0. In other embodiments, q is 1. In some other embodiments, q is 2.

The compounds further comprise 1, 2, 3 or 4 anionic counterions selected from Cl⁻, Br⁻, I⁻, R¹¹SO₃⁻, (SO₃⁻—R¹¹—SO₃⁻), R¹¹CO₂, (CO₂⁻—R¹¹—CO₂⁻), (R¹¹)₂(P═O)O⁻ and (R¹¹)(P═O)O₂²⁻ wherein R¹¹ is as defined above. In some embodiments, the counterion is Cl⁻, Br⁻, I⁻, CH₂CO₂⁻, CH₃SO₃⁻, or C₆H₅SO₃⁻ or CO₂⁻—(CH₂)₂—CO₂⁻. In some embodiments, the compound of Formula III has one K group and one counterion. In other embodiments, the compound of Formula III has one K group, and two molecules of the compound of Formula III have one counterion. In yet other embodiments, the compound of Formula III has two K groups and two counterions. In some other embodiments, the compound of Formula III has one K group comprising two ammonium groups and two counterions.

Also described herein are compounds having the Formula IIIA:

[chemical structure]

wherein:
each R¹, R² is independently H, substituted or unsubstituted alkyl, or -L-K; or R¹ and R² together with the nitrogen to which they are attached form a 3-8-membered ring that is optionally substituted with R⁸;
and R³, R⁴, R⁸, L and K are as defined above.

In some embodiments of compounds of Formula IIIA, L is Aₙ, wherein each A is substituted or unsubstituted alkyl, or substituted or unsubstituted heteroalkyl, and n is 0-7. In certain specific embodiments of the compound of Formula IIIA, R¹ is H. In some embodiments of Formula IIIA, R¹ and R² together with the nitrogen to which they are attached form a 3-8-membered ring that is optionally substituted with -L-K.

Also described herein are compounds having the Formula IIIB:

[chemical structure]

wherein:
each R³, R⁴ is independently H, substituted or unsubstituted alkyl, substituted or unsubstituted heteroalkyl, substituted or unsubstituted aryl, substituted or unsubstituted alkyl-aryl, or -L-K;
and R¹, R², L and K are as defined above.

In certain embodiments of Formula MB, R³ is H. In certain embodiments, R³ and R⁴ are each -L-K. In some embodiments, R³ is H and R⁴ is substituted or unsubstituted alkyl, substituted or unsubstituted heteroalkyl, substituted or unsubstituted aryl, substituted or unsubstituted alkyl-aryl containing one or two -L-K groups.

In some embodiments, an ASBTI suitable for the methods described herein is a compound of Formula IIIC

[chemical structure]

wherein:
each R¹, R² is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R¹ and R² together with the nitrogen to which they are attached form a 3-8-membered ring that is optionally substituted with R⁸;
each R³, R⁴ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
R⁵ is H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl,
each R⁶, R⁷ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R⁶ and R⁷ taken together form a bond;
each X is independently NH, S, or O;
each Y is independently NH, S, or O;
R⁸ is substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
L is Aₙ, wherein
   each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
   n is 0-7;
K is a moiety that prevents systemic absorption;
   or a pharmaceutically acceptable salt thereof.

In some specific embodiments of Formula I, II or III, K is selected from

[chemical structure]

In some embodiments, an ASBTI suitable for the methods described herein is a compound of Formula IV:

[chemical structure]

wherein

R¹ is a straight chain C₁₋₆ alkyl group;

R² is a straight chain C₁₋₆ alkyl group;

R³ is hydrogen or a group OR¹¹ in which R¹¹ is hydrogen, optionally substituted C₁₋₆ alkyl or a C₁₋₆ alkylcarbonyl group;

R⁴ is pyridyl or an optionally substituted phenyl;

R⁵, R⁶ and R⁸ are the same or different and each is selected from:
hydrogen, halogen, cyano, R¹⁵-acetylide, OR¹⁵, optionally substituted C₁₋₆ alkyl, COR¹⁵, CH(OH)R¹⁵, S(O)R¹⁵, P(O)(OR¹⁵)₂, OCOR¹⁵, OCF₃, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)R¹⁵, O(CH₂)ₚSO₃R¹⁵, O(CH₂)ₚNR¹²R¹³ and O(CH₂)ₚN⁺R¹²R¹³R¹⁴ wherein

p is an integer from 1-4,

n is an integer from 0-3 and

R¹², R¹³, R¹⁴ and R¹⁵ are independently selected from hydrogen and optionally substituted C″ alkyl;

R⁷ is a group of the formula

[chemical structure]

wherein the hydroxyl groups may be substituted by acetyl, benzyl, or —(C₁-C₆)-alkyl-R¹⁷,
wherein the alkyl group may be substituted with one or more hydroxyl groups;

R¹⁶ is —COOH, —CH₂—OH, —CH₂—O-Acetyl, —COOMe or —COOEt;

R¹⁷ is H, —OH, —NH₂, —COOH or COOR¹⁸;

R¹⁸ is (C₁-C₄)-alkyl or —NH—(C₁-C₄)-alkyl;

X is —NH or —O—; and

R⁹ and R¹⁰ are the same or different and each is hydrogen or C₁-C₆ alkyl; and salts thereof.

In some embodiments, a compound of Formula IV has the structure of Formula IVA or Formula IVB:

[chemical structure]

In some embodiments, a compound of Formula IV has the structure of Formula IVC:

[chemical structure]

In some embodiments of Formula IV, X is O and R⁷ is selected from

[chemical structure]

In some embodiments, a compound of Formula IV is:

[chemical structure]

In some embodiments, an ASBTI suitable for the methods described herein is a compound of Formula V:

[chemical structure]

wherein:

R^{v} is selected from hydrogen or C₁₋₆alkyl;

One of R¹ and R² are selected from hydrogen or C₁₋₆alkyl and the other is selected from C₁₋₆alkyl;

R^{x} and R^{y} are independently selected from hydrogen, hydroxy, amino, mercapto, C₁₋₆alkyl, C₁₋₆ alkoxy, N—(C₁₋₆alkyl)amino, N,N—(C₁₋₆alkyl)₂amino, C₁₋₆alkylS(O)ₐ wherein a is 0 to 2;

R^{z} is selected from halo, nitro, cyano, hydroxy, amino, carboxy, carbamoyl, mercapto, sulphamoyl, C₁₋₆alkyl, C₂₋₆alkenyl, C₂₋₆ alkynyl, C₁₋₆ alkoxy, C₁₋₆ alkanoyl, C₁₋₆alkanoyloxy, N—(C₁₋₆ alkyl)amino, N,N—(C₁₋₆alkyl)₂amino, C₁₋₆alkanoylamino, N—(C₁₋₆alkyl)carbamoyl, N,N—(C₁₋₆ alkyl)₂carbamoyl, C₁₋₆alkylS(O)ₐ wherein a is 0 to 2, C₁₋₆alkoxycarbonyl, N—(C₁₋₆-alkyl)sulphamoyl and N,N—(C₁₋₆alkyl)₂sulphamoyl;

n is 0-5;

one of R⁴ and R⁵ is a group of formula (VA):

[chemical structure]

R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆ alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl;
wherein R³ and R⁶ and the other of R⁴ and R⁵ may be optionally substituted on carbon by one or more R¹⁷;

X is —O—, —N(R^{a})—, —S(O)_{b}— or —CH(R^{a})—;
wherein R^{a} is hydrogen or C₁₋₆alkyl and b is 0-2;

Ring A is aryl or heteroaryl;
wherein Ring A is optionally substituted on carbon by one or more substituents selected from R¹⁸;

R⁷ is hydrogen, C₁₋₆alkyl, carbocyclyl or heterocyclyl;
wherein R⁷ is optionally substituted on carbon by one or more substituents selected from R¹⁹; and wherein if said heterocyclyl contains an —NH— group, that nitrogen may be optionally substituted by a group selected from R²⁰;

R⁸ is hydrogen or C₁₋₆-alkyl;

R⁹ is hydrogen or C₁₋₆alkyl;

R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkynyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹(C₁₋₁₀alkylene)_{q}- or heterocyclyl-(C₁₋₁₀alkylene)ᵣ-R²²(C₁₋₁₀alkylene)ₛ-; wherein R¹⁹ is optionally substituted on carbon by one or more substituents selected from R²³; and wherein if said heterocyclyl contains an NH group, that nitrogen may be optionally substituted by a group selected from R²⁴; or R¹⁹ is a group of formula (VB):

[chemical structure]

wherein:

R¹¹ is hydrogen or C₁₋₆-alkyl;

R¹² and R¹³ are independently selected from hydrogen, halo, carbamoyl, sulphamoyl, C₁₋₁₀alkyl, C₂₋₁₀alkynyl, C₂₋₁₀alkynyl, C₁₋₁₀alkanoyl, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;

R¹⁴ is selected from hydrogen, halo, carbamoyl, sulphamoyl, hydroxyaminocarbonyl, C₁₋₁₀alkyl, C₂₋₁₀alkenyl, C₂₋₁₀alkynyl, C₁₋₁₀alkanoyl, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷(C₁₋₁₀alkylene)_{q}- or heterocyclyl-(C₁₋₁₀alkylene)ᵣ-R⁸²—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VC):

[chemical structure]

R¹⁵ is hydrogen or C₁₋₆alkyl; and R¹⁶ is hydrogen or C₁₋₆alkyl; wherein R¹⁶ may be optionally substituted on carbon by one or more groups selected from R³¹;

or R¹⁵ and R¹⁶ together with the nitrogen to which they are attached form a heterocyclyl; wherein said heterocyclyl may be optionally substituted on carbon by one or more R³⁷; and wherein if said heterocyclyl contains an —NH— group, that nitrogen may be optionally substituted by a group selected from R³⁸;

m is 1-3; wherein the values of R⁷ may be the same or different;

R¹⁷, R¹⁸, R¹⁹, R²³, R²⁵, R²⁹, R³¹ and R³⁷ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R¹⁷, R¹⁸, R¹⁹, R²³, R²⁵, R²⁹, R³¹ and R³⁷ may be independently optionally substituted on carbon by one or more R³⁴; and wherein if said heterocyclyl contains an —NH— group, that nitrogen may be optionally substituted by a group selected from R³⁵;

R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;

p, q, r and s are independently selected from 0-2;

R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;

R²⁰, R²⁴, R²⁶, R³⁰, R³⁵ and R³⁸ are independently selected from C₁₋₆alkyl, C₁₋₆alkanoyl, C₁₋₆ alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; and

wherein a “heteroaryl” is a totally unsaturated, mono or bicyclic ring containing 3-12 atoms of which at least one atom is chosen from nitrogen, sulphur and oxygen, which heteroaryl may, unless otherwise specified, be carbon or nitrogen linked;

wherein a “heterocyclyl” is a saturated, partially saturated or unsaturated, mono or bicyclic ring containing 3-12 atoms of which at least one atom is chosen from nitrogen, sulphur and oxygen, which heterocyclyl may, unless otherwise specified, be carbon or nitrogen linked, wherein a —CH₂— group can optionally be replaced by a —C(O)— group, and a ring sulphur atom may be optionally oxidized to form an S-oxide; and

wherein a “carbocyclyl” is a saturated, partially saturated or unsaturated, mono or bicyclic carbon ring that contains 3-12 atoms; wherein a —CH₂— group can optionally be replaced by a —C(O) group;

or a pharmaceutically acceptable salt or in vivo hydrolysable ester or amide formed on an available carboxy or hydroxy group thereof.

In some embodiments, R⁴ and R⁵ is not S—CH₃ and/or

[chemical structure]

wherein R¹ is H or hydroxyl; and R² is H, CH₃, —CH₂CH₃, —CH₂CH₂CH₃, —CH₂CH₂CH₂CH₃, —CH(CH₃)₂, —CH₂CH(CH₃)₂, —CH(CH₃)CH₂CH₃, —CH₂OH, —CH₂OCH₃, —CH(OH)CH₃, —CH₂SCH₃, or —CH₂CH₂SCH₃.

In some embodiments, compound of Formula V is not 1,1-dioxo-3,3-dibutyl-5-phenyl-7-methylthio-8-(N-{(R)-α-[N—((R)-1-carboxy-2-methylthio-ethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxybutyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxyethyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((R)-1-carboxy-2-methylthioethyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxy-2-methylpropyl)carbamoyl]benzyl}carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]-4-hydroxybenzyl}carbamoylmethoxy)-2,3,4,5-tetrahydro-1,2,5-benzothiadiazepine; 1,1-dioxo-3,3-dibutyl-5-phenyl-7-methylthio-8-[N-{(R)-α-carboxy4-hydroxybenzyl}carbamoylmethoxy]-2,3,4,5-tetrahydro-1,2,5-benzothiadiazepine; or 1,1-dioxo-3,3-dibutyl-5-phenyl-7-methylthio-8-(N-{(R)-α-[N-(carboxymethyl)carbamoyl]benzyl}carbamoylmethoxy)-2,3,4,5-tetrahydro-1,2,5-benzothiadiazepine

In some embodiments, compound of Formula V is not

[chemical structure]

In some embodiments, an ASBTI suitable for the methods described herein is a compound of Formula VI:

[chemical structure]

wherein:

R^{v} and R^{w} are independently selected from hydrogen or C₁₋₆alkyl;

one of R¹ and R² is selected from hydrogen or C₁₋₆alkyl and the other is selected from C₁₋₆alkyl;

R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;

R^{z} is selected from halo, nitro, cyano, hydroxy, amino, carboxy, carbamoyl, mercapto, sulphamoyl, C₁₋₆alkyl, C₂₋₆alkenyl, C₂₋₆ alkynyl, C₁₋₆ alkoxy, C₁₋₆ alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆ alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl;

n is 0-5;

one of R⁴ and R⁵ is a group of formula (VIA):

[chemical structure]

R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆ alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl and N,N—(C₁₋₆alkyl)₂sulphamoyl; wherein R³ and R⁶ and the other of R⁴ and R⁵ may be optionally substituted on carbon by one or more R¹⁷;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted on carbon by one or more substituents selected from R¹⁸;

R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;

R⁸ is hydrogen or C₁₋₆alkyl;

R⁹ is hydrogen or C₁₋₆alkyl;

R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹(C₁₋₁₀alkylene)_{q}- or heterocyclyl-(C₁₋₁₀alkylene)ᵣ-R²²(C₁₋₁₀alkylene)ₛ-; wherein R¹⁹ is optionally substituted on carbon by one or more substituents selected from R²³; and wherein if said heterocyclyl contains an NH group, that nitrogen may be optionally substituted by a group selected from R²⁴; or R¹⁹ is a group of formula (VIB):

[chemical structure]

wherein:

R¹¹ is hydrogen or C₁₋₆alkyl;

R¹² and R¹³ are independently selected from hydrogen, halo, nitro, cyano, hydroxy, amino, carbamoyl, mercapto, sulphamoyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an NH group, that nitrogen may be optionally substituted by a group selected from R²⁶;

R¹⁴ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VIC):

[chemical structure]

R¹⁵ is hydrogen or C₁₋₆alkyl;

R¹⁶ is hydrogen or C₁₋₆alkyl; wherein R¹⁶ may be optionally substituted on carbon by one or more groups selected from R³¹;

n is 1-3; wherein the values of R⁷ may be the same or different;

R¹⁷, R¹⁸, R¹⁹, R²³, R²⁵, R²⁹ or R³¹ are independently selected from halo, nitro, cyano, hydroxy, amino, carbamoyl, mercapto, sulphamoyl, hydroxyaminocarbonyl, amidino, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, (C₁₋₁₀alkyl)₃silyl, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R¹⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;

R²¹, R²², R²⁷, R²⁸, R³² or R³³ are independently selected from —O—, —NR³⁶—, —S(O)—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;

p, q, r and s are independently selected from 0-2;

R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;

R²⁰, R²⁴, R²⁶, R³⁰ or R³⁵ are independently selected from C₁₋₆alkyl, C₁₋₆alkanoyl, C₁₋₆ alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

or a pharmaceutically acceptable salt, solvate or solvate of such a salt, or an in vivo hydrolysable ester formed on an available carboxy or hydroxy thereof, or an in vivo hydrolysable amide formed on an available carboxy thereof.

In some embodiments, a compound of Formula VI has the structure of Formula VID:

[chemical structure]

wherein:

R¹ and R² are independently selected from C₁₋₆alkyl; one of R⁴ and R⁵ is a group of formula (VIE):

[chemical structure]

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl and N,N—(C₁₋₄alkyl)₂sulphamoyl; wherein R³ and R⁶ and the other of R⁴ and R⁵ may be optionally substituted on carbon by one or more R¹⁴;

R⁷ is carboxy, sulpho, sulphino, phosphono, —P(O)(OR^{a})(OR^{b}), P(O)(OH)(ORₐ), P(O)(OH)(R^{a}) or P(O)(OR^{a})(R^{b}), wherein R^{a} and R^{b} are independently selected from C₁₋₆alkyl; or R⁷ is a group of formula (VIF):

[chemical structure]

R⁸ and R⁹ are independently hydrogen, C₁₋₄alkyl or a saturated cyclic group, or R⁸ and R⁹ together form C₂₋₆alkylene; wherein R⁸ and R⁹ or R⁸ and R⁹ together may be independently optionally substituted on carbon by one or more substituents selected from R¹⁵; and wherein if said saturated cyclic group contains an NH moiety, that nitrogen may be optionally substituted by one or more R²⁰;

R¹⁹ is hydrogen or C₁₋₄alkyl; wherein R¹⁹ is optionally substituted on carbon by one or more substituents selected from R²⁴;

R¹¹ is hydrogen, C¹⁻⁴alkyl, carbocyclyl or heterocyclyl; wherein R¹¹ is optionally substituted on carbon by one or more substituents selected from R¹⁶; and wherein if said heterocyclyl contains an NH moiety, that nitrogen may be optionally substituted by one or more R²¹;

R¹² is hydrogen or C₁₋₄alkyl, carbocyclyl or heterocyclyl; wherein R¹² optionally substituted on carbon by one or more substituents selected from R¹⁷; and wherein if said heterocyclyl contains an NH moiety, that nitrogen may be optionally substituted by one or more R²²;

R¹³ is carboxy, sulpho, sulphino, phosphono, —P(O)(OR^{c})(OR^{d}), —P(O)(OH)(OR^{c}), —P(O)(OH)(R^{c}) or —P(O)(OR^{c})(R^{d}) wherein R^{c} and R^{d} are independently selected from C₁₋₆alkyl;

m is 1-3; wherein the values of R⁸ and R⁹ may be the same or different;

n is 1-3; wherein the values of R¹¹ may be the same or different;

p is 1-3; wherein the values of R¹² may be the same or different;

R¹⁴ and R¹⁶ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄ alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄ alkyl)sulphamoyl and N,N—(C₁₋₄alkyl)₂sulphamoyl; wherein R¹⁴ and R¹⁶ may be independently optionally substituted on carbon by one or more R¹⁸;

R¹⁵ and R¹⁷ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄ alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄ alkyl)sulphamoyl and N,N—(C₁₋₄alkyl)₂sulphamoyl, carbocyclyl, heterocyclyl, sulpho, sulphino, amidino, phosphono, —P(O)(OR^{e})(OR^{f}), —P(O)(OH)(OR^{e}), —P(O)(OH)(R^{e}) or —P(O)(OR^{e})(R^{f}) wherein Re and R^{f} are independently selected from C₁₋₆alkyl; wherein R¹⁵ and R¹⁷ may be independently optionally substituted on carbon by one or more R¹⁹; and wherein if said heterocyclyl contains an NH— moiety, that nitrogen may be optionally substituted by one or more R²³;

R¹⁸, R¹⁹ and R²⁵ are independently selected from halo, hydroxy, cyano, carbamoyl, ureido amino nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

R²⁰, R²¹, R²², R²³ and R²⁶ are independently C₁₋₄alkyl, C₁₋₄alkanoyl, C₁₋₄alkylsulphonyl, sulphamoyl, N—(C₁₋₄alkyl)sulphamoyl, N,N—(C₁₋₄alkyl)₂sulphamoyl, C₁₋₄alkoxycarbonyl, carbamoyl, N—(C₁₋₄alkyl)carbamoyl, N,N—(C₁₋₄alkyl)₂carbamoyl, benzyl, phenethyl, benzoyl, phenylsulphonyl and phenyl;

R²⁴ is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄ alkyl)amino, N,N—(C₁₋₄alkyl)₂amino, C₁₋₄alkanoylamino, N—(C₁₋₄alkyl)carbamoyl, N,N—(C₁₋₄ alkyl)₂carbamoyl, C₁₋₄alkylS(O)ₐ wherein a is 0 to 2, C₁₋₄alkoxycarbonyl, N—(C₁₋₄alkyl)sulphamoyl and N,N—(C₁₋₄alkyl)₂sulphamoyl, carbocyclyl, heterocyclyl; wherein R²⁴ may be independently optionally substituted on carbon by one or more R²⁵; and wherein if said heterocyclyl contains an —NH— moiety, that nitrogen may be optionally substituted by one or more R²⁶;

wherein any saturated cyclic group is a totally or partially saturated, mono or bicyclic ring containing 3-12 atoms of which 0-4 atoms are chosen from nitrogen, sulphur or oxygen, which may be carbon or nitrogen linked;

wherein any heterocyclyl is a saturated, partially saturated or unsaturated, mono or bicyclic ring containing 3-12 atoms of which at least one atom is chosen from nitrogen, sulphur or oxygen, which may be carbon or nitrogen linked, wherein a —CH₂— group can optionally be replaced by a —C(O)— or a ring sulphur atom may be optionally oxidized to form the S-oxides; and

wherein any carbocyclyl is a saturated, partially saturated or unsaturated, mono or bicyclic carbon ring that contains 3-12 atoms, wherein a —CH₂— group can optionally be replaced by a —C(O)—;

or a pharmaceutically acceptable salt thereof.

In some embodiments, a compound of Formula IV is 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N¹—((S)-1-carboxypropyl)carbamoyl]-4-hydroxybenzyl}carbamoylmethoxy)-2,3,4,5-tetrahydro-1,5-benzothiazepine; 1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine; 1,1-dioxo-3,3-dibutyl-5-phenyl-7-methylthio-8-(N-{(R)-α-[N¹—((S)-1-carboxyethyl)carbamoyl]benzyl}carbamoylmethoxy)-2,3,4,5-tetrahydro-1,5-benzothiazepine; or a salt thereof.

In some embodiments, any compound described herein is covalently conjugated to a bile acid using any suitable method. In some embodiments, compounds described herein are covalently bonded to a cyclodextrin or a biodegradable polymer (e.g., a polysaccharide).

In certain embodiments compounds described herein are not systemically absorbed. Moreover, provided herein are compounds that inhibit bile salt recycling in the gastrointestinal tract of an individual. In some embodiments, compounds described herein, may not be transported from the gut lumen and/or do not interact with ASBT. In some embodiments, compounds described herein, do not affect, or minimally affect, fat digestion and/or absorption. In certain embodiments, the administration of a therapeutically effective amount of any compound described herein does not result in gastrointestinal disturbance or lactic acidosis in an individual. In certain embodiments, compounds described herein are administered orally. In some embodiments, an ASBTI is released in the distal ileum. An ASBTI compatible with the methods described herein may be a direct inhibitor, an allosteric inhibitor, or a partial inhibitor of the Apical Sodium-dependent Bile acid Transporter.

In certain embodiments, compounds that inhibit ASBT or any recuperative bile acid transporters are compounds that are described in EP1810689, U.S. Pat. Nos. 6,458,851, 7,413,536, 7,514,421, US Appl. Publication Nos. 20020147184, 20030119809, 20030149010, 20040014806, 20040092500, 20040180861, 20040180860, 20050031651, 20060069080, 20060199797, 20060241121, 20070065428, 20070066644, 20070161578, 20070197628, 20070203183, 20070254952, 20080070888, 20080070892, 20080070889, 20080070984, 20080089858, 20080096921, 20080161400, 20080167356, 20080194598, 20080255202, 20080261990, WO 200250027, WO2005046797, WO2006017257, WO2006105913, WO2006105912, WO2006116499, WO2006117076, WO2006121861, WO2006122186, WO2006124713, WO2007050628, WO2007101531, WO2007134862, WO2007140934, WO2007140894, WO2008028590, WO2008033431, WO2008033464, WO2008031501, WO2008031500, WO2008033465, WO2008034534, WO2008039829, WO2008064788, WO2008064789, WO2008088836, WO2008104306, WO2008124505, and WO2008130616; the compounds described therein that inhibit recuperative bile acid transport are hereby incorporated herein by reference.

In certain embodiments, compounds that inhibit ASBT or any recuperative bile acid transporters are compounds described in WO9316055, WO9418183, WO9418184, WO9605188, WO9608484, WO9616051, WO9733882, WO9838182, WO9935135, WO9840375, WO9964409, WO9964410, WO0001687, WO0047568, WO0061568, DE 19825804, WO0038725, WO0038726, WO0038727 (including those compounds with a 2,3,4,5-tetrahydro-1-benzothiepine 1,1-dioxide structure), WO0038728, WO0166533, WO0250051, EP0864582 (e.g. (3R,5R)-3-butyl-3-ethyl-1,1-dioxido-5-Phenyl-2,3,4,5-tetrahydro-1,4-benzo-thiazepin-8-yl(β-D-glucopyranosiduronic acid, WO9424087, WO9807749, WO9856757, WO9932478, WO9935135, WO0020392, WO0020393, WO0020410, WO0020437, WO0134570, WO0035889, WO0168637, WO0168096, WO0208211, WO03020710, WO03022825, WO03022830, W0030222861, JP10072371, U.S. Pat. Nos. 5,910,494; 5,723,458; 5,817,652; 5,663,165; 5,998,400; 6,465,451, 5,994,391; 6,107,494; 6,387,924; 6,784,201; 6,875,877; 6,740,663; 6,852,753; 5,070,103, 6,114,322, 6,020,330, 7,179,792, EP251315, EP417725, EP489-423, EP549967, EP573848, EP624593, EP624594, EP624595, EP869121, EP1070703, WO04005247, compounds disclosed as having IBAT activity in Drugs of the Future, 24, 425-430 (1999), Journal of Medicinal Chemistry, 48, 5837-5852, (2005) and Current Medicinal Chemistry, 13, 997-1016, (2006); the compounds described therein that inhibit recuperative bile acid transport are hereby incorporated herein by reference.

In some embodiments, compounds that inhibit ASBT or any recuperative bile acid transporter are benzothiepines, benzothiazepines (including 1,2-benzothiazepines; 1,4-benzothiazepines; 1,5-benzothiazepines; and/or 1,2,5-benzothiadiazepines). In some embodiments, compounds that inhibit ASBT or any recuperative bile acid transporter include and are not limited to S-8921 (disclosed in EP597107, WO 9308155), 264W94 (GSK) disclosed in WO 9605188; SC-435 (1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]4-aza-1-azoniabicyclo[2.2.2]octane methanesulfonate salt), SC-635 (Searle); 2164U90 (3-butyl-3-ethyl-2,3,4,5-tetrahydro-5-phenyl-1,4-benzothiazepine 1,1-dioxide); BARI-1741 (Aventis SA), AZD 7508 (Astra Zeneca); barixibat (11-(D-gluconamido)-N-{2-[(1S,2R,3S)-3-hydroxy-3-phenyl-2-(2-pyridyl)-1-(2-pyridylamino)propyl]phenyl}undecanamide) or the like, or combinations thereof. In some embodiments, an ASBTI is:

[chemical structure]

[chemical structure]

[chemical structure]

In certain embodiments, compounds described herein have one or more chiral centers. As such, all stereoisomers are envisioned herein. In various embodiments, compounds described herein are present in optically active or racemic forms. It is to be understood that the compounds of the present invention encompasses racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference for such disclosure). General methods for the preparation of compound as described herein are modified by the use of appropriate reagents and conditions, for the introduction of the various moieties found in the formulae as provided herein. As a guide the following synthetic methods are utilized.

Formation of Covalent Linkages by Reaction of an Electrophile with a Nucleophile

The compounds described herein are modified using various electrophiles and/or nucleophiles to form new functional groups or substituents. Table A entitled “Examples of Covalent Linkages and Precursors Thereof” lists selected non-limiting examples of covalent linkages and precursor functional groups which yield the covalent linkages. Table A is used as guidance toward the variety of electrophiles and nucleophiles combinations available that provide covalent linkages. Precursor functional groups are shown as electrophilic groups and nucleophilic groups.

TABLE A
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it is necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. In some embodiments it is contemplated that each protective group be removable by a different means. Protective groups that are cleaved under totally disparate reaction conditions fulfill the requirement of differential removal.

In some embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl in the presence of amines blocked with acid labile groups such as t-butyl carbamate or with carbamates that are both acid and base stable but hydrolytically removable.

In some embodiments carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a Pd⁰-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups are selected from:

[chemical structure]

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

In some embodiments, ASBTIs described herein are synthesized as described in, for example, WO 9605188, U.S. Pat. Nos. 5,994,391; 7,238,684; 6,906,058; 6,020,330; and 6,114,322. In some embodiments, ASBTIs described herein are synthesized starting from compounds that are available from commercial sources or that are prepared using procedures outlined herein. In some embodiments, compounds described herein are prepared according to the process set forth in Scheme 1:

[chemical structure]

In certain embodiments, the synthesis begins with a reaction of 1,4-diazabicyclo[2.2.2]octane with 4-iodo-1-chloro butane to provide a compound of structure 1-I. Such compounds are prepared in any suitable manner, e.g., as set forth in Tremont, S. J. et. al., J. Med. Chem. 2005, 48, 5837-5852. The compound of structure 1-I is then subjected to a reaction with phenethylamine to provide a compound of structure 1-II. The compound of structure 1-II is then allowed to react with dicyanodiamide to provide a compound of Formula I.

In some embodiments, a first compound of Formula III is subjected to a further reaction to provide a second compound of Formula III as shown in Scheme 2 below.

[chemical structure]

A first compound of Formula III, 1-IA, is alkylated with iodomethane to provide a second compound of Formula III, 1-IB. Alkylation of 1-IB with a compound of structure 2-II provides a further compound of Formula III, IC. In an alternative embodiment, a first compound of Formula III, 1-IA, is alkylated with a compound of structure 2-I to provide a second compound of Formula III, 1-IC.

In some embodiments, compounds described herein are prepared according to the process set forth in Scheme 3:

[chemical structure]

[chemical structure]

[chemical structure]

### GENERAL DEFINITIONS

The term “bile acid,” as used herein, includes steroid acids (and/or the carboxylate anion thereof), and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate, chenodeoxycholic acid, ursodeoxycholic acid, ursodiol, a tauroursodeoxycholic acid, a glycoursodeoxycholic acid, a 7-B-methyl cholic acid, a methyl lithocholic acid, chenodeoxycholate, lithocholic acid, lithocolate, and the like. Taurocholic acid and/or taurocholate are referred to herein as TCA. Any reference to a bile acid used herein includes reference to a bile acid, one and only one bile acid, one or more bile acids, or to at least one bile acid. Therefore, the terms “bile acid,” “bile salt,” “bile acidsalt,” “bile acids,” “bile salts,” and “bile acidssalts” are, unless otherwise indicated, utilized interchangeably herein. Any reference to a bile acid used herein includes reference to a bile acid or a salt thereof. Furthermore, pharmaceutically acceptable bile acid esters are optionally utilized as the “bile acids” described herein, e.g., bile acidssalts conjugated to an amino acid (e.g., glycine or taurine). Other bile acid esters include, e.g., substituted or unsubstituted alkyl ester, substituted or unsubstituted heteroalkyl esters, substituted or unsubstituted aryl esters, substituted or unsubstituted heteroaryl esters, or the like. For example, the term “bile acid” includes cholic acid conjugated with either glycine or taurine: glycocholate and taurocholate, respectively (and salts thereof). Any reference to a bile acid used herein includes reference to an identical compound naturally or synthetically prepared. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted.

The term “subject”, “patient” or “individual” are used interchangeably herein and refer to mammals and non-mammals, e.g., suffering from a disorder described herein. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “about,” as used herein, includes any value that is within 10% of the described value.

The term “between,” as used herein, is inclusive of the lower and upper number of the range.

The term “colon,” as used herein, includes the cecum, ascending colon, hepatic flexure, splenic flexure, descending colon, and sigmoid.

The term “composition,” as used herein includes the disclosure of both a composition and a composition administered in a method as described herein. Furthermore, in some embodiments, the composition of the present invention is or comprises a “formulation,” an oral dosage form or a rectal dosage form as described herein.

The terms “treat,” “treating” or “treatment,” and other grammatical equivalents as used herein, include alleviating, inhibiting or reducing symptoms, reducing or inhibiting severity of, reducing incidence of, reducing or inhibiting recurrence of, delaying onset of, delaying recurrence of, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

The term “non-systemic” or “minimally absorbed” as used herein refers to low systemic bioavailability and/or absorption of an administered compound. In some instances a non-systemic compound is a compound that is substantially not absorbed systemically. In some embodiments, ASBTI compositions described herein deliver the ASBTI to the distal ileum, colon, and/or rectum and not systemically (e.g., a substantial portion of the ASBTI is not systemically absorbed. In some embodiments, the systemic absorption of a non-systemic compound is <0.1%, <0.3%, <0.5%, <0.6%, <0.7%, <0.8%, <0.9%, <1%, <1.5%, <2%, <3%, or <5% of the administered dose (wt. % or mol %). In some embodiments, the systemic absorption of a non-systemic compound is <10% of the administered dose. In some embodiments, the systemic absorption of a non-systemic compound is <15% of the administered dose. In some embodiments, the systemic absorption of a non-systemic compound is <25% of the administered dose. In an alternative approach, a non-systemic ASBTI is a compound that has lower systemic bioavailability relative to the systemic bioavailability of a systemic ASBTI (e.g., compound 100A, 100C). In some embodiments, the bioavailability of a non-systemic ASBTI described herein is <30%, <40%, <50%, <60%, or <70% of the bioavailability of a systemic ASBTI (e.g., compound 100A, 100C).

In another alternative approach, the compositions described herein are formulated to deliver <10% of the administered dose of the ASBTI systemically. In some embodiments, the compositions described herein are formulated to deliver <20% of the administered dose of the ASBTI systemically. In some embodiments, the compositions described herein are formulated to deliver <30% of the administered dose of the ASBTI systemically. In some embodiments, the compositions described herein are formulated to deliver <40% of the administered dose of the ASBTI systemically. In some embodiments, the compositions described herein are formulated to deliver <50% of the administered dose of the ASBTI systemically. In some embodiments, the compositions described herein are formulated to deliver <60% of the administered dose of the ASBTI systemically. In some embodiments, the compositions described herein are formulated to deliver <70% of the administered dose of the ASBTI systemically. In some embodiments, systemic absorption is determined in any suitable manner, including the total circulating amount, the amount cleared after administration, or the like.

The term “ASBT inhibitor” refers to a compound that inhibits apical sodium-dependent bile transport or any recuperative bile salt transport. The term Apical Sodium-dependent Bile Transporter (ASBT) is used interchangeably with the term Ileal Bile Acid Transporter (IBAT).

The term “enhancing enteroendocrine peptide secretion” refers to a sufficient increase in the level of the enteroendocrine peptide agent, for example, to treat any disease or disorder described herein. In some embodiments, enhanced enteroendocrine peptide secretion reverses or alleviates symptoms of Barrett's esophagus or GERD.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

The term “optionally substituted” or “substituted” means that the referenced group substituted with one or more additional group(s). In certain embodiments, the one or more additional group(s) are individually and independently selected from amide, ester, alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “lower alkyl” is a C₁-C₆ alkyl. A “heteroalkyl” group substitutes any one of the carbons of the alkyl group with a heteroatom having the appropriate number of hydrogen atoms attached (e.g., a CH₂ group to an NH group or an O group).

The term “alkylene” refers to a divalent alkyl radical. Any of the above mentioned monovalent alkyl groups may be an alkylene by abstraction of a second hydrogen atom from the alkyl. In one aspect, an alkelene is a C₁-C₁₀alkylene. In another aspect, an alkylene is a C₁-C₆alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, —CH₂CH₂CH₂CH₂CH₂—, —CH₂CH₂CH₂CH₂CH₂CH₂—, and the like.

An “alkoxy” group refers to a (alkyl)O— group, where alkyl is as defined herein.

The term “alkylamine” refers to the N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula C(═O)OR, where R is selected from the group consisting of alkyl, cycloalkyl, aryl, heteroaryl and heteroalicyclic.

As used herein, the term “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl.

The term “aromatic” refers to a planar ring having a delocalized π-electron system containing 4n+2 it electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

[chemical structure]

and the like. Monocyclic cycloalkyls include, but are not limited to, cyclopropyl, cyclobutyl, cyclopentyl, cyclohexyl, cycloheptyl, and cyclooctyl.

The term “heterocyclo” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent 0 or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic. Illustrative examples of heteroaryl groups include the following moieties:

[chemical structure]

and the like.

A “heteroalicyclic” group or “heterocyclo” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocyclo groups, also referred to as non-aromatic heterocycles, include:

[chemical structure]

and the like. The term heteroalicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different. The terms “fluoroalkyl” and “fluoroalkoxy” include haloalkyl and haloalkoxy groups, respectively, in which the halo is fluorine.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and CH═CH—N(CH₃)—CH₃. In some embodiments, up to two heteroatoms are consecutive, such as, by way of example, —CH₂—NH—OCH₃ and CH₂—O—Si(CH₃)₃.

A “cyano” group refers to a —CN group.

An “isocyanato” group refers to a —NCO group.

A “thiocyanato” group refers to a —CNS group.

An “isothiocyanato” group refers to a —NCS group.

“Alkoyloxy” refers to a RC(═O)O— group.

“Alkoyl” refers to a RC(═O)— group.

The term “modulate,” as used herein refers to having some affect on (e.g., increasing, enhancing or maintaining a certain level).

The term “optionally substituted” or “substituted” means that the referenced group may be substituted with one or more additional group(s) individually and independently selected from C₁-C₆alkyl, C₃-C₈cycloalkyl, aryl, heteroaryl, C₂-C₆heteroalicyclic, hydroxy, C₁-C₆alkoxy, aryloxy, arylalkoxy, aralkyloxy, arylalkyloxy, C₁-C₆alkylthio, arylthio, C₁-C₆alkylsulfoxide, arylsulfoxide, C₁-C₆alkylsulfone, arylsulfone, cyano, halo, C₂-C₈acyl, C₂-C₈acyloxy, nitro, C₁-C₆haloalkyl, C₁-C₆fluoroalkyl, and amino, including C₁-C₆alkylamino, and the protected derivatives thereof. By way of example, an optional substituents may be LₛR^{s}, wherein each L^{s} is independently selected from a bond, —O—, —C(═O)—, —S—, —S(═O)—, —S(═O)₂—, —NH—, —NHC(═O)—, —C(═O)NH—, S(═O)₂NH—, —NHS(═O)₂—, —OC(═O)NH—, —NHC(═O)O—, —(C₁-C₆alkyl)-, or —(C₂-C₆alkenyl)-; and each R^{s} is independently selected from H, (C₁-C₄alkyl), (C₃-C₈cycloalkyl), heteroaryl, aryl, and C₁-C₆heteroalkyl. Optionally substituted non-aromatic groups may be substituted with one or more oxo (═O). The protecting groups that may form the protective derivatives of the above substituents are known to those of skill in the art and may be found in references such as Greene and Wuts, above. In some embodiments, alkyl groups described herein are optionally substituted with an O that is connected to two adjacent carbon atoms (i.e., forming an epoxide).

The term “therapeutically effective amount” or an “effective amount” as used herein, refers to a sufficient amount of a therapeutically active agent to provide a desired effect in a subject or individual. In some embodiments, a “therapeutically effective amount” or an “effective amount” of an ASBTI refers to a sufficient amount of an ASBTI to treat Barrett's esophagus or GERD in a subject or individual.

### L-Cells

Inventors have discovered that enteroendocrine L-cells play a role in repair. The epithelial barrier is also a key component in host defense. A further pre-proglucagon splice product, GLP-2, is secreted by enteroendocrine L-cells in the distal small intestine and has been shown to improve intestinal wound healing in a TGF-B (anti-inflammatory cytokine TGF-B), mediated process, small bowel responding better than large bowel. GLP-2 has also been shown to ameliorate the barrier dysfunction induced by experimental stress and food allergy. Again, L-cells are activated by luminal nutrients, and the barrier compromise observed in TPN may partly reflect its hyposecretion in the absence of enteral stimuli. Moreover, GLP-2 is also responsible, at least in part for growth and adaptation observed in short-bowel models. Therefore, abnormal enteroendocrine cells (EEC) function may predispose to GI inflammatory disorders, and the underlying nutrient-EEC-vagal pathways are targets in the injured gut as contemplated in the present embodiments.

L-cells are scattered throughout the epithelial layer of the gut from the duodenum to the rectum, with the highest numbers occurring in the ileum, colon, and rectum. They are characterized by an open-cell morphology, with apical microvilli facing into the gut lumen and secretory vesicles located adjacent to the basolateral membrane, and are therefore in direct contact with nutrients in the intestinal lumen. Furthermore, L-cells are located in close proximity to both neurons and the microvasculature of the intestine, thereby allowing the L-cell to be affected by both neural and hormonal signals. As well as Glucagon-Like Peptide 1 (GLP-1) and Glucagon-Like Peptide 2 (GLP-2), L-cells also secrete peptide YY (PYY), and glutamate. The cells are just one member of a much larger family of enteroendocrine cells that secrete a range of hormones, including ghrelin, GIP, cholecystokinin, somatostatin, and secretin, which are involved in the local coordination of gut physiology, as well as in playing wider roles in the control of cytokine release and/or controlling the adaptive process, attenuating intestinal injury, reducing bacterial translocation, inhibiting the release of free radical oxygen, or any combination thereof. L-cells are unevenly distributed in the gastrointestinal tract, within higher concentrations in the distal portion of the gastrointestinal tract (e.g., in the distal ileum, colon and rectum).

### Bile Acid

Bile contains water, electrolytes and a numerous organic molecules including bile acids, cholesterol, phospholipids and bilirubin. Bile is secreted from the liver and stored in the gall bladder, and upon gall bladder contraction, due to ingestion of a fatty meal, bile passes through the bile duct into the intestine. Bile acidssalts are critical for digestion and absorption of fats and fat-soluble vitamins in the small intestine. Adult humans produce 400 to 800 mL of bile daily. The secretion of bile can be considered to occur in two stages. Initially, hepatocytes secrete bile into canaliculi, from which it flows into bile ducts and this hepatic bile contains large quantities of bile acids, cholesterol and other organic molecules. Then, as bile flows through the bile ducts, it is modified by addition of a watery, bicarbonate-rich secretion from ductal epithelial cells. Bile is concentrated, typically five-fold, during storage in the gall bladder.

The flow of bile is lowest during fasting, and a majority of that is diverted into the gallbladder for concentration. When chyme from an ingested meal enters the small intestine, acid and partially digested fats and proteins stimulate secretion of cholecystokinin and secretin, both of which are important for secretion and flow of bile. Cholecystokinin (cholecysto=gallbladder and kinin=movement) is a hormone which stimulates contractions of the gallbladder and common bile duct, resulting in delivery of bile into the gut. The most potent stimulus for release of cholecystokinin is the presence of fat in the duodenum. Secretin is a hormone secreted in response to acid in the duodenum, and it simulates biliary duct cells to secrete bicarbonate and water, which expands the volume of bile and increases its flow out into the intestine.

Bile acidssalts are derivatives of cholesterol. Cholesterol, ingested as part of the diet or derived from hepatic synthesis, are converted into bile acidssalts in the hepatocyte. Examples of such bile acidssalts include cholic and chenodeoxycholic acids, which are then conjugated to an amino acid (such as glycine or taurine) to yield the conjugated form that is actively secreted into canaliculi. The most abundant of the bile salts in humans are cholate and deoxycholate, and they are normally conjugated with either glycine or taurine to give glycocholate or taurocholate respectively.

Free cholesterol is virtually insoluble in aqueous solutions, however in bile it is made soluble by the presence of bile acidssalts and lipids. Hepatic synthesis of bile acidssalts accounts for the majority of cholesterol breakdown in the body. In humans, roughly 500 mg of cholesterol are converted to bile acidssalts and eliminated in bile every day. Therefore, secretion into bile is a major route for elimination of cholesterol. Large amounts of bile acidssalts are secreted into the intestine every day, but only relatively small quantities are lost from the body. This is because approximately 95% of the bile acidssalts delivered to the duodenum are absorbed back into blood within the ileum, by a process is known as “Enterohepatic Recirculation”.

Venous blood from the ileum goes straight into the portal vein, and hence through the sinusoids of the liver. Hepatocytes extract bile acidssalts very efficiently from sinusoidal blood, and little escapes the healthy liver into systemic circulation. Bile acidssalts are then transported across the hepatocytes to be resecreted into canaliculi. The net effect of this enterohepatic recirculation is that each bile salt molecule is reused about 20 times, often two or three times during a single digestive phase. Bile biosynthesis represents the major metabolic fate of cholesterol, accounting for more than half of the approximate 800 mg/day of cholesterol that an average adult uses up in metabolic processes. In comparison, steroid hormone biosynthesis consumes only about 50 mg of cholesterol per day. Much more that 400 mg of bile salts is required and secreted into the intestine per day, and this is achieved by re-cycling the bile salts. Most of the bile salts secreted into the upper region of the small intestine are absorbed along with the dietary lipids that they emulsified at the lower end of the small intestine. They are separated from the dietary lipid and returned to the liver for re-use. Re-cycling thus enables 20-30 g of bile salts to be secreted into the small intestine each day.

Bile acidssalts are amphipathic, with the cholesterol-derived portion containing both hydrophobic (lipid soluble) and polar (hydrophilic) moieties while the amino acid conjugate is generally polar and hydrophilic. This amphipathic nature enables bile acidssalts to carry out two important functions: emulsification of lipid aggregates and solubilization and transport of lipids in an aqueous environment. Bile acidssalts have detergent action on particles of dietary fat which causes fat globules to break down or to be emulsified. Emulsification is important since it greatly increases the surface area of fat available for digestion by lipases which cannot access the inside of lipid droplets. Furthermore, bile acidssalts are lipid carriers and are able to solubilize many lipids by forming micelles and are critical for transport and absorption of the fat-soluble vitamins.

### Pharmaceutical Compositions and Methods of Use

In some embodiments, compositions described herein are administered for delivery of enteroendocrine peptide secretion enhancing agents to a subject or individual. In certain embodiments, any compositions described herein are formulated for ileal, rectal and/or colonic delivery. In more specific embodiments, the composition is formulated for non-systemic or local delivery to the rectum and/or colon. It is to be understood that as used herein, delivery to the colon includes delivery to sigmoid colon, transverse colon, and/or ascending colon. In still more specific embodiments, the composition is formulated for non-systemic or local delivery to the rectum and/or colon is administered rectally. In other specific embodiments, the composition is formulated for non-systemic or local delivery to the rectum and/or colon is administered orally.

In some embodiments, provided herein is a composition comprising an enteroendocrine peptide secretion enhancing agent and, optionally, a pharmaceutically acceptable carrier for alleviating symptoms of Barrett's esophagus or GERD in an individual.

In certain embodiments, the composition comprises an enteroendocrine peptide secretion enhancing agent and an absorption inhibitor. In specific embodiments, the absorption inhibitor is an inhibitor that inhibits the absorption of the (or at least one of the) specific enteroendocrine peptide secretion enhancing agent with which it is combined. In some embodiments, the composition comprises an enteroendocrine peptide secretion enhancing agent, an absorption inhibitor and a carrier (e.g., an orally suitable carrier or a rectally suitable carrier, depending on the mode of intended administration). In certain embodiments, the composition comprises an enteroendocrine peptide secretion enhancing agent, an absorption inhibitor, a carrier, and one or more of a cholesterol absorption inhibitor, an enteroendocrine peptide, a peptidase inhibitor, a spreading agent, and a wetting agent.

In other embodiments, the compositions described herein are administered orally for non-systemic delivery of the bile salt active component to the rectum and/or colon, including the sigmoid colon, transverse colon, and/or ascending colon. In specific embodiments, compositions formulated for oral administration are, by way of non-limiting example, enterically coated or formulated oral dosage forms, such as, tablets and/or capsules. It is to be understood that the terms “subject” and “individual” are utilized interchangeably herein and include, e.g., humans and human patients in need of treatment.

### Absorption Inhibitors

In certain embodiments, the composition described herein as being formulated for the non-systemic delivery of ASBTI further includes an absorption inhibitor. As used herein, an absorption inhibitor includes an agent or group of agents that inhibit absorption of a bile acidsalt.

Suitable bile acid absorption inhibitors (also described herein as absorption inhibiting agents) include, by way of non-limiting example, anionic exchange matrices, polyamines, quaternary amine containing polymers, quaternary ammonium salts, polyallylamine polymers and copolymers, colesevelam, colesevelam hydrochloride, CholestaGel (N,N,N-trimethyl-6-(2-propenylamino)-1-hexanaminium chloride polymer with (chloromethyl)oxirane, 2-propen-1-amine and N-2-propenyl-1-decanamine hydrochloride), cyclodextrins, chitosan, chitosan derivatives, carbohydrates which bind bile acids, lipids which bind bile acids, proteins and proteinaceous materials which bind bile acids, and antibodies and albumins which bind bile acids. Suitable cyclodextrins include those that bind bile acidssalts such as, by way of non-limiting example, β-cyclodextrin and hydroxypropyl-β-cyclodextrin. Suitable proteins, include those that bind bile acidssalts such as, by way of non-limiting example, bovine serum albumin, egg albumin, casein, α^{□}-acid glycoprotein, gelatin, soy proteins, peanut proteins, almond proteins, and wheat vegetable proteins.

In certain embodiments the absorption inhibitor is cholestyramine. In specific embodiments, cholestyramine is combined with a bile acid. Cholestyramine, an ion exchange resin, is a styrene polymer containing quaternary ammonium groups crosslinked by divinylbenzene. In other embodiments, the absorption inhibitor is colestipol. In specific embodiments, colestipol is combined with a bile acid. Colestipol, an ion exchange resin, is a copolymer of diethylenetriamine and 1-chloro-2,3-epoxypropane.

In certain embodiments of the compositions and methods described herein the ASBTI is linked to an absorption inhibitor, while in other embodiments the ASBTI and the absorption inhibitor are separate molecular entities.

### Cholesterol Absorption Inhibitors

In certain embodiments, a composition described herein optionally includes at least one cholesterol absorption inhibitor. Suitable cholesterol absorption inhibitors include, by way of non-limiting example, ezetimibe (SCH 58235), ezetimibe analogs, ACT inhibitors, stigmastanyl phosphorylcholine, stigmastanyl phosphorylcholine analogues, β-lactam cholesterol absorption inhibitors, sulfate polysaccharides, neomycin, plant saponins, plant sterols, phytostanol preparation FM-VP4, Sitostanol, β-sitosterol, acyl-CoA:cholesterol-O-acyltransferase (ACAT) inhibitors, Avasimibe, Implitapide, steroidal glycosides and the like. Suitable ezetimibe analogs include, by way of non-limiting example, SCH 48461, SCH 58053 and the like. Suitable ACT inhibitors include, by way of non-limiting example, trimethoxy fatty acid anilides such as Cl-976, 3-[decyldimethylsilyl]-N-[2-(4-methylphenyl)-1-phenylethyl]-propanamide, melinamide and the like. β-lactam cholesterol absorption inhibitors include, by way of non-limiting example, (3R-4S)-1,4-bis-(4-methoxyphenyl)-3-(3-phenylpropyl)-2-azetidinone and the like.

### Peptidase Inhibitors

In some embodiments, the compositions described herein optionally include at least one peptidase inhibitor. Such peptidase inhibitors include, but are not limited to, dipeptidyl peptidase-4 inhibitors (DPP-4), neutral endopeptidase inhibitors, and converting enzyme inhibitors. Suitable dipeptidyl peptidase-4 inhibitors (DPP-4) include, by way of non-limiting example, Vildaglipti, 2S)-1-{2-[(3-hydroxy-1-adamantyl)amino]acetyl}pyrrolidine-2-carbonitrile, Sitagliptin, (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one, Saxagliptin, and (1S,3S,5S)-2-[(2S)-2-amino-2-(3-hydroxy-1-adamantyl)acetyl]-2-azabicyclo[3.1.0]hexane-3-carbonitrile. Such neutral endopeptidase inhibitors include, but are not limited to, Candoxatrilat and Ecadotril.

### Spreading Agents/Wetting Agents

In certain embodiments, the composition described herein optionally comprises a spreading agent. In some embodiments, a spreading agent is utilized to improve spreading of the composition in the colon and/or rectum. Suitable spreading agents include, by way of non-limiting example, hydroxyethylcellulose, hydroxypropymethyl cellulose, polyethylene glycol, colloidal silicon dioxide, propylene glycol, cyclodextrins, microcrystalline cellulose, polyvinylpyrrolidone, polyoxyethylated glycerides, polycarbophil, di-n-octyl ethers, Cetiol™OE, fatty alcohol polyalkylene glycol ethers, Aethoxal™B), 2-ethylhexyl palmitate, Cegesoft™C 24), and isopropyl fatty acid esters.

In some embodiments, the compositions described herein optionally comprise a wetting agent. In some embodiments, a wetting agent is utilized to improve wettability of the composition in the colon and rectum. Suitable wetting agents include, by way of non-limiting example, surfactants. In some embodiments, surfactants are selected from, by way of non-limiting example, polysorbate (e.g., 20 or 80), stearyl hetanoate, capryliccapric fatty acid esters of saturated fatty alcohols of chain length C₁₂-C₁₈, isostearyl diglycerol isostearic acid, sodium dodecyl sulphate, isopropyl myristate, isopropyl palmitate, and isopropyl myristateisopropyl stearateisopropyl palmitate mixture.

### Vitamins

In some embodiments, the methods provided herein further comprise administering one or more vitamins.

In some embodiments, the vitamin is vitamin A, B1, B2, B3, B5, B6, B7, B9, B12, C, D, E, K, folic acid, pantothenic acid, niacin, riboflavin, thiamine, retinol, beta carotene, pyridoxine, ascorbic acid, cholecalciferol, cyanocobalamin, tocopherols, phylloquinone, menaquinone.

In some embodiments, the vitamin is a fat soluble vitamin such as vitamin A, D, E, K, retinol, beta carotene, cholecalciferol, tocopherols, phylloquinone. In a preferred embodiment, the fat soluble vitamin is tocopherol polyethylene glycol succinate (TPGS).

### Bile Acid Sequestrants/Binders

In some embodiments, a labile bile acid sequestrant is an enzyme dependent bile acid sequestrant. In certain embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, 13-D-glucosidase, α-L-arabinofuranosidase, 13-D-xylopyranosidase or the like.

In certain embodiments, a labile bile acid sequestrant is a time dependent bile acid sequestrant. In some embodiments, a labile bile acid sequestrant releases a bile acid or is degraded after 1, 2, 3, 4, 5, 6, 7, 8, 9, or 10 seconds of sequestration. In some embodiments, a labile bile acid sequestrant releases a bile acid or is degraded after 15, 20, 25, 30, 35, 40, 45, 50, or 55 seconds of sequestration. In some embodiments, a labile bile acid sequestrant releases a bile acid or is degraded after 1, 2, 3, 4, 5, 6, 7, 8, 9, or 10 minutes of sequestration. In some embodiments, a labile bile acid sequestrant releases a bile acid or is degraded after about 15, 20, 25, 30, 35, 45, 50, or 55 minutes of sequestration. In some embodiments, a labile bile acid sequestrant releases a bile acid or is degraded after about 1, 2, 3, 4, 5, 6, 7, 8, 9, 10, 11, 12, 13, 14, 15, 16, 17, 18, 19, 20, 21, 22, 23, or 24 hours of sequestration. In some embodiments, a labile bile acid sequestrant releases a bile acid or is degraded after 1, 2, or 3 days of sequestration.

In some embodiments, the labile bile acid sequestrant has a low affinity for bile acid. In certain embodiments, the labile bile acid sequestrant has a high affinity for a primary bile acid and a low affinity for a secondary bile acid.

In some embodiments, the labile bile acid sequestrant is a pH dependent bile acid sequestrant. In certain embodiments, the pH dependent bile acid sequestrant has a high affinity for bile acid at a pH of 6 or below and a low affinity for bile acid at a pH above 6. In certain embodiments, the pH dependent bile acid sequestrant has a high affinity for bile acid at a pH of 6.5 or below and a low affinity for bile acid at a pH above 6.5. In certain embodiments, the pH dependent bile acid sequestrant has a high affinity for bile acid at a pH of 7 or below and a low affinity for bile acid at a pH above 7. In certain embodiments, the pH dependent bile acid sequestrant has a high affinity for bile acid at a pH of 7.1 or below and a low affinity for bile acid at a pH above 7.1. In certain embodiments, the pH dependent bile acid sequestrant has a high affinity for bile acid at a pH of 7.2 or below and a low affinity for bile acid at a pH above 7.2. In certain embodiments, the pH dependent bile acid sequestrant has a high affinity for bile acid at a pH of 7.3 or below and a low affinity for bile acid at a pH above 7.3. In certain embodiments, the pH dependent bile acid sequestrant has a high affinity for bile acid at a pH of 7.4 or below and a low affinity for bile acid at a pH above 7.4. In certain embodiments, the pH dependent bile acid sequestrant has a high affinity for bile acid at a pH of 7.5 or below and a low affinity for bile acid at a pH above 7.5. In certain embodiments, the pH dependent bile acid sequestrant has a high affinity for bile acid at a pH of 7.6 or below and a low affinity for bile acid at a pH above 7.6. In certain embodiments, the pH dependent bile acid sequestrant has a high affinity for bile acid at a pH of 7.7 or below and a low affinity for bile acid at a pH above 7.7. In certain embodiments, the pH dependent bile acid sequestrant has a high affinity for bile acid at a pH of 7.8 or below and a low affinity for bile acid at a pH above 7.8. In some embodiments, the pH dependent bile acid sequestrant degrades at a pH above 6. In some embodiments, the pH dependent bile acid sequestrant degrades at a pH above 6.5. In some embodiments, the pH dependent bile acid sequestrant degrades at a pH above 7. In some embodiments, the pH dependent bile acid sequestrant degrades at a pH above 7.1. In some embodiments, the pH dependent bile acid sequestrant degrades at a pH above 7.2. In some embodiments, the pH dependent bile acid sequestrant degrades at a pH above 7.3. In some embodiments, the pH dependent bile acid sequestrant degrades at a pH above 7.4. In some embodiments, the pH dependent bile acid sequestrant degrades at a pH above 7.5. In some embodiments, the pH dependent bile acid sequestrant degrades at a pH above 7.6. In some embodiments, the pH dependent bile acid sequestrant degrades at a pH above 7.7. In some embodiments, the pH dependent bile acid sequestrant degrades at a pH above 7.8. In some embodiments, the pH dependent bile acid sequestrant degrades at a pH above 7.9.

In certain embodiments, the labile bile acid sequestrant is lignin or a modified lignin. In some embodiments, the labile bile acid sequestrant is a polycationic polymer or copolymer. In certain embodiments, the labile bile acid sequestrant is a polymer or copolymer comprising one or more N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof.

In some embodiments, the bile acid binder is cholestyramine, and various compositions including cholestyramine, which are described, for example, in U.S. Pat. Nos. 3,383,281; 3,308, 020; 3,769, 399; 3,846, 541; 3,974, 272; 4,172, 120; 4,252, 790; 4,340, 585; 4,814, 354; 4,874, 744; 4,895, 723; 5,695, 749; and 6,066, 336. In some embodiments, the bile acid binder is colestipol or colesevelam.

### Methods

Provided herein, in certain embodiments, are methods for treating Barrett's esophagus or GERD comprising non-systemic administration of a therapeutically effective amount of an ASBTI. Provided herein, in certain embodiments, are methods for treating Barrett's esophagus or GERD comprising contacting the gastrointestinal tract of an individual in need thereof with an ASBTI. Also provided herein are methods for reducing intraenterocyte bile acids, reducing damage to hepatocellular or intestinal architecture caused by Barrett's esophagus or GERD, of an individual comprising administration of a therapeutically effective amount of an ASBTI to an individual in need thereof.

In some embodiments, provided herein is a method of treating Barrett's esophagus in an individual comprising administering a therapeutically effective amount of any ASBTI described herein. Provided herein are methods for reducing damage to esophageal or intestinal architecture or cells from Barrett's esophagus comprising administration of a therapeutically effective amount of an ASBTI. In certain embodiments, provided herein are methods for reducing intraenterocyte bile acidssalts comprising administration of a therapeutically effective amount of an ASBTI to an individual in need thereof.

In some embodiments, provided herein are methods for treating Barrett's esophagus or GERD consisting essentially of non-systemic administration of a therapeutically effective amount of an ASBTI. Provided herein, in certain embodiments, are methods for treating Barrett's esophagus or GERD consisting essentially of contacting the gastrointestinal tract of an individual in need thereof with an ASBTI. Also provided herein are methods for reducing intraenterocyte bile acids, reducing damage to hepatocellular or intestinal architecture caused by Barrett's esophagus or GERD, of an individual consisting essentially of administration of a therapeutically effective amount of an ASBTI to an individual in need thereof.

In some embodiments, provided herein is a method of treating Barrett's esophagus in an individual consisting essentially of administering a therapeutically effective amount of any ASBTI described herein. Provided herein are methods for reducing damage to esophageal or intestinal architecture or cells from Barrett's esophagus consisting essentially of administration of a therapeutically effective amount of an ASBTI. In certain embodiments, provided herein are methods for reducing intraenterocyte bile acidssalts consisting essentially of administration of a therapeutically effective amount of an ASBTI to an individual in need thereof.

In some embodiments, the methods provide for inhibition of bile salt recycling upon administration of any of the compounds described herein to an individual. In some embodiments, an ASBTI described herein is systemically absorbed upon administration. In some embodiments, an ASBTI described herein is not absorbed systemically. In some embodiments, an ASBTI herein is administered to the individual orally. In some embodiments, an ASBTI described herein is delivered and/or released in the distal gastrointestinal tract of an individual.

In certain instances, contacting the distal ileum of an individual with an ASBTI (e.g., any ASBTI described herein) inhibits bile acid reuptake and increases the concentration of bile acidssalts in the vicinity of L-cells in the distal ileum and/or colon and/or rectum, thereby reducing intraenterocyte bile acids, reducing serum and/or hepatic bile acid levels, reducing overall bile acid load, and/or reducing damage to esophageal or intestinal architecture caused by Barrett's esophagus or GERD. Without being limited to any particular theory, reducing serum and/or hepatic bile acid levels ameliorates Barrett's esophagus or GERD.

Administration of a compound described herein is achieved in any suitable manner including, by way of non-limiting example, by oral, enteric, parenteral (e.g., intravenous, subcutaneous, intramuscular), intranasal, buccal, topical, rectal, or transdermal administration routes. Any compound or composition described herein is administered in a method or formulation appropriate to treat a new born or an infant. Any compound or composition described herein is administered in an oral formulation (e.g., solid or liquid) to treat a new born or an infant. Any compound or composition described herein is administered prior to ingestion of food, with food or after ingestion of food.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein are administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In some embodiments, doses administered include those up to the maximum tolerable dose by a newborn or an infant.

In certain embodiments, about 0.001-5000 mg per day, from about 0.001-1500 mg per day, about 0.001 to about 100 mg/day, about 0.001 to about 50 mg/day, or about 0.001 to about 30 mg/day, or about 0.001 to about 10 mg/day of a compound described herein is administered to an individual in need thereof. In various embodiments, the desired dose is conveniently presented in a single dose or in divided doses administered simultaneously (or over a short period of time) or at appropriate intervals, for example as two, three, four or more sub-doses per day. In various embodiments, a single dose is from about 0.001 mg/kg to about 500 mg/kg. In various embodiments, a single dose is from about 0.001, 0.01, 0.1, 1, or 10 mg/kg to about 10, 50, 100, or 250 mg/kg. In various embodiments, a single dose of an ASBTI is from about 0.001 mg/kg to about 100 mg/kg. In various embodiments, a single dose of an ASBTI is from about 0.001 mg/kg to about 50 mg/kg. In various embodiments, a single dose of an ASBTI is from about 0.001 mg/kg to about 10 mg/kg. In various embodiments, a single dose of an ASBTI is administered every 6 hours, every 12 hours, every 24 hours, every 48 hours, every 72 hours, every 96 hours, every 5 days, every 6 days, or once a week. In some embodiments the total single dose of an ASBTI is in the range described herein.

In the case wherein the patient's status does improve, upon the doctor's discretion an ASBTI is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%. In some embodiments the total single dose of an ASBTI is in the range described herein.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

In some embodiments, the systemic exposure of a therapeutically effective amount of any non-systemic ASBTI described herein (e.g., LUM001, LUM002, SC-435) is reduced when compared to the systemic exposure of a therapeutically effective amount of any systemically absorbed ASBTI (e.g., Compounds 100A, 100C). In some embodiments, the AUC of a therapeutically effective amount of any non-systemic ASBTI described herein (e.g., LUM001, LUM002, SC-435) is at least 10%, at least 20%, at least 30%, at least 40%, at least 50%, at least 60%, at least 70%, at least 80% or at least 90% reduced when compared to the AUC of any systemically absorbed ASBTI (e.g., Compounds 100A, 100C).

In certain embodiments, the Cmax of a therapeutically effective amount of any non-systemic ASBTI described herein (e.g., LUM001, LUM002, SC-435) is at least 10%, at least 20%, at least 30%, at least 40%, at least 50%, at least 60%, at least 70%, at least 80%, at least 90%, at least 95%, or at least 99% reduced when compared to the Cmax of any systemically absorbed ASBTI (e.g. Compound 100A).

In certain embodiments, the pharmaceutical composition administered includes a therapeutically effective amount of a bile salt, a bile acid mimic, or a bile salt mimic, an absorption inhibitor and a carrier (e.g., an orally suitable carrier or a rectally suitable carrier, depending on the mode of intended administration). In certain embodiments, the pharmaceutical composition used or administered comprises a bile salt, a bile acid mimic, or a bile salt mimic, an absorption inhibitor, a carrier, and one or more of a cholesterol absorption inhibitor, an enteroendocrine peptide, a peptidase inhibitor, a spreading agent, and a wetting agent. In certain embodiments, the pharmaceutical composition administered consists essentially of a therapeutically effective amount of a bile salt, a bile acid mimic, or a bile salt mimic, an absorption inhibitor and a carrier (e.g., an orally suitable carrier or a rectally suitable carrier, depending on the mode of intended administration). In some embodiments, the pharmaceutical composition consists essentially of an ASBTI and a carrier. In some embodiments, the pharmaceutical composition consists essentially of an ASBTI as described herein and a carrier.

In another specific embodiment, the pharmaceutical composition used to prepare an oral dosage form or administered orally comprises a bile salt, a bile acid mimic, or a bile salt mimic, an absorption inhibitor, an orally suitable carrier, an optional cholesterol absorption inhibitor, an optional enteroendocrine peptide, an optional peptidase inhibitor, an optional spreading agent, and an optional wetting agent. In certain embodiments, the orally administered compositions evokes an anorectal response. In specific embodiments, the anorectal response is an increase in secretion of one or more enteroendocrine by cells in the colon and/or rectum (e.g., in L-cells the epithelial layer of the colon and/or rectum). In some embodiments, the anorectal response persists for at least 1, 2, 3, 4,5,6,7,8, 9, 10, 11, 12, 13, 14, 15, 16, 17, 18, 19, 20, 21, 22, 23 or 24 hours. In other embodiments the anorectal response persists for a period between 24 hours and 48 hours, while in other embodiments the anorectal response persists for persists for a period greater than 48 hours.

### Routes of Administration and Dosage

In some embodiments, the compositions described herein and the compositions administered in the methods described herein are formulated to inhibit bile acid reuptake, or reduce serum or hepatic bile acid levels. In certain embodiments, the compositions described herein are formulated for oral administration. In some embodiments, the compositions described herein are formulated for rectal administration. In some embodiments, the compositions described herein are combined with a device for local delivery of the compositions to the rectum and/or colon (sigmoid colon, transverse colon, or ascending colon). In certain embodiments, for rectal administration the composition described herein are formulated as enemas, rectal gels, rectal foams, rectal aerosols, suppositories, jelly suppositories, or retention enemas. In some embodiments, for oral administration the compositions described herein are formulated for oral administration and enteric delivery to the colon.

In certain embodiments, the compositions or methods described herein are non-systemic. In some embodiments, compositions described herein deliver the ASBTI to the gastrointestinal tract and not systemically (e.g., a substantial portion of the enteroendocrine peptide secretion enhancing agent is not systemically absorbed). In some embodiments, oral compositions described herein non-systemically deliver the ASBTI to the gastrointestinal tract. In some embodiments, rectal compositions described herein non-systemically deliver the ASBTI to the jejunum, ileum, colon, and/or rectum. In certain embodiments, non-systemic compositions described herein deliver less than 50% w/w of the ASBTI systemically. In certain embodiments, non-systemic compositions described herein deliver less than 40% w/w of the ASBTI systemically. In certain embodiments, non-systemic compositions described herein deliver less than 30% w/w of the ASBTI systemically. In certain embodiments, non-systemic compositions described herein deliver less than 25% w/w of the ASBTI systemically. In certain embodiments, non-systemic compositions described herein deliver less than 20% w/w of the ASBTI systemically. In certain embodiments, non-systemic compositions described herein deliver less than 15% w/w of the ASBTI systemically. In certain embodiments, non-systemic compositions described herein deliver less than 10% w/w of the ASBTI systemically. In certain embodiments, non-systemic compositions described herein deliver less than 5% w/w of the ASBTI systemically. In certain embodiments, non-systemic compositions described herein deliver less than 1% w/w of the ASBTI systemically. In some embodiments, systemic absorption is determined in any suitable manner, including the total circulating amount, the amount cleared after administration, or the like.

In certain embodiments, the compositions and/or formulations described herein are administered at least once a day. In certain embodiments, the formulations containing the ASBTI are administered at least twice a day, while in other embodiments the formulations containing the ASBTI are administered at least three times a day. In certain embodiments, the formulations containing the ASBTI are administered up to five times a day. It is to be understood that in certain embodiments, the dosage regimen of composition containing the ASBTI described herein to is determined by considering various factors such as the patient's age, sex, and diet.

The concentration of the ASBTI administered in the formulations described herein ranges from about 1 mM to about 1 M. In certain embodiments the concentration of the ASBTI administered in the formulations described herein ranges from about 1 mM to about 750 mM. In certain embodiments the concentration of the ASBTI administered in the formulations described herein ranges from about 1 mM to about 500 mM. In certain embodiments the concentration of the ASBTI administered in the formulations described herein ranges from about 5 mM to about 500 mM. In certain embodiments the concentration of the ASBTI administered in the formulations described herein ranges from about 10 mM to about 500 mM. In certain embodiments the concentration of the administered in the formulations described herein ranges from about 25 mM to about 500 mM. In certain embodiments the concentration of the ASBTI administered in the formulations described herein ranges from about 50 mM to about 500 mM. In certain embodiments the concentration of the ASBTI administered in the formulations described herein ranges from about 100 mM to about 500 mM. In certain embodiments the concentration of the ASBTI administered in the formulations described herein ranges from about 200 mM to about 500 mM.

In certain embodiments, any composition described herein comprises a therapeutically effective amount (e.g., to treat Barrett's esophagus or GERD) of ursodiol. In some embodiments, ursodiol may be substituted for any other therapeutic bile acid or salt. In some embodiments, compositions described herein comprise or methods described herein comprise administering about 0.01 mg to about 10 g of ursodiol. In certain embodiments, a composition described herein comprises or a method described herein comprises administering about 0.1 mg to about 500 mg of ursodiol. In certain embodiments, a composition described herein comprises or a method described herein comprises administering about 0.1 mg to about 100 mg of ursodiol. In certain embodiments, a composition described herein comprises or a method described herein comprises administering about 0.1 mg to about 50 mg of ursodiol. In certain embodiments, a composition described herein comprises or a method described herein comprises administering about 0.1 mg to about 10 mg of ursodiol. In certain embodiments, a composition described herein comprises or a method described herein comprises administering about 0.5 mg to about 10 mg of ursodiol. In some embodiments, compositions described herein comprise or methods described herein comprise administering about 0.1 mmol to about 1 mol of ursodiol. In certain embodiments, a composition described herein comprises or a method described herein comprises administering about 0.01 mmol to about 500 mmol of ursodiol. In certain embodiments, a composition described herein comprises or a method described herein comprises administering about 0.1 mmol to about 100 mmol of ursodiol. In certain embodiments, a composition described herein comprises or a method described herein comprises administering about 0.5 mmol to about 30 mmol of ursodiol. In certain embodiments, a composition described herein comprises or a method described herein comprises administering about 0.5 mmol to about 20 mmol of ursodiol. In certain embodiments, a composition described herein comprises or a method described herein comprises administering about 1 mmol to about 10 mmol of ursodiol. In certain embodiments, a composition described herein comprises or a method described herein comprises administering about 0.01 mmol to about 5 mmol of ursodiol. In certain embodiments, a composition described herein comprises or a method described herein comprises administering about 0.1 mmol to about 1 mmol of ursodiol. In various embodiments, certain bile acidssalts have different potencies and dosing is optionally adjusted accordingly.

In certain embodiments, by targeting the distal gastrointestinal tract (e.g., ileum, colon, and/or rectum), compositions and methods described herein provide efficacy (e.g., in reducing microbial growth and/or alleviating symptoms of Barrett's esophagus or GERD) with a reduced dose of enteroendocrine peptide secretion enhancing agent (e.g., as compared to an oral dose that does not target the distal gastrointestinal tract).

In certain embodiments, liquid carrier vehicles or co-solvents in the compositions and/or formulations described herein include, by way of non-limiting example, purified water, propylene glycol, PEG200, PEG300, PEG400, PEG600, polyethyleneglycol, ethanol, 1-propanol, 2-propanol, 1-propen-3-ol (allyl alcohol), propylene glycol, glycerol, 2-methyl-2-propanol, formamide, methyl formamide, dimethyl formamide, ethyl formamide, diethyl formamide, acetamide, methyl acetamide, dimethyl acetamide, ethyl acetamide, diethyl acetamide, 2-pyrrolidone, N-methyl-2-pyrrolidone, N-ethyl-2-pyrrolidone, tetramethyl urea, 1,3-dimethyl-2-imidazolidinone, propylene carbonate, 1,2-butylene carbonate, 2,3-butylene carbonate, dimethyl sulfoxide, diethyl sulfoxide, hexamethyl phosphoramide, pyruvic aldehyde dimethylacetal, dimethylisosorbide and combinations thereof.

In some embodiments, stabilizers used in compositions and/or formulations described herein include, but are not limited to, partial glycerides of polyoxyethylenic saturated fatty acids.

In certain embodiments, surfactants/emulsifiers used in the compositions and/or formulations described herein include, by way of non-limiting example, mixtures of cetostearylic alcohol with sorbitan esterified with polyoxyethylenic fatty acids, polyoxyethylene fatty ethers, polyoxyethylene fatty esters, fatty acids, sulfated fatty acids, phosphated fatty acids, sulfosuccinates, amphoteric surfactants, non-ionic poloxamers, non-ionic meroxapols, petroleum derivatives, aliphatic amines, polysiloxane derivatives, sorbitan fatty acid esters, laureth-4, PEG-2 dilaurate, stearic acid, sodium lauryl sulfate, dioctyl sodium sulfosuccinate, cocoamphopropionate, poloxamer 188, meroxapol 258, triethanolamine, dimethicone, polysorbate 60, sorbitan monostearate, pharmaceutically acceptable salts thereof, and combinations thereof.

In some embodiments, non-ionic surfactants used in compositions and/or formulations described herein include, by way of non-limiting example, phospholipids, alkyl poly(ethylene oxide), poloxamers (e.g., poloxamer 188), polysorbates, sodium dioctyl sulfosuccinate, Brij™-30 (Laureth-4), Brij™-58 (Ceteth-20) and Brij™-78 (Steareth-20), Brij™-721 (Steareth-21), Crillet-1 (Polysorbate 20), Crillet-2 (Polysorbate 40), Crillet-3 (Polysorbate 60), Crillet 45 (Polysorbate 80), Myrj-52 (PEG-40 Stearate), Myrj-53 (PEG-50 Stearate), Pluronic™ F77 (Poloxamer 217), Pluronic™ F87 (Poloxamer 237), Pluronic™ F98 (Poloxamer 288), Pluronic™ L62 (Poloxamer 182), Pluronic™ L64 (Poloxamer 184), Pluronic™ F68 (Poloxamer 188), Pluronic™ L81 (Poloxamer 231), Pluronic™ L92 (Poloxamer 282), Pluronic™ L101 (Poloxamer 331), Pluronic™ P103 (Poloxamer 333), Pluracare™ F 108 NF (Poloxamer 338), and Pluracare™ F 127 NF (Poloxamer 407) and combinations thereof. Pluronic™ polymers are commercially purchasable from BASF, USA and Germany.

In certain embodiments, anionic surfactants used in compositions and/or formulations described herein include, by way of non-limiting example, sodium laurylsulphate, sodium dodecyl sulfate (SDS), ammonium lauryl sulfate, alkyl sulfate salts, alkyl benzene sulfonate, and combinations thereof.

In some embodiments, the cationic surfactants used in compositions and/or formulations described herein include, by way of non-limiting example, benzalkonium chloride, benzethonium chloride, cetyl trimethylammonium bromide, hexadecyl trimethyl ammonium bromide, other alkyltrimethylammonium salts, cetylpyridinium chloride, polyethoxylated tallow and combinations thereof.

In certain embodiments, the thickeners used in compositions and/or formulations described herein include, by way of non-limiting example, natural polysaccharides, semi-synthetic polymers, synthetic polymers, and combinations thereof. Natural polysaccharides include, by way of non-limiting example, acacia, agar, alginates, carrageenan, guar, arabic, tragacanth gum, pectins, dextran, gellan and xanthan gums. Semi-synthetic polymers include, by way of non-limiting example, cellulose esters, modified starches, modified celluloses, carboxymethylcellulose, methyl cellulose, ethyl cellulose, hydroxyethyl cellulose, hydroxypropyl cellulose and hydroxypropyl methylcellulose. Synthetic polymers include, by way of non-limiting example, polyoxyalkylenes, polyvinyl alcohol, polyacrylamide, polyacrylates, carboxypolymethylene (carbomer), polyvinylpyrrolidone (povidones), polyvinylacetate, polyethylene glycols and poloxamer. Other thickeners include, by way of nonlimiting example, polyoxyethyleneglycol isostearate, cetyl alcohol, Polyglycol 300 isostearate, propyleneglycol, collagen, gelatin, and fatty acids (e.g., lauric acid, myristic acid, palmitic acid, stearic acid, palmitoleic acid, linoleic acid, linolenic acid, oleic acid and the like).

In some embodiments, chelating agents used in the compositions and/or formulations described herein include, by way of non-limiting example, ethylenediaminetetraacetic acid (EDTA) or salts thereof, phosphates and combinations thereof.

In some embodiments, the concentration of the chelating agent or agents used in the rectal formulations described herein is a suitable concentration, e.g., about 0.1%, 0.15%, 0.2%, 0.25%, 0.3%, 0.4%, or 0.5% (w/v).

In some embodiments, preservatives used in compositions and/or formulations described herein include, by way of non-limiting example, parabens, ascorbyl palmitate, benzoic acid, butylated hydroxyanisole, butylated hydroxytoluene, chlorobutanol, ethylenediamine, ethylparaben, methylparaben, butyl paraben, propylparaben, monothioglycerol, phenol, phenylethyl alcohol, propylparaben, sodium benzoate, sodium propionate, sodium formaldehyde sulfoxylate, sodium metabisulfite, sorbic acid, sulfur dioxide, maleic acid, propyl gallate, benzalkonium chloride, benzethonium chloride, benzyl alcohol, chlorhexidine acetate, chlorhexidine gluconate, sorbic acid, potassium sorbitol, chlorbutanol, phenoxyethanol, cetylpyridinium chloride, phenylmercuric nitrate, thimerosol, and combnations thereof.

In certain embodiments, antioxidants used in compositions and/or formulations described herein include, by way of non-limiting example, ascorbic acid, ascorbyl palmitate, butylated hydroxyanisole, butylated hydroxytoluene, hypophosphorous acid, monothioglycerol, propyl gallate, sodium ascorbate, sodium sulfite, sodium bisulfite, sodium formaldehyde sulfoxylate, potassium metabisulphite, sodium metabisulfite, oxygen, quinones, t-butyl hydroquinone, erythorbic acid, olive (olea eurpaea) oil, pentasodium penetetate, pentetic acid, tocopheryl, tocopheryl acetate and combinations thereof.

In some embodiments, concentration of the antioxidant or antioxidants used in the rectal formulations described herein is sufficient to achieve a desired result, e.g., about 0.1%, 0.15%, 0.2%, 0.25%, 0.3%, 0.4%, or 0.5% (w/v).

The lubricating agents used in compositions and/or formulations described herein include, by way of non-limiting example, natural or synthetic fat or oil (e.g., a tris-fatty acid glycerate and the like). In some embodiments, lubricating agents include, by way of non-limiting example, glycerin (also called glycerine, glycerol, 1,2,3-propanetriol, and trihydroxypropane), polyethylene glycols (PEGs), polypropylene glycol, polyisobutene, polyethylene oxide, behenic acid, behenyl alcohol, sorbitol, mannitol, lactose, polydimethylsiloxane and combinations thereof.

In certain embodiments, mucoadhesive and/or bioadhesive polymers are used in the compositions and/or formulations described herein as agents for inhibiting absorption of the enteroendocrine peptide secretion enhancing agent across the rectal or colonic mucosa. Bioadhesive or mucoadhesive polymers include, by way of non-limiting example, hydroxypropyl cellulose, polyethylene oxide homopolymers, polyvinyl ether-maleic acid copolymers, methyl cellulose, ethyl cellulose, propyl cellulose, hydroxyethyl cellulose, hydroxypropyl cellulose, hydroxypropylmethyl cellulose, carboxymethylcellulose, polycarbophil, polyvinylpyrrolidone, carbopol, polyurethanes, polyethylene oxide-polypropyline oxide copolymers, sodium carboxymethyl cellulose, polyethylene, polypropylene, lectins, xanthan gum, alginates, sodium alginate, polyacrylic acid, chitosan, hyaluronic acid and ester derivatives thereof, vinyl acetate homopolymer, calcium polycarbophil, gelatin, natural gums, karaya, tragacanth, algin, chitosan, starches, pectins, and combinations thereof.

In some embodiments, bufferspH adjusting agents used in compositions and/or formulations described herein include, by way of non-limiting example, phosphoric acid, monobasic sodium or potassium phosphate, triethanolamine (TRIS), BICINE, HEPES, Trizma, glycine, histidine, arginine, lysine, asparagine, aspartic acid, glutamine, glutamic acid, carbonate, bicarbonate, potassium metaphosphate, potassium phosphate, monobasic sodium acetate, acetic acid, acetate, citric acid, sodium citrate anhydrous, sodium citrate dihydrate and combinations thereof. In certain embodiments, an acid or a base is added to adjust the pH. Suitable acids or bases include, by way of non-limiting example, HCL, NaOH and KOH.

In certain embodiments, concentration of the buffering agent or agents used in the rectal formulations described herein is sufficient to achieve or maintain a physiologically desirable pH, e.g., about 0.1%, 0.2%, 0.3%, 0.4%, 0.5%, 0.6%, 0.8%, 0.9%, or 1.0% (w/w).

The tonicity modifiers used in compositions and/or formulations described herein include, by way of non-limiting example, sodium chloride, potassium chloride, sodium phosphate, mannitol, sorbitol or glucose. Formulations

In certain aspects, the composition or formulation containing one or more compounds described herein is orally administered for local delivery of an ASBTI, or a compound described herein to the gastrointestinal site of action. Unit dosage forms of such compositions include a pill, tablet or capsules formulated for enteric delivery. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry starch, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starches, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

In some embodiments, ASBTIs, or other compounds described herein are orally administered in association with a carrier suitable for delivery to the distal gastrointestinal tract (e.g., jejunum, ileum, colon, and/or rectum).

In certain embodiments, a composition described herein comprises an ASBTI, or other compounds described herein in association with a matrix (e.g., a matrix comprising hypermellose) that allows for controlled release of an active agent in the distal part of the ileum and/or the colon. In some embodiments, a composition comprises a polymer that is pH sensitive (e.g., a MMX™ matrix from Cosmo Pharmaceuticals) and allows for controlled release of an active agent in the distal part of the ileum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a composition suitable for controlled release in the distal ileum comprises microparticulate active agent (e.g., micronized active agent). In some embodiments, a non-enzymatically degrading poly(dl-lactide-co-glycolide) (PLGA) core is suitable for delivery of an enteroendocrine peptide secretion enhancing agent (e.g., bile acid) to the distal ileum. In some embodiments, a dosage form comprising an enteroendocrine peptide secretion enhancing agent (e.g., bile acid) is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the distal ileum and/or the colon. In some embodiments, bacterially activated systems are suitable for targeted delivery to the distal part of the ileum. Examples of micro-flora activated systems include dosage forms comprising pectin, galactomannan, and/or Azo hydrogels and/or glycoside conjugates (e.g., conjugates of D-galactoside, (3-D-xylopyranoside or the like) of the active agent. Examples of gastrointestinal micro-flora enzymes include bacterial glycosidases such as, for example, D-galactosidase, 13-D-glucosidase, α-L-arabinofuranosidase, 13-D-xylopyranosidase or the like.

The pharmaceutical composition described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In further embodiments, a tablet or capsule comprising an ASBTI or other compounds described herein is film-coated for delivery to targeted sites within the gastrointestinal tract. Examples of enteric film coats include and are not limited to hydroxypropylmethylcellulose, polyvinyl pyrrolidone, hydroxypropyl cellulose, polyethylene glycol 3350, 4500, 8000, methyl cellulose, pseudo ethylcellulose, amylopectin and the like.

### Bile Acid Sequestrant

In certain embodiments, an oral formulation for use in any method described herein is, e.g., an ASBTI in association with a labile bile acid sequestrant. A labile bile acid sequestrant is a bile acid sequestrant with a labile affinity for bile acids. In certain embodiments, a bile acid sequestrant described herein is an agent that sequesters (e.g., absorbs or is charged with) bile acid, and/or the salts thereof.

In specific embodiments, the labile bile acid sequestrant is an agent that sequesters (e.g., absorbs or is charged with) bile acid, and/or the salts thereof, and releases at least a portion of the absorbed or charged bile acid, and/or salts thereof in the distal gastrointestinal tract (e.g., the colon, ascending colon, sigmoid colon, distal colon, rectum, or any combination thereof). In certain embodiments, the labile bile acid sequestrant is an enzyme dependent bile acid sequestrant. In specific embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, 13-D-xylopyranoside or the like) of the active agent. Examples of gastrointestinal micro-flora enzymes include bacterial glycosidases such as, for example, D-galactosidase, 13-D-glucosidase, α-L-arabinofuranosidase, 13-D-xylopyranosidase or the like. In some embodiments, the labile bile acid sequestrant is a time dependent bile acid sequestrant (i.e., the bile acid sequesters the bile acid and/or salts thereof and after a time releases at least a portion of the bile acid and/or salts thereof). In some embodiments, a time dependent bile acid sequestrant is an agent that degrades in an aqueous environment over time. In certain embodiments, a labile bile acid sequestrant described herein is a bile acid sequestrant that has a low affinity for bile acid and/or salts thereof, thereby allowing the bile acid sequestrant to continue to sequester bile acid and/or salts thereof in an environ where the bile acidssalts and/or salts thereof are present in high concentration and release them in an environ wherein bile acidssalts and/or salts thereof are present in a lower relative concentration. In some embodiments, the labile bile acid sequestrant has a high affinity for a primary bile acid and a low affinity for a secondary bile acid, allowing the bile acid sequestrant to sequester a primary bile acid or salt thereof and subsequently release a secondary bile acid or salt thereof as the primary bile acid or salt thereof is converted (e.g., metabolized) to the secondary bile acid or salt thereof. In some embodiments, the labile bile acid sequestrant is a pH dependent bile acid sequestrant. In some embodiments, the pH dependent bile acid sequestrant has a high affinity for bile acid at a pH of 6 or below and a low affinity for bile acid at a pH above 6. In certain embodiments, the pH dependent bile acid sequestrant degrades at a pH above 6.

In some embodiments, labile bile acid sequestrants described herein include any compound, e.g., a macro-structured compound, that can sequester bile acidssalts and/or salts thereof through any suitable mechanism. For example, in certain embodiments, bile acid sequestrants sequester bile acidssalts and/or salts thereof through ionic interactions, polar interactions, static interactions, hydrophobic interactions, lipophilic interactions, hydrophilic interactions, steric interactions, or the like. In certain embodiments, macrostructured compounds sequester bile acidssalts and/or sequestrants by trapping the bile acidssalts and/or salts thereof in pockets of the macrostructured compounds and, optionally, other interactions, such as those described herein. In some embodiments, bile acid sequestrants (e.g., labile bile acid sequestrants) include, by way of non-limiting example, lignin, modified lignin, polymers, polycationic polymers and copolymers, polymers and/or copolymers comprising anyone one or more of N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof, or any combination thereof.

Covalent Linkage of the Drug with a Carrier

In some embodiments, strategies used for colon targeted delivery include, by way of non-limiting example, covalent linkage of the ASBTI or other compounds described herein to a carrier, coating the dosage form with a pH-sensitive polymer for delivery upon reaching the pH environment of the colon, using redox sensitive polymers, using a time released formulation, utilizing coatings that are specifically degraded by colonic bacteria, using bioadhesive system and using osmotically controlled drug delivery systems.

In certain embodiments of such oral administration of a composition containing an ASBTI or other compounds described herein involves covalent linking to a carrier wherein upon oral administration the linked moiety remains intact in the stomach and small intestine. Upon entering the colon the covalent linkage is broken by the change in pH, enzymes, and/or degradation by intestinal microflora. In certain embodiments, the covalent linkage between the ASBTI and the carrier includes, by way of non-limiting example, azo linkage, glycoside conjugates, glucuronide conjugates, cyclodextrin conjugates, dextran conjugates, and amino-acid conjugates (high hydrophilicity and long chain length of the carrier amino acid).

Coating with Polymers: pH-Sensitive Polymers

In some embodiments, the oral dosage forms described herein are coated with an enteric coating to facilitate the delivery of an ASBTI or other compounds described herein to the colon and/or rectum. In certain embodiments, an enteric coating is one that remains intact in the low pH environment of the stomach, but readily dissolved when the optimum dissolution pH of the particular coating is reached which depends upon the chemical composition of the enteric coating. The thickness of the coating will depend upon the solubility characteristics of the coating material. In certain embodiments, the coating thicknesses used in such formulations described herein range from about 25 μm to about 200 μm.

In certain embodiments, the compositions or formulations described herein are coated such that an ASBTI or other compounds described herein of the composition or formulation is delivered to the colon and/or rectum without absorbing at the upper part of the intestine. In a specific embodiment, specific delivery to the colon and/or rectum is achieved by coating of the dosage form with polymers that degrade only in the pH environment of the colon. In alternative embodiments, the composition is coated with an enteric coat that dissolves in the pH of the intestines and an outer layer matrix that slowly erodes in the intestine. In some of such embodiments, the matrix slowly erodes until only a core composition comprising an enteroendocrine peptide secretion enhancing agent (and, in some embodiments, an absorption inhibitor of the agent) is left and the core is delivered to the colon and/or rectum.

In certain embodiments, pH-dependent systems exploit the progressively increasing pH along the human gastrointestinal tract (GIT) from the stomach (pH 1-2 which increases to 4 during digestion), small intestine (pH 6-7) at the site of digestion and it to 7-8 in the distal ileum. In certain embodiments, dosage forms for oral administration of the compositions described herein are coated with pH-sensitive polymer(s) to provide delayed release and protect the enteroendocrine peptide secretion enhancing agents from gastric fluid. In certain embodiments, such polymers are be able to withstand the lower pH values of the stomach and of the proximal part of the small intestine, but disintegrate at the neutral or slightly alkaline pH of the terminal ileum and/or ileocecal junction. Thus, in certain embodiments, provided herein is an oral dosage form comprising a coating, the coating comprising a pH-sensitive polymer. In some embodiments, the polymers used for colon and/or rectum targeting include, by way of non-limiting example, methacrylic acid copolymers, methacrylic acid and methyl methacrylate copolymers, Eudragit L100, Eudragit S100, Eudragit L-30D, Eudragit FS-30D, Eudragit L100-55, polyvinylacetate phthalate, hyrdoxypropyl ethyl cellulose phthalate, hyrdoxypropyl methyl cellulose phthalate 50, hyrdoxypropyl methyl cellulose phthalate 55, cellulose acetate trimelliate, cellulose acetate phthalate and combinations thereof.

In certain embodiments, oral dosage forms suitable for delivery to the colon and/or rectum comprise a coating that has a biodegradable and/or bacteria degradable polymer or polymers that are degraded by the microflora (bacteria) in the colon. In such biodegradable systems suitable polymers include, by way of non-limiting example, azo polymers, linear-type-segmented polyurethanes containing azo groups, polygalactomannans, pectin, glutaraldehyde crosslinked dextran, polysaccharides, amylose, guar gum, pectin, chitosan, inulin, cyclodextrins, chondroitin sulphate, dextrans, locust bean gum, chondroitin sulphate, chitosan, poly (-caprolactone), polylactic acid and poly(lactic-co-glycolic acid).

In certain embodiments of such oral administration of compositions containing one or more ASBTIs or other compounds described herein, the compositions are delivered to the colon without absorbing at the upper part of the intestine by coating of the dosage forms with redox sensitive polymers that are degraded by the microflora (bacteria) in the colon. In such biodegradable systems such polymers include, by way of non-limiting example, redox-sensitive polymers containing an azo and/or a disulfide linkage in the backbone.

In some embodiments, compositions formulated for delivery to the colon and/or rectum are formulated for time-release. In some embodiments, time release formulations resist the acidic environment of the stomach, thereby delaying the release of the enteroendocrine peptide secretion enhancing agents until the dosage form enters the colon and/or rectum.

In certain embodiments the time released formulations described herein comprise a capsule (comprising an enteroendocrine peptide secretion enhancing agent and an optional absorption inhibitor) with hydrogel plug. In certain embodiments, the capsule and hydrogel plug are covered by a water-soluble cap and the whole unit is coated with an enteric polymer. When the capsule enters the small intestine the enteric coating dissolves and the hydrogels plug swells and dislodges from the capsule after a period of time and the composition is released from the capsule. The amount of hydrogel is used to adjust the period of time to the release the contents.

In some embodiments, provided herein is an oral dosage form comprising a multi-layered coat, wherein the coat comprises different layers of polymers having different pH-sensitivities. As the coated dosage form moves along GIT the different layers dissolve depending on the pH encountered. Polymers used in such formulations include, by way of non-limiting example, polymethacrylates with appropriate pH dissolution characteristics, Eudragit® RL and Eudragit®RS (inner layer), and Eudragit® FS (outer layer). In other embodiments the dosage form is an enteric coated tablets having an outer shell of hydroxypropylcellulose or hydroxypropylmethylcellulose acetate succinate (HPMCAS).

In some embodiments, provided herein is an oral dosage form that comprises coat with cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and combinations thereof.

Combination Therapy with Fat Soluble Vitamins

In some embodiments, the methods provided herein further comprise administering one or more vitamins. In some embodiments, the vitamin is vitamin A, B1, B2, B3, B5, B6, B7, B9, B12, C, D, E, K, folic acid, pantothenic acid, niacin, riboflavin, thiamine, retinol, beta carotene, pyridoxine, ascorbic acid, cholecalciferol, cyanocobalamin, tocopherols, phylloquinone, menaquinone.

In some embodiments, the vitamin is a fat soluble vitamin such as vitamin A, D, E, K, retinol, beta carotene, cholecalciferol, tocopherols, phylloquinone. In a preferred embodiment, the fat soluble vitamin is tocopherol polyethylene glycol succinate (TPGS).

Combination Therapy with Partial External Biliary Diversion (PEBD)

In some embodiments, the methods provided herein further comprise using partial external biliary diversion as a treatment for patients who have not yet developed cirrhosis. This treatment helps reduce the circulation of bile acidssalts in the liver in order to reduce complications and prevent the need for early transplantation in many patients.

This surgical technique involves isolating a segment of intestine 10 cm long for use as a biliary conduit (a channel for the passage of bile) from the rest of the intestine. One end of the conduit is attached to the gallbladder and the other end is brought out to the skin to form a stoma (a surgically constructed opening to permit the passage of waste). Partial external biliary diversion may be used for patients who are unresponsive to all medical therapy, especially older, larger patients. This procedure may not be of help to young patients such as infants. Partial external biliary diversion may decrease the intensity of the itching and abnormally low levels of cholesterol in the blood.

Combination Therapy with ASBTI and Ursodiol

In some embodiments, an ASBTI is administered in combination with ursodiol or ursodeoxycholic acid, chenodeoxycholic acid, cholic acid, taurocholic acid, ursocholic acid, glycocholic acid, glycodeoxycholic acid, taurodeoxycholic acid, taurocholate, glycochenodeoxycholic acid, tauroursodeoxycholic acid. In some instances an increase in the concentration of bile acidssalts in the distal intestine induces intestinal regeneration, attenuating intestinal injury, reducing bacterial translocation, inhibiting the release of free radical oxygen, inhibiting production of proinflammatory cytokines, or any combination thereof or any combination thereof.

An ASBTI and a second active ingredient are used such that the combination is present in a therapeutically effective amount. That therapeutically effective amount arises from the use of a combination of an ASBTI and the other active ingredient (e.g., ursodiol) wherein each is used in a therapeutically effective amount, or by virtue of additive or synergistic effects arising from the combined use, each can also be used in a subclinical therapeutically effective amount, i.e., an amount that, if used alone, provides for reduced effectiveness for the therapeutic purposes noted herein, provided that the combined use is therapeutically effective. In some embodiments, the use of a combination of an ASBTI and any other active ingredient as described herein encompasses combinations where the ASBTI or the other active ingredient is present in a therapeutically effective amount, and the other is present in a subclinical therapeutically effective amount, provided that the combined use is therapeutically effective owing to their additive or synergistic effects. As used herein, the term “additive effect” describes the combined effect of two (or more) pharmaceutically active agents that is equal to the sum of the effect of each agent given alone. A synergistic effect is one in which the combined effect of two (or more) pharmaceutically active agents is greater than the sum of the effect of each agent given alone. Any suitable combination of an ASBIT with one or more of the aforementioned other active ingredients and optionally with one or more other pharmacologically active substances is contemplated as being within the scope of the methods described herein.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy described herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, provided herein are combination therapies. In certain embodiments, the compositions described herein comprise an additional therapeutic agent. In some embodiments, the methods described herein comprise administration of a second dosage form comprising an additional therapeutic agent. In certain embodiments, combination therapies the compositions described herein are administered as part of a regimen. Therefore, additional therapeutic agents and/or additional pharmaceutical dosage form can be applied to a patient either directly or indirectly, and concomitantly or sequentially, with the compositions and formulations described herein.

### Kits

In another aspect, provided herein are kits containing a device for rectal administration pre-filled a pharmaceutical composition described herein. In certain embodiments, kits contain a device for oral administration and a pharmaceutical composition as described herein. In certain embodiments the kits includes prefilled sachet or bottle for oral administration, while in other embodiments the kits include prefilled bags for administration of rectal gels. In certain embodiments the kits includes prefilled syringes for administration of oral enemas, while in other embodiments the kits include prefilled syringes for administration of rectal gels. In certain embodiments the kits includes prefilled pressurized cans for administration of rectal foams.

### Pharmaceutical Compositions

Provided herein, in certain embodiments, is a pharmaceutical composition comprising a therapeutically effective amount of any compound described herein. In certain instances, the pharmaceutical composition comprises an ASBT inhibitor (e.g., any ASBTI described herein). In certain instances, the pharmaceutical composition consists essentially of an ASBT inhibitor (e.g., any ASBTI described herein).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

A pharmaceutical composition, as used herein, refers to a mixture of a compound described herein, such as, for example, a compound of Formula I-VI, with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

In certain embodiments, a pharmaceutical compositions described herein includes one or more compound described herein as an active ingredient in free-acid or free-base form, or in a pharmaceutically acceptable salt form. In some embodiments, the compounds described herein are utilized as an N-oxide or in a crystalline or amorphous form (i.e., a polymorph). In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be described herein.

A “carrier” includes, in some embodiments, a pharmaceutically acceptable excipient and is selected on the basis of compatibility with compounds described herein, such as, compounds of any of Formula I-VI, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of Formula I-VI. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

An ASBT inhibitor (e.g., a compound of Formula I-VI) is used in the preparation of medicaments for the prophylactic and/or therapeutic treatment of Barrett's esophagus or GERD. A method for treating any of the diseases or conditions described herein in an individual in need of such treatment, involves administration of pharmaceutical compositions containing at least one ASBT inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

### Screening Process

Provided in certain embodiments herein are processes and kits for identifying compounds suitable for treating Barrett's esophagus or GERD. In certain embodiments, provided herein are assays for identifying compounds that selectively inhibits the ASBT by:
a. providing cells that are a model of intestinal cells;
b. contacting the cells with a compound (e.g., a compound as described herein);
c. detecting or measuring the effect of the compound on the inhibition of ASBT activity.

In certain embodiments, provided herein are assays for identifying compounds that are non-systemic compounds by
a. providing cells that are a model of intestinal permeability (e.g., Caco-2 cells);
b. culturing the cells as a monolayer on semi-permeable plastic supports that are fitted into the wells of multi-well culture plates;
c. contacting the apical or basolateral surface of the cells with a compound (e.g., a compound as described herein) and incubating for a suitable length of time;
d. detecting or measuring the concentration of the compound on both sides of the monolayer by liquid-chromatography-mass spectrometry (LC-MS) and computing intestinal permeability of the compound.

In certain embodiments, non-systemic compounds are identified by suitable parallel artificial membrane permeability assays (PAMPA).

In certain embodiments, non-systemic compounds are identified by use of isolated vascular-perfused gut preparations.

In certain embodiments, provided herein are assays for identifying compounds that inhibit recycling of bile acid salts by
a. providing cells that are a model of intestinal cells with apical bile acid transporters (e.g., BHK cells, CHO cells);
b. incubating the cells with a compound (e.g., a compound as described herein) and/or a radiolabeled bile acid (e.g., ¹⁴C taurocholate) for a suitable length of time;
c. washing the cells with a suitable buffer (e.g. phosphate buffered saline);
d. detecting or measuring the residual concentration of the radiolabeled bile acid in the cells.

### EXAMPLES

### Example 1

### Synthesis of 1-phenethyl-1-((1,4-diazabicyclo[2.2.2]octanyl)pentyl)imidodicarbonimidic diamide, iodide salt

[chemical structure]

### Step 1: Synthesis of 5-(1,4-diazabicyclo[2.2.2]octanyl)-1-iodo pentane, iodide salt

[chemical structure]

1,4-diazabicyclo[2.2.2]octane is suspended in THF. Diiodopentane is added dropwise and the mixture is refluxed overnight. The reaction mixture is filtered.

### Step 2: Synthesis of N-phenethyl-5-(1,4-diazabicyclo[2.2.2]octanyl)-1-iodo pentane, iodide salt

[chemical structure]

5-(1,4-diazabicyclo[2.2.2]octanyl)-1-iodo pentane, iodide salt is suspended in acetonitrile. Phenethylamine is added dropwise and the mixture is refluxed overnight. The reaction mixture is filtered.

### Step 3: Synthesis of 1-phenethyl-1-((1,4-diazabicyclo[2.2.2]octanyl)pentyl)imidodicarbonimidic diamide, iodide salt

N-phenethyl-5-(1,4-diazabicyclo[2.2.2]octanyl)-1-iodo pentane, iodide salt is heated with dicyanodiamide in n-butanol for 4 h. The reaction mixture is concentrated under reduced pressure.

The compounds below are prepared using methods as described herein, and using appropriate starting materials.

| Compound | |
|---|---|
| No. | Structure |
| 1 | [chemical structure] |
| 2 | [chemical structure] |
| 3 | [chemical structure] |
| 4 | [chemical structure] |
| 5 | [chemical structure] |
| 6 | [chemical structure] |
| 7 | [chemical structure] |
| 8 | [chemical structure] |
| 9 | [chemical structure] |
| 10 | [chemical structure] |
| 11 | [chemical structure] |

### Example 2

### In Vitro Assay for Inhibition of ASBT-Mediated Bile Acid Uptake

Baby hamster kidney (BHK) cells are transfected with cDNA of human ASBT. The cells are seeded in 96-well tissue culture plates at 60,000 cellswell. Assays are run within 24 hours of seeding.

On the day of the assay the cell monolayer is washed with 100 mL of assay buffer. The test compound is added to each well along with 6 mM [¹⁴C] taurocholate in assay buffer (final concentration of 3 mM [¹⁴C] taurocholate in each well). The cell cultures are incubated for 2 h at 37° C. The wells are washed with PBS. Scintillation counting fluid is added to each well, the cells are shaken for 30 minutes prior to measuring amount of radioactivity in each well. A test compound that has significant ASBT inhibitory activity provides an assay wherein low levels of radioactivity are observed in the cells.

### Example 3

### In Vitro Assay for Secretion of GLP-2

Human NCI-H716 cells are used as a model for L-cells. Two days before each assay experiment, cells are seeded in 12-well culture plates coated with Matrigel® to induce cell adhesion. On the day of the assay, cells are washed with buffer. The cells are incubated for 2 hours with medium alone, or with test compound. The extracellular medium is assayed for the presence of GLP-2. Peptides in the medium are collected by reverse phase adsorption and the extracts are stored until assay. The presence of GLP-2 is assayed using ELISA. The detection of increased levels of GLP-2 in a well containing a test compound identifies the test compound as a compound that can enhance GLP-2 secretions from L-cells.

### Example 4

### In Vivo Bioavailability Assay

The test compounds are solubilized in saline solutions. Sprague Dawley rats are dosed at 2-10 mg/kg body weight by iv and oral dosing. Peripheral blood samples are taken from the femoral artery at selected time periods up to 8 hours. Plasma concentrations of the compounds are determined by quantitative HPLC and/or mass spectrometry. Clearance and AUC values are determined for the compounds.

For oral dosing, bioavailability is calculated by also drawing plasma samples from the portal vein. Cannulae are inserted in the femoral artery and the hepatic portal vein to obtain estimates of total absorption of drug without first-pass clearance in the liver. The fraction absorbed (F) is calculated by

F=AUCₚₒ/AUCᵢᵥ

### Example 5

### Assay to Determine Ileal Intraenterocyte and Luminal Bile Acid Levels

Ileal luminal bile acid levels in SD rats are determined by flushing a 3-cm section of distal ileum with sterile, cold PBS. After flushing with additional PBS, the same section of ileum is weighed and then homogenized in fresh PBS for determination of interenterocyte bile acid levels. A LCMSMS system is used to evaluate cholic acid, DCA, LCA, chenodeoxycholic acid, and ursodeoxycholic acid levels.

### Example 6

### Animal to Determine Effect of Therapy on Barrett's Esophagus or GERD

Mdr2 knock out mouse model or Barrett's esophagus or GERD induced rats (by carbon tetrachloridephenobarbital) is used to test compositions described herein. The animals are orally administered a composition comprising an ASBTI.

Barrett's esophagus or GERD is quantitated by total bile acid and bilirubin in serum versus that in control micerats administered with placebo. Serum bile acidssalts are determined by ELISA with specific antibodies for cholic and CCDCA. Serum bilirubin levels are determined by automated routine assays. Alternatively, livers of the mice can be harvested and pathology of the hepatocellular damage can be measured.

### Example 7

Investigation of orally delivered LUM001 and 1-[4-[4-[(4R,5R)-3,3-dibutyl-7-(dimethylamino)-2,3,4,5-tetrahydro-4-hydroxy-1,1-di oxido-1-benzothiepin-5-yl]phenoxy]butyl] 4-aza-1-azoniabicyclo[2.2.2] octane methane sulfonate (Compound 100B) on plasma GLP-2 levels in normal rats

12-week-old male HSD rats are fasted for 16 h and given oral dose of 0, 3, 30, 100 mg/kg of the ASBTIs LUM001 or 1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl] 4-aza-1-azoniabicyclo[2.2.2] octane methane sulfonate (Synthesized by Nanosyn Inc., CA, USA) in a mixture of valine-pyrrolidine in water (n=5 per group). Blood samples in volume of 0.6 ml for each time point are taken from the caudal vein with a heparinized capillary tube 0, 1, 3 and 5 h after the administration of compounds and plasma GLP-2 level are determined. Aprotinin and 10 μl of DPP-IV inhibitor per ml of blood are used for blood sample preservation during 10 min centrifugation and for storage at −70° C. or below. GLP-2 (Active pM) is tested by any commercially available ELISA kits.

### Example 8

### Tablet Formulation

10 kg of a compound of Formula I-VI is first screened through a suitable screen (e.g. 500 micron). 25 kg Lactose monohydrate, 8 kg hydroxypropylmethyl cellulose, the screened compound of Formula I-VI and 5 kg calcium hydrogen phosphate (anhydrous) are then added to a suitable blender (e.g. a tumble mixer) and blended. The blend is screened through a suitable screen (e.g. 500 micron) and reblended. About 50% of the lubricant (2.5 kg, magnesium stearate) is screened, added to the blend and blended briefly. The remaining lubricant (2 kg, magnesium stearate) is screened, added to the blend and blended briefly. The granules are screened (e.g. 200 micron) to obtain granulation particles of the desired size. In some embodiments, the granules are optionally coated with a drug release controlling polymer such as polyvinylpyrrolidone, hydroxypropylcellulose, hydroxypropylmethyl cellulose, methyl cellulose, or a methacrylic acid copolymer, to provide an extended release formulation. The granules are filled in gelatin capsules.

### Example 9

### Pediatric Formulation

Disintegrating Tablet Formulation

The following example describes a large scale preparation (100 kg) of an ASBTI compound of Formula I-VI (e.g., LUM-001 or LUM-002).

| | | |
|---|---|---|
| Active ingredient (LUM-001) | 2.5 | kg |
| Lactose monohydrate NF | 47.5 | kg |
| Pregelatinized starch NF | 18 | kg |
| microcrystalline cellulose NF | 17 | kg |
| croscarmellose sodium NF | 6.5 | kg |
| povidone K29/32 USP | 8.5 | kg |
| | 100 | kg |

Pass ASBTI (2.5 kg), lactose monohydrate NF (47.5 kg), pregelatinized starch NF (18 kg), microcrystalline cellulose NF (17 kg), croscarmellose sodium NF (6.5 kg) and povidone K2932 USP (8.5 kg) through a #10 mesh screen. Add the screened material to a 600 Collette mixer. Mix for 6 minutes at low speed, without chopper. Add the direct blend mixture from the previous step to a 20-cubic foot V-shell PK blender (Model C266200). Pass magnesium stearate NF (0.5 to 1 kg) through a 10 mesh screen into a properly prepared container. Add approximately half of the magnesium stearate to each side of the PK blender and blend for 5 minutes. Add the blended mixture from the previous step to Kikusui tablet press for compression into tablets. The compression equipment can be outfitted to make tooling for 50 mg tablet, 75 mg tablet and 100 mg tablet.

### Example 10

### Chewable Tablet

A 40% (w/w) solution of the Eudragit E100 in ethanol was added with mixing to the active ingredient and blended until granules were formed. The resulting granules were dried and then sieved through a 16 mesh screen.

| | | |
|---|---|---|
| Active ingredient | 4.0 | mg |
| Eudragit E100 | 0.6 | mg |
| Sorbitol: Direct Compression Grade | 18.8 | mg |
| Lactose: Direct Compression Grade | 15.6 | mg |
| Croscarmellose Sodium Type A | 1.2 | mg |
| Aspartame | 0.3 | mg |
| Aniseed flavoring | 0.6 | mg |
| Butterscotch flavoring | 0.6 | mg |
| Magnesium Stearate | 0.6 | mg |
| Microcrystalline Cellulose | 4.7 | mg |
| (Avicel PH102) | | |
| | 47 | mg |

The active ingredient granules and extragranular excipients were put into a cone blender and mixed thoroughly. The resulting mix was discharged from the blender and compressed on a suitable rotary tablet press fitted with the appropriate punches.

### Example 11

### Animal Study

Animal Preparation.

Male Zucker diabetic fatty rats (ZDF/GmiCrl-fa/fa) were purchased from Charles River (Raleigh, N.C.) and housed under controlled conditions (12:12 light-dark cycle, 24° C. and 50% relative humidity) with free access to rodent food (Purina 5008, Harlan Teklad, Indianapolis, Ind.). All rats arrived at seven weeks of age (±3 days). After a one-week acclimation period, rats were anesthetized with isoflurane (Abbott Laboratories, IL) and tail-vein blood samples were collected at 9 am without fasting. Blood glucose levels were measured using a glucometer (Bayer, Leverkusen, Germany). In order to ensure balanced treatment groups, ZDF rats were assigned to six treatment groups based upon baseline glucose: vehicle (0.5% HPMC, 0.1% Tween80) and five doses of 264W94 (0.001, 0.01, 0.1, 1, 10 mg/kg). All treatments were given via oral gavage twice a day and animals were followed for two weeks with blood samples collected from tail vein at the end of each week at 9 am without fasting. Fecal samples were collected for 24 hours during the second week of treatment.

Measurement of Clinical Chemistry Parameters.

Non-esterified fatty acids (NEFA), bile acids, and bile acids in fecal extraction were measured using the Olympus AU640 clinical chemistry analyzer (Beckman Coulter, Irving, Tex.).

Changes in Fecal Bile Acid Excretion and Plasma Bile Acid Concentrations.

Oral administration of 264W94 dose-dependently increased bile acids in the feces. Fecal bile acid concentrations were elevated up to 6.5 fold with an ED₅₀ of 0.17 mg/kg, when compared to vehicle treated rats. Fecal NEFA also slightly increased in 264W94 treated rats. In contrast, plasma bile acid concentrations were decreased dose-dependently in 264W94 treated rats. See FIG. 1.

Plasma Bile Acid Levels of ZDF Rats after Administration of Ascending Doses of SC-435 and LUM002.

Male ZDF rats (n=4) were administered vehicle, SC-435 (1, 10 or 30 mg/kg) or LUM002 (0.3, 1, 3, 10 or 30 mg/kg) by oral gavage twice a day for 2 weeks. Plasma bile acid levels were determined at the end of the second week. Plasma bile acid levels were decreased for all doses of SC-435 and LUM002. Data are expressed as mean values±SEM. See FIG. 2.

### Example 12

### Animal Study on the Duration of Action and Time to Onset of ASBTI Activity of a Single Oral Dose of LUM001 on Postprandial Total Serum Bile Acids in Beagle Dogs

Test Compound:

LUM001-Form I

Dosage Preparation and Administration:

LUM001 was dissolved in water at concentrations that required the administration of 0.2 ml/kg of solution. Solutions were placed into gelatin capsules, Torpac Inc., size 13 Batch 594, East Hanover N.J., and administered orally.

Dogs:

Male beagle dogs were obtained from Covance Research Products, Cumberland Va. or Marshall Farms USA, Inc., North Rose N.Y. A total of 20 dogs, 1 to 5 years old, 6.8 to 15.6 kg body weight, were used in these experiments. The dogs were conditioned to a 12 hour light/dark cycle and maintained on a feeding restriction of 1 hour per day access to food (Richman Standard Certified Canine Diet #5007, PMI Nutrition, Inc., St. Louis Mo.) from 7 to 8 AM. They were trained to eat a special meal promptly within 20 minutes when presented (1 can. 397 g, Evanger's 100% Beef for Dogs, Evanger's Dog and Cat Food Co., Inc., Wheeling Ill., mixed with 50 g of sharp cheddar cheese.).

Serum Total Bile Acid (SBA) Measurement:

SBA was measured by an enzymatic assay. SBA values are expressed as μg of total bile acids/ml of serum.

Control Experiments to Estimate the Rise and Duration of Elevation in Systemic Serum Bile Acid:

Previous work demonstrated that SBA of beagle dogs rises to a peak level one hour after feeding the meal described herein, and remains at a plateau for 4 hours and then declines. To estimate the details of this plateau, 6 dogs were given a test meal and blood samples for SBA measurement were collected at −30, 0, 30, 60, 65, 70, 80, 90, 120, 180, 240, 360, 480, 720, 1410 and 1440 minutes from the time of feeding. Any remaining food was removed 20 min after it was first presented to the dogs. To establish a method for extending the elevated plateau of SBA, 6 dogs were given the meal at 0 hr and an additional ½ size meal again 4 hr after their first meal. Blood samples were taken at 0, 1, 2, 3, 4, 4.5, 6, 7 and 8 hr. The curves for SBA level vs time obtained in these experiments were used as references for determining blood sampling times in experiments with LUM001. Wherever possible, experimental design permitting, in experiments with test compound, each dog served as its own simultaneous control, and the mean 1 hr SBA value served as the reference to which all other mean values were compared.

Experiments to Measure Time to Onset of Activity of LUM001:

LUM001 was administered at 0, 0.01, 0.05, 0.2 and 1 mg/kg, p.o. to dogs, n=6, 1 hr after feeding the standard experimental meal. Blood samples for SBA measurement were taken at −30, 0, 30, 60, 65, 70, 80, 90, 120 and 180 minutes from the time of feeding. Each dog served as its own control, and mean SBA levels were compared to the mean SBA level at 60 minutes.

TABLE 1
Onset of Activity of LUM001 on Dog Serum Bile Acids

| SD-5613 | Water, | | 0.01 mg/kg, | | 0.05 mg/kg, | | 0.2 mg/kg, | | 1 mg/kg, | |
|---|---|---|---|---|---|---|---|---|---|---|
| Time | n = 6 | | n = 6 | | n = 6 | | n = 6 | | n = 6 | |
| (min) | Mean | sem | Mean | sem | Mean | sem | Mean | sem | Mean | sem |
| −30 | 2.2 | 0.3 | 1.5 | 0.1 | 1.4 | 0.1 | 2.4 | 0.5 | 2.1 | 0.2 |
| 0 | 2.0 | 0.3 | 1.4 | 0.1 | 2.1 | 0.6 | 1.9 | 0.2 | 2.8 | 0.4 |
| 30 | 6.9 | 2.1 | 5.8 | 2.5 | 6.8 | 2.3 | 9.1 | 2.1 | 7.6 | 1.8 |
| 60 | 17.8 | 3.2 | 14.6 | 2.8 | 10.4 | 1.2 | 19.1 | 2.7 | 13.8 | 1.4 |
| 65 | 16.6 | 3.6 | 13.9 | 2.4 | 12.2 | 1.7 | 14.9 | 1.7 | 13.5 | 1.4 |
| 70 | 16.2 | 1.9 | 14.1 | 2.2 | 12.0 | 1.6 | 16.7 | 2.3 | 15.4 | 1.8 |
| 80 | 16.1 | 2.3 | 12.8 | 1.8 | 10.0 | 1.3 | 14.3 | 2.2 | 12.1 | 1.4 |
| 90 | 15.2 | 2.8 | 11.0 | 2.0 | 8.8 | 1.6 | 9.8* | 0.6 | 7.4* | 1.2 |
| 120 | 15.5 | 3.6 | 10.8 | 1.7 | 6.5* | 1.2 | 4.8* | 0.3 | 3.0* | 0.1 |
| 180 | 14.7 | 3.1 | 11.0 | 1.6 | 6.5* | 1.2 | 4.0 | 0.6 | 2.6* | 0.2 |

All animals were fed at 0 minutes and dosed at 60 minutes.
*p < 0.05 compared to 60 minute value in the same curve by two-tailed paired two-sample t-test.

Experiments to Measure the Duration of Action of LUM001:

In dogs a single experimental meal produces a postprandial rise in SBA that is elevated to a peak at 1 hour after feeding and constant for an additional 3 hours. Previous experiments (2) indicate that LUM001 remains active for more than 4.5 hours. To measure the duration of action of an ASBT inhibitor using postprandial SBA levels requires that in the control situation the SBA levels remain elevated and constant for the entire period of compound action, or that the compound be administered long before the postprandial rise occurs, and remain active in the empty digestive system for long periods before feeding. Accordingly, two alternative methods were used to provide a window of constant SBA elevation that could be used to measure the duration of action of ASBT inhibitors.

Method 1: Two Meals for Extended SBA Elevation:

LUM001 was administered at 0.05 and 0.2 mg/kg, p.o. to 6 dogs 1 hr after feeding them a meal. At 4 hours after the meal was offered, a second meal of ½ the size of the first meal was offered. It too was consumed as promptly and thoroughly as the first meal, and provided an extended, constant SBA plateau. Blood samples for SBA measurement were taken at 0, 1, 1.5, 2, 4, 4.5, 5, 5.5, 6, 6.5, 7, 7.5 and 8 hours from the time of offering the first meal. Mean SBA levels were compared to the mean SBA level at 1 hour, each dog serving as its own control. The end of activity is considered to occur at time point at which the mean SBA value is not significantly lower than the 1 hr mean value.

TABLE 2
Duration of Action of LUM001 on Dog Serum Bile Acids I
Serum Bile Acid (μg/ml)

| SD-5613 | Water, | | 0.05 mg/kg, | | 0.2 mg/kg, | |
|---|---|---|---|---|---|---|
| Time | n = 6 | | n = 6 | | n = 6 | |
| (hr) | Mean | SEM | Mean | SEM | Mean | SEM |
| 0 | 2.5 | 0.5 | 1.4 | 0.1 | 1.3 | 0.1 |
| 1 | 13.1 | 1.3 | 9.2 | 1.8 | 11.1 | 1.5 |
| 1.5 | | | 9.6 | 2.0 | 9.1 | 0.6 |
| 2 | 14.6 | 1.2 | 6.7 | 0.6 | 3.8* | 0.4 |
| 3 | 14.4 | 1.7 | | | | |
| 4 | 14.8 | 1.2 | 5.1* | 0.7 | 2.5* | 0.4 |
| 4.5 | 16.8 | 1.5 | 6.4 | 0.7 | 2.5* | 0.6 |
| 5 | 15.8 | 2.0 | 7.0 | 0.7 | 3.1* | 0.4 |
| 6 | 15.5 | 2.1 | 7.0 | 0.9 | 3.6* | 0.7 |
| 7 | 14.4 | 2.5 | 7.4 | 0.8 | 3.9* | 0.5 |
| 8 | 13.3 | 1.5 | 6.5 | 1.1 | 5.8* | 0.8 |

All animals were fed a full meal at 0 hour, dosed orally with the compound at 1 hour and then fed an additional one-half meal at 4 hours. *, p<0.05 compared to the mean value in the same curve at 1 hour by two-tailed paired two-sample t-test.

Method 2: One Meal and Extended Interval Between Dosing and Feeding:

Alternatively, 6 dogs were dosed with water or LUM001, at 0.05 mg/kg, p.o. at 1.5 hours prior to being fed, or 0.05, or 0.2 mg/kg, at 2 hours prior to feeding. This moved the elevated SBA plateau out in time from the dose point. Blood samples for SBA measurement were taken immediately before dosing (0 or 0.5 hr), at feeding (2 hr), 2.5, 3, 4 and 5 hours after feeding. This allowed detection of activity out to 5.5 and 6 hours after dosing without feeding the dogs a second time. Mean SBA levels were compared to the corresponding mean SBA levels in water treated controls. The end of activity is considered to occur at the first time point at which the mean SBA value is not significantly lower than the corresponding control mean value.

TABLE 3
Duration of Action of LUM001 on Dog Serum Bile Acids II
Serum Bile Acid (μg/ml)

| | Dosing Time | | | | | | | |
|---|---|---|---|---|---|---|---|---|
| | | | 0.5 hr | | 0 hr | | 0 hr | |
| | Feeding time | | | | | | | |
| | 2 hr | | 2 hr | | 2 hr | | 2 hr | |
| SD-5613 | Water, | | 0.05 mg/kg, | | 0.05 mg/kg, | | 0.2 mg/kg, | |
| Time | n = 6 | | n = 9 | | n = 9 | | n = 6 | |
| (hr) | Mean | SEM | Mean | SEM | Mean | SEM | Mean | SEM |
| 0 | | | | | 1.7 | 0.1 | 1.3 | 0.1 |
| 0.5 | | | 1.8 | 0.3 | | | | |
| 2 | 2.0 | 0.3 | 1.7 | 0.1 | 2.0 | 0.5 | 1.7 | 0.3 |
| 2.5 | 6.9 | 2.1 | 2.5 | 0.6 | | | | |
| 3 | 17.8 | 3.2 | 9.7 | 2.6 | 9.0* | 1.4 | 4.1* | 0.6 |
| 4 | 15.5 | 3.6 | 12.4 | 2.0 | 10.8 | 1.2 | 6.5* | 0.8 |
| 5 | 14.7 | 3.1 | 11.6 | 2.4 | 10.6 | 0.9 | 7.9* | 1.1 |

*p < 0.05 vs water treatment by two-tailed two-sample t-test without assuming equal variances.

Conclusion:

In the dog SBA model, the ED₅₀ dose (0.2 mg/kg) of LUM001 administered orally 1 hour after feeding significantly lowered serum bile acid levels within 30 minutes of dosing and these levels remained significantly lowered for at least 6 hours. By comparison, a threshold dose of 0.05 mg/kg significantly lowered SBA levels within approximately 1 to 2 hours after dosing but the significant decrease was not sustained beyond 3 hours after dosing. Increasing the dose above the ED₅₀ level to 1 mg/kg did not shorten the onset time to significant SBA lowering and still sustained a maximal suppression for 2 hours after dosing. When LUM001 was administered 2 hours prior to feeding, a dose of 0.2 mg/kg was required to produce a significant effect that was sustained for at least 2-3 hours after feeding. The results from these studies indicate that the presence of food in the GI tract has a significant impact on the pharmacodynamic activity of the ASBT inhibitor, most likely by altering the residence time of the drug in the small intestine.

### Example 13

### Animal Efficacy Study on Oral Dose of LUM001 Compared to Cholestyramine on Serum Bile Acids in Dogs

Test Compound:

LUM001

Dosage Preparation and Administration:

LUM001 was dissolved in 0.2% Tween 80 at concentrations that required the administration of 0.2 ml dosing solution/kg of body weight at each dose tested. Cholestyramine was suspended in water at concentrations that required the administration of 2.5 ml/kg (500 mg/kg) and 1 ml/kg (200 mg/kg). The appropriate volume of solution formulation for each animal was placed into a gelatin capsule, Torpac Inc., size 13, Batch 594, East Hanover, N.J. and administered per os.

Experiments to Measure Inhibition of the Postprandial Rise in SBA:

Test compounds were administered as single oral doses to groups of dogs, 3 to 9 dogs per group at varying dosages. LUM001 and cholestyramine were administered in solution 30 minutes prior to feeding and blood samples collected at 30-minute intervals for four hours after feeding. LUM001 was given at 0, 0.02, 0.05, 0.2, 0.6, 2, 5 or 15 mg/kg. Cholestyramine was administered at 200 or 500 mg/kg. The postprandial SBA AUC (0-240 min) was measured: postprandial serum total bile acids were determined from the area under the four-hour curve (AUC). No dog received any one compound dosage more than once.

Serum Total Bile Acid Measurement:

SBA was measured by an enzymatic assay. SBA values are expressed as μg of total bile acids/ml of serum.

Results:

LUM001 significantly decreased serum bile acids. LUM001 showed superior inhibition of postprandial serum bile acids AUC, at a lower dose, as compared to that of cholestyramine (FIG. 3A and FIG. 3B, respectively. Note: data are mean±SEM, n=3-9, *=p<0.05 vs. vehicle group.).

### Example 14

### Animal Efficacy Study on Oral Dose of LUM001 on Fecal Bile Acids in Hamsters

Test Compound:

LUM001

Animal Handling, Dosing and Sample Collection:

Male golden Syrian hamsters (126-147 gm) were obtained from Charles Rivers Laboratories and were single housed in a constant temperature environment with alternating 12-hour light and dark cycles. Hamsters were fed Teklad 7001 rodent meal chow at libitum for two weeks before the experimental studies began and switched to Teklad 7001 rodent meal chow supplemented with 0.24% cholesterol on day one of the 28-day experiment. LUM001 was dissolved in an aqueous solution of 0.2% (w/v) Tween 80 and administered Q.D. by intragastric gavage between 9 a.m. and 10 a.m. each morning using a syringe fitted with a flexible feeding tube. Blood samples were collected after 14- and 28-day treatment periods by orbital sinus and cardiac puncture, respectively. Hamsters were anesthetized but not fasted prior to blood collections. Fecal samples were collected during a 48-hour period at the end of days 14 and 28 (i.e., days 13-14 and 27-28, respectively).

Fecal Bile Acid Measurement:

Fecal samples were collected to determine the fecal bile acid (FBA) concentration for each animal. The separate collections from each hamster were weighed and homogenized with distilled water with a Polytron tissue processor (Brinkman Instruments) to generate a homogeneous slurry. Fecal homogenate (1.4 grams) was extracted with 2.6 mL of a solution containing tertiary butanol:distilled water in the ratio of 2:0.6 [final concentration of 50% (v/v) tertiary butanol] for 45 minutes in a 37° C. water bath and subjected to centrifugation for 13 minutes at 2000×g. The concentration of bile acids (mmolesgram homogenate) was determined using a 96-well enzymatic assay system (6, 7). Aliquots of the fecal extracts (20 μl) were added to two sets of triplicate wells in a 96-well assay plate. A standardized sodium taurocholate solution and a standardized fecal extract solution (previously made from pooled samples and characterized for its bile acid concentration) were also analyzed for assay quality control. Aliquots of 90 mM sodium taurocholate (20 ml), were serially diluted to generate a standard curve containing 30-540 nmoles/well. A 230 ml aliquot of reaction mixture containing 1M hydrazine hydrate, 0.1 M pyrophosphate and 0.46 mg/ml NAD was added to each well. Subsequently, a 50 ml aliquot of either 3a-hydroxysteroid dehydrogenase enzyme (HSD; 0.8 units/ml) or assay buffer (0.1 M sodium pyrophosphate) was then added to one each of the two sets of triplicates. Following 60 minutes of incubation at room temperature, the optical density at 340 nm was measured and the mean of each set of triplicate samples was calculated. The difference in optical density±HSD enzyme was used to determine the bile acid concentration (mM) of each sample based on the sodium taurocholate standard curve. The bile acid concentration of the extract (mmolesgram homogenate), the total weight of the fecal homogenate (grams) and the body weight of the hamsters (g) were used to calculate the corresponding FBA concentration in mmoles/day/kg body weight for each animal. All reagents used for the assay were obtained from Sigma Chemical Co., St. Louis, Mo. (HSD—catalog # H-1506; NAD—catalog # N1636; sodium taurocholate—catalog # T-4009).

TABLE 4
Pharmacological Evaluation of LUM001 Administered to
Hamsters for 28 Days

| | | | LUM001 (mg/kg/day) | | | | | |
|---|---|---|---|---|---|---|---|---|
| Parameter | Vehicle | | 5 | | 15 | | 50 | |
| Body Weight | 131 ± | 3 | 130 ± | 5 | 136 ± | 4 | 130 ± | 4 |
| (BW) (g) | | | | | | | | |
| Weight-Adjusted | 18 ± | 4 | 60 ± | 8* | 66 ± | 6* | 94 ± | 14* |
| Fecal Total | | | (+233) | | (+267) | | (+422) | |
| Bile Acids | | | | | | | | |
| Excretion | | | | | | | | |
| (μmol/day/kg) | | | | | | | | |

All values shown are mean ± SEM, n = 10; (% change from vehicle group); *= p < 0.05 vs. vehicle group, HMG-CoA = 3-hydroxy-3-methylglutaryl coenzyme A.

### Example 15

### Animal Efficacy Study on Oral Dose of LUM001 on Fecal Bile Acids in Rats

Test Compound:

LUM001

Animal Handling, Dosing and Sample Collection:

Male Wistar rats (Charles River Laboratories) weighing 275-300 grams were single-housed in a constant temperature environment with alternating 12 hour light and dark cycles. All animals had continuous access to a commercial rodent diet as well as water. In each study rats were randomly assigned to either vehicle or treatment groups and were administered intragastric doses (Q.D. gavage) of drug dissolved in aqueous 0.2% (v/v) Tween 80 (2 ml kg body weight). The animals were dosed in the morning between 9:00 and 10:00 a.m. for four consecutive days. Fecal samples were collected on papers underneath each cage during the final 48 hour period of the study and analyzed for bile acid content.

Fecal Bile Acid Measurement:

Cage papers containing the 48-hour fecal samples were collected at approximately 9:00 a.m. on the final day and used to determine the individual fecal bile acid (FBA) concentration for each animal. Fecal samples from each rat were weighed and a weight of distilled water (1 gram/mL) equal to 2 times the total weight of feces was added to each sample container (e.g., 20 mL water to 10 grams feces). The containers were stored overnight at 4° C. Each sample was homogenized for approximately 45 seconds using a small food processor to yield a homogeneous slurry. 1.4 grams of the homogenate was weighed into 16×100 polypropylene tubes and 2.6 mL of tertiary butanol/distilled water (2:0.6) added to yield a final concentration of 50% (v/v) tertiary butanol in water. The sample was extracted by incubation for 45 minutes in a 37° C. water bath, and subjected to centrifugation at 3000×g for 13 minutes and the supernatant extract collected.

The concentration of bile acids (mmoles/day) in the extract was determined using a 96-well enzymatic assay system (4,5). 20 μl aliquots of each butanol extract was added to two sets of triplicate wells in a 96-well assay plate (one set on each half of the plate). Standardized sodium taurocholate solutions (0.2 and 0.9 mM) and standardized fecal extract solutions (previously made from pooled fecal samples collected from control and drug-treated rats) were analyzed in parallel to provide a bile acid standard curve and internal quality control samples, respectively. 20 μl aliquots of the sodium taurocholate standard were serially diluted to generate a standard curve and were added to two separate sets of triplicate wells.

To each well, 230 μl of reaction mixture containing 1M hydrazine hydrate, 0.1 M pyrophosphate and 0.46 mg/ml NAD was added. To start the reaction, a 50 μl aliquot of either 3a-hydroxysteroid dehydrogenase enzyme (HSD; 0.8 units/ml) or assay buffer (0.1 M sodium pyrophosphate) was added to one set of triplicate wells for each sample, respectively, with the set containing the assay buffer serving as the reaction blank. All reagents were obtained from Sigma Chemical Co., St. Louis, Mo. Following a 60 minute incubation at room temperature, the optical density at 340 nm was measured and the mean of each set of triplicate wells was calculated. The difference in optical density between the corresponding wells containing the HSD enzyme and the wells containing the assay buffer was used to determine the bile acid concentration (mM) of each sample by comparison to the sodium taurocholate standard curve. The bile acid concentration of the extract and the weight of the fecal homogenate (grams) were used to calculate FBA concentration in mmoles/day for each animal. The mean FBA concentration (mmoles/day) of the vehicle group was subtracted from the FBA concentration of individual rats in a treatment group to yield the increase in FBA concentration due to drug treatment for that animal. A mean value for the increase in FBA for each group was determined and compared to the vehicle group used, to determine compound dosing efficacy. Data was the average from the two days of fecal collection (Mean±SEM, n=4-20).

Statistical Analysis:

Due to the increase in variance in proportion to the FBA level, a log transformation was used before fitting a dose-response model. A four parameter logistic curve was fit using non-linear least squares and the EC₅₀ and its approximate standard error reported are those from the least squares fit.

Results:

LUM001 significantly increased fecal bile acids for all doses (FIG. 4).

TABLE 5
Effect of LUM001 on Fecal Bile Acids in Rats (Mean ± SEM)
Q.D. Dose of LUM001 (mg/kg/day)

| | 0 | 0.0006 | 0.002 | 0.003 | 0.008 | 0.016 | 0.04 | 0.08 | 0.2 | 0.4 | 2 |
|---|---|---|---|---|---|---|---|---|---|---|---|
| Fecal | 15.3 ± | 17.1 ± | 16.3 ± | 16.2 ± | 17.3 ± | 17.7 ± | 14.9 ± | 16.8 ± | 24.4 ± | 17.9 ± | 18.0 ± |
| Weight | 0.4 | 1.8 | 1.9 | 1.1 | 0.4 | 0.9 | 0.8 | 0.6 | 3.7 | 1.3 | 0.9 |
| (gm/48 hr) | | | | | | | | | | | |
| Fecal Bile | 19.5 ± | 34.0 ± | 26.6 ± | 31.3 ± | 25.8 ± | 41.6 ± | 44.0 ± | 55.6 ± | 62.7 ± | 62.3 ± | 71.6 ± |
| Acids | 0.6 | 1.3* | 0.7* | .13* | .27 | .10^{†} | .07^{†} | .07^{†} | .09^{†} | .08^{†} | .07^{†} |
| (mmole/24 | | | | | | | | | | | |
| hr) | | | | | | | | | | | |
| Increase | X | 12.1 ± | 4.1 ± | 10.7 ± | 5.9 ± | 24.7 ± | 21.7 ± | 35.2 ± | 40.8 ± | 47.0 ± | 54.6 ± |
| over | | 5.3 | 2.0 | 3.9 | 7.4 | 3.6 | 3.3 | 3.8 | 6.2 | 6.3 | 4.8 |
| Vehicle | | | | | | | | | | | |
| (Delta) | | | | | | | | | | | |
| N | 46 | 4 | 4 | 8 | 4 | 20 | 4 | 20 | 4 | 20 | 16 |

*p < 0.05 vs. vehicle group, Student's 2-tailed T-test.
^{†}p < 0.01 vs. vehicle group, Student's 2-tailed T-test.

### Example 16

### Animal Efficacy Study on Oral Dose of LUM001 on Fecal Bile Acids in Dogs

Test Compound:

LUM001

Animal Handling, Dosing and Sample Collection:

Healthy female and male beagle dogs in the age range of 8 to 10 months were used. The animals were acclimated for at least four weeks before dose administration. Four female and four male dogs per dose group (groups 2-5) were given single daily doses of LUM001 at 1, 4, 12 and 30 mg free form/kg for 13 days. Animals in the control group (group 1) were given the same number of empty capsules as the animals of group 5 to treat all dose groups the same.

Fecal Bile Acid Measurement:

Fecal samples were collected to determine the fecal total bile acid (FBA) concentration for each animal. Fecal collections were made during the final 72 hours of the study, for three consecutive 24-hour periods between 9:00 am and 10:00 am each day, prior to dosing and feeding. The separate daily collections from each dog were weighed, combined and homogenized with distilled water in a food processor to generate a homogeneous slurry. Homogenate (1.4 g) was extracted in a final concentration ratio of 2:0.6 of 50% (v/v) tertiary butanol/distilled water for 45 minutes in a 37° C. water bath and subjected to centrifugation for 13 minutes at 2000×g. The concentration of bile acids (mmolesgram homogenate) was determined using a 96-well enzymatic assay system. Aliquots of the fecal extracts (20 μl) were added to two sets of triplicate wells in a 96 well assay plate. A standardized sodium taurocholate solution and a standardized fecal extract solution (previously made from pooled samples and characterized for its bile acid concentration) were also analyzed for assay quality control. Aliquots of sodium taurocholate (20 μl), were serially diluted to generate a standard curve containing 30-540 nmoles/well. A 230 μl aliquot of reaction mixture containing 1M hydrazine hydrate, 0.1 M pyrophosphate and 0.46 mg/ml NAD was added to each well. Subsequently, a 50 μl aliquot of either 3a-hydroxysteroid dehydrogenase enzyme (HSD; 0.8 units/ml) or assay buffer (0.1 M sodium pyrophosphate) was added to one each of the two sets of triplicates. Following 60 minutes of incubation at room temperature, the optical density at 340 nm was measured and the mean of each set of triplicate samples was calculated. The difference in optical density±HSD enzyme was used to determine the bile acid concentration (mM) of each sample based on the sodium taurocholate standard curve. The bile acid concentration of the extract (mmolesgram homogenate), the total weight of the fecal homogenate (grams) and the body weight of the dogs (kg) were used to calculate the corresponding FBA concentration in mmoles/kg/day for each animal. All reagents used for the assay were obtained from Sigma Chemical Co., St. Louis, Mo. (HSD—catalog # H-1506; NAD—catalog # N1636; sodium taurocholate—catalog # T-4009). A one-tailed, paired Student's t-test was used to determine the statistical significance of changes in FBA concentration in treated animals compared to pretreatment values and between treatment groups.

Preparation of Liver Microsomes:

At the end of the study, animals were anesthetized, their livers removed and flash frozen and stored at −80 C. Homogenates of three gram liver tissue samples were prepared in 25 ml homogenization buffer [0.1 M potassium phosphate buffer, pH 7.2, containing 0.1 M sucrose, 50 mM KCl, 50 mM NaF, 5 mM ethylene glycol-bis((3-aminoethyl ether)-N,N,N′,N′-tetraacetic acid (EGTA), 1 mM EDTA, 3 mM dithiothreitol (DTT), and 1 mM phenylmethylsulfonyl fluoride (PMSF)]. A microsomal fraction was prepared by centrifugation at 10,000×g for ten minutes. The supernatant was subjected to centrifugation at 105,000×g for two hours. The microsomal fraction was resuspended in a 0.1 M Na tetrapyrophosphate buffer (pH 10), with 50 mM NaF, and 1 mM EDTA, and subjected to centrifugation for one hour at 105,000×g. The microsomal fraction was resuspended in the homogenization buffer, and assayed for protein content by Coomassie Protein Plus Assay reagent.

3-Hydroxy-3-Methylglutaryl Coenzyme a (HMG-CoA) Reductase Activity Assay:

Microsomes (200 μg) were preincubated for 15 minutes at 37° C. in a total volume of 225 ml buffer containing 0.1 M potassium phosphate (pH 7.4), 10 mM imidazole, 5 mM DTT, 10 mM EDTA, 3 mM NADP, 12 mM glucose-6-phosphate and 1 unit glucose-6-phosphate dehydrogenase (catalog number G4134, Sigma, St. Louis). The HMG-CoA reductase assay was initiated by the addition of 9 nmoles (0.5 μCi) DL-[3¹⁴C] HMG-CoA (final assay volume was DL-3-[¹⁴C]-250 up. The mixture was incubated for 60 minutes at 37° C. after which the assay was stopped with 25 μl of 6 N HCl. [³H]Mevalonic acid (0.1 μCi), used as an internal standard to correct for incomplete recovery, was added to the reaction mixture along with 3 mg of unlabeled mevalonic acid lactone. The mixture was then incubated for an additional 30 minutes at 37° C. The [¹⁴C]mevalonate that was formed and converted into mevalonic acid lactone during both incubations was isolated by thin layer chromatography. Each lane was scraped into 3 ml of InstaGel XF (Packard, Meriden, Conn.) and read on a Beckman Scintillator Counter. Reagents unless noted otherwise were obtained from Sigma, St. Louis, Mo. Isotopes were obtained from NEN Life Science Products, Boston, Mass.

Cholesterol 7-α Hydroxylase Activity Assay:

Liver microsomes (1 mg) or 7-α-hydroxycholesterol standard solutions were preincubated for five minutes at 37° C. in a rocking water bath with 500 μl 5× buffer, 1.7 ml water, and 50 μl 0.1% (w/v) cholesterol as substrate in excess. The 5× buffer consists of 0.42 M Na₂HPO₄, 0.25 mM NaF, 0.08 M KH₂PO₄, 5 mM EDTA and 10 mM DTT. The reaction tubes were incubated at 37° C. in a rocking water bath throughout the remainder of the assay protocol, with cessation of rocking only for the period of time needed to add solutions. After the preincubation period, 250 μl of 10 mM NADPH was added and incubated for 5 minutes to allow enzymatic conversion of the cholesterol substrate to 7-α-hydroxycholesterol by endogenous 7-α-hydroxylase. The reaction was stopped by addition of 75 μl of 20% sodium cholate.

After four minutes, 25 μl of 100 μM 20-α-hydroxycholesterol was added to each tube as an internal recovery standard. The tubes were allowed to rock briefly in the water bath to mix, then 40 μl of 25 U/ml cholesterol oxidase was added and the tubes were incubated for ten minutes to convert 7-α-hydroxycholesterol and 20-α-hydroxycholesterol to their ketone forms, 7-α-hydroxycholesten-3-one and 20-α-hydroxycholesten-3-one. After the enzymatic reactions were completed, the ketone products were isolated by four sequential extractions in 20, 10, 10 and 10 ml petroleum ether, respectively. The collected volumes were evaporated to dryness under a flow of nitrogen gas between each extraction step using a heat block set at 50° C. The final dried samples were resuspended in 125 μl mobile phase solution consisting of 70:30 acetonitrile/methanol for analysis by reversed phase HPLC. Chromatography was performed on a 4.6×250 mm Beckman Ultrosphere ODS reverse phase column in mobile phase solution. The analyte, 7-α-hydroxy-4-cholesten-3-one, is quantified by absorption at 254 nm using the internal standard 20-α-hydroxy-4-cholesten-3-one to control for extraction efficiency.

TABLE 6
Pharmacological Evaluation of LUM001 Administered to
Dogs for 14 Days

| | Vehicle | LUM001 (mg/kg/day) | | | |
|---|---|---|---|---|---|
| Parameter | Control | 1 | 4 | 12 | 30 |
| Weight- | 59 ± 8 | 96 ± 9 | 140 ± 6 | 152 ± 13 | 167 ± 15 |
| Adjusted Fecal | | (+63) | (+137) | (+158) | (+183) |
| Total Bile | | | | | |
| Acids Excretion | | | | | |
| (μmol/day/kg) | | | | | |
| Cholesterol | 89 ± 8 | 114 ± 18 | 179 ± 22 | 284 ± 41 | 425 ± 58 |
| 7a-hydroxylase | | (+28) | (+101) | (+219) | (+378) |
| (pmol/mg/min) | | | | | |
| HMG-CoA | 72 ± 14 | 164 ± 20 | 246 ± 30 | 395 ± 46 | 544 ± 33 |
| Reductase | | (+128) | (+242) | (+449) | (+656) |
| (pmol/mg/min) | | | | | |

All values shown are mean ± SEM, except cholesterol which are mean ± SD; n = 8; (% change from vehicle group).

### Example 17

### Animal Efficacy Study on Oral Dose of LUM001 on Fecal Bile Acids in Monkeys

Test Compound:

LUM001

Animal Handling, Dosing and Sample Collection:

Fifteen experimentally naive male rhesus monkeys were utilized in this study. These animals were 2.1 to 4.2 years of age and weighed between 2.8 to 4.3 kg on the day prior to treatment initiation. HARLAN TEKLAD PRIMATE DIET® (Certified) was provided daily in amounts appropriate for the size and age of the animals. The diet was supplemented with fruit or vegetables 2-3 times weekly. Small bits of fruit, cereal or other treats were given to the animals following dose administration and periodically as part of facility's environmental enrichment program. Beginning prior to treatment initiation, only non-fat treats were provided to the animals (e.g., standard primate treats including peanut butter, sunflower seeds, baby food, nuts, pudding, and worms were specifically not permitted). Tap water was available ad libitum via an automatic watering device. Each animal was administered a dose of the LUM001 contained within gelatin capsules or the control article (empty gelatin capsules) once daily for 14 consecutive days. Fecal samples for determination of bile acid content were collected over approximately 24-hour periods beginning three days prior to treatment initiation and continuing until scheduled termination of dosing on Day 15. Samples were stored at −70 C until analysis.

Fecal Bile Acid Measurement:

Fecal samples were collected to determine the fecal total bile acid (FBA) concentration for each animal. Fecal collections were made during the final 72 hours of the study, for three consecutive 24-hour periods between 9:00 am and 10:00 am each day, prior to dosing and feeding. The separate daily collections from each dog were weighed, combined and homogenized with distilled water in a food processor to generate a homogeneous slurry. Homogenate (1.4 g) was extracted in a final concentration ratio of 2:0.6 of 50% (v/v) tertiary butanol/distilled water for 45 minutes in a 37° C. water bath and subjected to centrifugation for 13 minutes at 2000×g. The concentration of bile acids (mmolesgram homogenate) was determined using a 96-well enzymatic assay system. Aliquots of the fecal extracts (20 μl) were added to two sets of triplicate wells in a 96 well assay plate. A standardized sodium taurocholate solution and a standardized fecal extract solution (previously made from pooled samples and characterized for its bile acid concentration) were also analyzed for assay quality control. Aliquots of sodium taurocholate (20 μl), were serially diluted to generate a standard curve containing 30-540 nmoles/well. A 230 μl aliquot of reaction mixture containing 1M hydrazine hydrate, 0.1 M pyrophosphate and 0.46 mg/ml NAD was added to each well. Subsequently, a 50 μl aliquot of either 3α-hydroxysteroid dehydrogenase enzyme (HSD; 0.8 units/ml) or assay buffer (0.1 M sodium pyrophosphate) was added to one each of the two sets of triplicates. Following 60 minutes of incubation at room temperature, the optical density at 340 nm was measured and the mean of each set of triplicate samples was calculated. The difference in optical density±HSD enzyme was used to determine the bile acid concentration (mM) of each sample based on the sodium taurocholate standard curve. The bile acid concentration of the extract (mmolesgram homogenate), the total weight of the fecal homogenate (grams) and the body weight of the dogs (kg) were used to calculate the corresponding FBA concentration in mmoles/kg/day for each animal. All reagents used for the assay were obtained from Sigma Chemical Co., St. Louis, Mo. (HSD—catalog # H-1506; NAD—catalog # N1636; sodium taurocholate—catalog # T-4009). A one-tailed, paired Student's t-test was used to determine the statistical significance of changes in FBA concentration in treated animals compared to pretreatment values and between treatment groups.

TABLE 7
Pharmacological Evaluation of LUM001 Administered to
Rhesus Monkeys for 14 Days.

| Parameter | Group | Prestudy | | Day 8 | Day 15 | |
|---|---|---|---|---|---|---|
| Fecal Total | Control | 5 ± | 0.7 | N.D. | 6 ± | 0.6 |
| Bile Acids | | | | | | |
| (μmol/day/kg) | 5 mg/kg | 6 ± | 1 | N.D. | 23 ± | 7 |
| | 50 mg/kg | 6 ± | 2 | N.D. | 63 ± | 14 |

All values shown are mean ± SD, except fecal bile acids, which are mean ± SE. n = 5. N.D. = sample not analyzed.

### Example 18

### Pharmacokinetics Demonstrating Nonsystemic Absorption of LUM001 in Rats and Dogs

Test Compound:

LUM001

Animal Handling, Dosing and Sample Collection:

Groups of fasted Sprague Dawley rats (n=6/sex/group) were administered LUM001 as a single oral solution dose of either 1, 5 or 30 mg LUM001 free form/kg. The dose of 5 mg LUM001 free form/kg was also administered to rats in a fed state to evaluate the effect of food on plasma concentrations of LUM001 free form. Since LUM001 is a chloride salt, 105% of the amount of LUM001 is required to deliver the stated dose of the free form. The oral dose was delivered to the rat in Tween 80Milli-Q water (0.2% Tween, v/v) and was prepared on the day of dosing.

Pharmacokinetics Measurement:

Blood samples of approximately 1 mL (n=2/sex/timepoint/treatment group) were collected by retro-orbital bleed into chilled tubes containing heparin at timepoints 0 (predose), 0.25, 0.5, 1, 1.5, 2, 3, 5 and 8 hours. The plasma samples were prepared by centrifugation of blood within 30 minutes after sample collection. All samples were stored at −20° C.±5° C. until analysis to study the relative exposures of LUM001 free form after oral solution dosing. The concentration of LUM001 free form in plasma were determined using an LCMSMS method. The assay sensitivity was 2.38 ng/mL.

Means and standard errors of the means (SEM) were calculated for plasma concentrations at each time point. Mean values are significant to three figures and SEM values are significant to the same decimal place as the corresponding mean. Concentration values less than the assay sensitivity (2.38 ng LUM001 free form/mL) were reported as zero. The area under the plasma concentration-time curve (AUC) was calculated from time 0 to 8 hours using a non-compartmental model in WinNonlin (1). The bioavailability (% BA) of LUM001 after oral administration of LUM001 was calculated according to the equation below:

% BA=[AUC oral (0-8)/AUC IV(0-8)/Dose oral/Dose IV]×100.

TABLE 8
Pharmacokinetic Parameters after Oral Gavage Administration of
LUM001 to Rats.

| Dose | Tₘₐₓ | Cₘₐₓ | AUC_{(0-8 h)} | BA^{b} |
|---|---|---|---|---|
| (mg/kg)^{a} | (h) | (ng/mL) | (ng · h/mL) | (%) |
| 1 | NC | NC | NC | <0.1 |
| 5 | 1.00 | 2.45^{c} | 6.58 | 0.1 |
| 5 (fed) | 0.250 | 3.58 | 12.6 | 0.2 |
| 30 | 0.500 | 5.43 | 4.16 | <0.1 |

Data are derived from mean plasma concentrations from 6 male and 6 female Sprague Dawley rats at each time-point. Tₘₐₓ = time to maximum plasma concentration; Cₘₐₓ = maximum plasma concentration; AUC_{(0-8 h)} = area under the plasma concentration-time curve from time 0 to 24 hours post-dose; BA = bioavailability; NC = not calculable, plasma concentrations below the assay sensitivity limit (2.5 ng/mL); ^{a}Expressed as mg LUM001 free form/kg; ^{b}Bioavailability calculated using the AUC_{(0-8 h)} after IV administration of 5 mg/kg LUM001; ^{c}Mean concentration is less than the assay sensitivity limit (2.5 ng/mL)

Three fasted female Beagle dogs were administered LUM001 either as an oral solution at doses of 1 or 7.5 mg LUM001 free form/kg or in an oral capsule at a dose of 7.5 mg LUM001 free form/kg in a cross-over design. A dose of 7.5 mg LUM001 free form/kg was also administered as an oral solution to fed beagle dogs to evaluate the effect of food on plasma concentrations of LUM001.

Blood samples of approximately 3 mL was collected by venipuncture or indwelling catheter into chilled tubes containing heparin at the following timepoints: 0 (predose), 0.5, 1, 1.5, 2, 3, 5, 6, 8, 10, and 24 hours. The plasma samples were prepared by centrifugation of blood within 30 minutes after sample collection. All samples were stored at −20° C.±5° C. until analysis to study the relative exposures of LUM001 free form after capsule or oral solution dosing.

TABLE 9
Pharmacokinetic Parameters after Oral Solution and Capsule
Administration of LUM001 to Female Beagle Dogs

| Dose | Tₘₐₓ | Cₘₐₓ | AUC_{(0-24 h)} | BA^{b} |
|---|---|---|---|---|
| (mg/kg)^{a} | (h) | (ng/mL) | (ng · h/mL) | (%) |
| 1 | NC | NC | NC | <0.1 |
| 7.5 | NC | NC | NC | <0.1 |
| 7.5 (fed) | 0.500 | 1.28^{c} | 3.90 | <0.1 |
| 7.5 | 5.00 | 4.24 | 33.7 | <0.1 |
| (capsule) | | | | |

Data are derived from mean plasma concentrations from 3 female Beagle dogs. Tₘₐₓ = time to maximum plasma concentration; Cₘₐₓ = maximum plasma concentration; AUC_{(0-24 h)} = area under the plasma concentration-time curve from time 0 to 24 hours post-dose; BA = bioavailability; NC = not calculable, plasma concentrations below the assay sensitivity limit (2.5 ng/mL); ^{a}Expressed as mg LUM001 free form/kg
^{b}Bioavailability calculated using the oral AUC_{(0-8 h)} and the AUC_{(0-8 h)} after IV administration of 7.5 mg/kg LUM001; ^{c}Mean concentration is less than the assay sensitivity limit (2.5 ng/mL).

### Example 19

### Toxicokinetics Demonstrating Nonsystemic Absorption of LUM001 in Rats and Dogs

Test Compound:

LUM001

Animal Handling, Dosing and Sample Collection:

Charles River CD IGS rats were assigned to treatment groups (9sex/toxicokinetic group) and were administered daily doses of 0, 5, 30, 75, and 150 mg/kg (males) or 0, 5, 30, 150, and 500 mg/kg (females). LUM001 was administered by once daily oral gavage in distilled water and fresh dosing solutions were prepared weekly. All animals were dosed with 10 mL/kg/day based on the most recently determined body weights. On Day 1 and during Week 12, approximately 1 mL of venous blood was collected in heparinized tubes from the retro-orbital venous plexus. Animals were anesthetized with CO2-O2 and blood samples were collected at 1, 2, 3, 5, 8, and 24 hours post-dose. Survival permitting, the first 3 animals/sex dose group were bled at 1 and 5 hours post-dose, the second 3 animals/sex/dose group were bled at 2 and 8 hours post-dose, and the last 3 animals/sex/dose group were bled at 3 and 24 hours post-dose. Blood collection tubes were kept on ice during the collection period after which they were centrifuged and the plasma was harvested within approximately 60 minutes of blood collection. Plasma samples were stored at approximately −20° C. or lower until analyzed.

Pharmacokinetics Measurement:

The concentration of LUM001 free form in plasma were determined using an LCMSMS method. The assay sensitivity was 4.75 ng/mL. Means and standard errors of the means (SEM) were calculated for plasma concentrations at each time point. Mean values are significant to three figures and SEM values are significant to the same decimal place as the corresponding mean. Concentration values less than the assay sensitivity (4.75 ng LUM001 free form/mL) were reported as zero. The observed peak plasma concentrations (Cmax) of LUM001 free form, time to reach peak plasma concentration (Tmax) and areas under the plasma concentration-time curve (AUC₀₋₂₄) were calculated by the TOXAUC computer program (1). For calculation purposes the 0 hr plasma concentrations were set to zero on Day 1 and to the concentrations at 24 hr post dose on Day 78. The TK parameters were calculated from mean data for each group of rats. The bioavailability (% BA) of LUM001 after oral administration of LUM001 was calculated according to the equation below:

% BA=[AUC oral (0-8)/AUC IV(0-8)/Dose oral/Dose IV]×100

TABLE 10
Toxicokinetic Parameters of LUM001 Free Form in the 13-Week
Oral Gavage Toxicity Study in Rats.

| | Dose | | Tₘₐₓ | Cₘₐₓ (ng/mL) | | AUC_{(0-24 h)} | BA |
|---|---|---|---|---|---|---|---|
| Day | (mg/kg)^{a} | Sex | (h) | Mean | SEM | (ng · h/mL) | (%)^{b} |
| 1 | 5.0 | F | NC | NC | NA | NC | <0.1 |
| | | M | NC | NC | NA | NC | <0.1 |
| | 30.0 | F | NC | NC | NA | NC | <0.1 |
| | | M | 2.00 | 8.59 | 8.59 | 15.1 | — |
| | 75.0^{c} | M | 5.00 | 2.55^{d} | 2.55 | 6.37 | <0.1 |
| | 150 | F | 2.00 | 42.4 | 38.5 | 106 | — |
| | | M | 3.00 | 32.1 | 32.1 | 96.3 | — |
| | | Comb | 2.00 | 37.3 | 19.6 | 101 | <0.1 |
| | 500^{c} | F | 3.00 | 9.99 | 7.61 | 90.8 | <0.1 |
| 78 | 5.0 | F | NC | NC | NA | NC | <0.1 |
| | | M | 2.00 | 5.80 | NA | 11.1 | — |
| | 30.0 | F | 3.00 | 3.95^{d} | 2.08 | 12.4 | — |
| | | M | 3.00 | 4.75^{d} | 4.75 | 11.4 | — |
| | | Comb | 3.00 | 4.35^{d} | 2.32 | 11.7 | <0.1 |
| | 75.0^{c} | M | 8.00 | 5.71 | 3.47 | 61.4 | <0.1 |
| | 150 | F | 2.00 | 22.2 | 3.97 | 124 | — |
| | | M | 1.00 | 28.3 | NA | 141 | — |
| | | Comb | 1.00 | 24.1 | 9.27 | 132 | <0.1 |
| | 500^{c} | F | 3.00 | 62.9 | NA | 470 | <0.1 |

Parameters are derived from mean plasma concentrations of 3 male and 3 female Sprague Dawley rats at each time-point. Tₘₐₓ = time to maximum plasma concentration; Cₘₐₓ = maximum plasma concentration; SEM = standard error of the mean; AUC_{(0-24 h)} = area under the plasma concentration-time curve from time 0 to 24 hours post-dose; BA = bioavailability; F = female; M = male; Comb = combined sexes; NC = not calculable, plasma concentrations below the lower limit of quantitation; NA = not applicable; — = not calculated; ^{a}Expressed as mg LUM001 free form/kg; ^{b}Bioavailability calculated using an AUC_{(0-24 h)} of 4320 ng · h/mL after IV administration of 3 mg/kg LUM001; ^{c}Males only; ^{d}Mean concentration is less than the lower limit of quantitation (4.75 ng/mL); ^{e}Females only.

Six- to seven-month old Beagle dogs of body weights of 4.9-9.5 kg (4/sex/group) orally via capsules at 0, 5, 20 or 100 mg/kg for 13 consecutive weeks. Plasma levels of LUM001 (free form) were evaluated on Days 1 and 91. LUM001 was administered by once daily oral gelatin capsules. is a chloride salt therefore, 105% of a stated dose is given to provide the appropriate amount of compound for dosing. On study Day 1 and Day 91, approximately 2 mL of venous blood was collected in heparinized tubes from the cephalic vein of each animal at 1, 2, 3, 5, 8, and 24 hours post-dose. Blood collection tubes were mixed and placed on ice during the collection period after which they were centrifuged and the plasma was harvested into cryotubes within approximately 60 minutes of blood collection. Plasma samples were stored at approximately −70° C. or lower until analyzed.

The concentration of LUM001 free form in plasma were determined using an LCMSMS method. The assay sensitivity was 5.00 ng/mL for a 100 mL sample.

TABLE 11
Toxicokinetic Parameters of LUM001 Free Form in the 13-Week Oral
Capsule Toxicity Study in Dogs.

| | | | | | Cₘₐₓ | | | AUC_{(0-24 h)} | | | |
|---|---|---|---|---|---|---|---|---|---|---|---|
| | Dose | | Tₘₐₓ (h) | | (ng/mL) | | | (ng · h/mL) | | AUC/ | BA |
| Day | (mg/kg)^{a} | Sex | Mean | SEM | Mean | SEM | Cₘₐₓ/Dose | Mean | SEM | Dose | (%)^{b} |
| 1 | 5 | F | 1.25 | 0.479 | 9.34 | 3.63 | 1.87 | 20.9 | 8.98 | 4.17 | <0.1 |
| | | M | 1.75 | 0.250 | 8.79 | 1.45 | 1.76 | 14.6 | 5.29 | 2.92 | <0.1 |
| | | Comb | 1.50 | 0.267 | 9.07 | 1.81 | 1.81 | 17.7 | 4.97 | 3.55 | 0.1 |
| | 20 | F | 2.00 | 0.00 | 25.3 | 5.69 | 1.27 | 48.5 | 10.1 | 2.43 | <0.1 |
| | | M | 2.25 | 0.250 | 24.8 | 8.12 | 1.24 | 96.7 | 52.9 | 4.84 | <0.1 |
| | | Comb | 2.13 | 0.125 | 25.1 | 4.59 | 1.25 | 72.6 | 26.5 | 3.63 | 0.1 |
| | 100 | F | 1.50 | 0.289 | 117 | 29.7 | 1.17 | 344 | 116 | 3.44 | <0.1 |
| | | M | 1.25 | 0.250 | 64.8 | 28.7 | 0.648 | 134 | 56.2 | 1.34 | <0.1 |
| | | Comb | 1.38 | 0.183 | 90.7 | 21.5 | 0.907 | 239 | 71.5 | 2.39 | <0.1 |
| 91 | 5 | F | 0.00 | 0.00 | 0.00 | 0.00 | 0.00 | 0.00 | 0.00 | 0.00 | <0.1 |
| | | M | 0.75 | 0.479 | 3.50^{c} | 2.09 | 0.700 | 3.50 | 2.09 | 0.700 | <0.1 |
| | | Comb | 0.375 | 0.263 | 1.75^{c} | 1.17 | 0.350 | 1.75 | 1.17 | 0.350 | <0.1 |
| | 20 | F | 2.00 | 0.00 | 25.0 | 7.45 | 1.25 | 53.9 | 16.1 | 2.70 | <0.1 |
| | | M | 1.75 | 0.250 | 22.6 | 6.61 | 1.13 | 47.6 | 13.7 | 2.38 | <0.1 |
| | | Comb | 1.88 | 0.125 | 23.8 | 4.63 | 1.19 | 50.8 | 9.83 | 2.54 | 0.1 |
| | 100 | F | 1.75 | 0.250 | 182 | 18.1 | 1.82 | 601 | 75.7 | 6.01 | <0.1 |
| | | M | 2.25 | 0.250 | 97.4 | 10.6 | 0.974 | 405 | 89.6 | 4.05 | <0.1 |
| | | Comb | 2.00 | 0.189 | 140 | 18.7 | 1.40 | 503 | 65.7 | 5.03 | 0.2 |

### Example 20

### A Randomized, Double-Blind, Placebo Controlled, Safety, Tolerability, Pharmacokinetic, and Pharmacodynamic Study of Ascending Multiple Oral Doses of LUM001 in Healthy Adult Subjects

This Phase 1 study was a randomized, double-blind, placebo-controlled study of ascending multiple oral doses of LUM001 in healthy, adult subjects. This study was conducted at a single center. There were 13 LUM001 dosing panels: 10, 20, 60, 100, and 20 mg every morning (qAM) (2) (i.e., the regimen was tested a second time in the study), 5 mg every evening (qPM), 0.5, 1, 2.5, 5, 2.5 (2), 5 (2), and 0.5 to 5 mg qAM dose titration. Most of the dosing panels included subjects treated with matching placebo. No subject participated in more than 1 dosing panel. Subjects were randomized into the study by dosing panel, and all subjects within a dosing panel received study medication at approximately the same time each day. Safety was reviewed for each panel before subsequent panels were initiated. A total of 167 subjects were treated for 28 days, 147 with LUM001 and 20 with placebo.

During the Pretreatment Screening Period (Days −31 through −4), subjects were seen on an outpatient basis to determine study eligibility based on enrollment criteria. On Day −3, eligible subjects were admitted to the research facility during the morning. Subjects were confined to the research facility from Days −3 to 30. During the entire period of confinement to the research facility, subjects received a study diet composed of 35% of caloric intake from fat, 15% from protein, and 50% from carbohydrates. The total daily caloric intake (based on the subject's weight) was fixed and divided into 3 equicaloric equal-fat-containing meals. The fixed fat composition and caloric content of the diet was designed to reduce inter- and intra-subject variability of serum total bile acids (SBA) and FBA, lipid parameters, and fat absorption parameters.

On Day 1 of the Treatment Period, subjects who qualified for enrollment were randomized in the order in which they were admitted to the research facility. On the same day, subjects began receiving study medication. Subsequent analyses included clinical and laboratory safety assessments and profiles of PK, PD, and lipid parameters. The safety and tolerability of LUM001 were evaluated by physical examination, ascertainment of adverse signs and symptoms, and clinical laboratory studies. Blood and urine samples were obtained for measurement of LUM001 plasma, whole blood, and urine concentrations for subsequent PK analyses. Efficacy responses included measuring FBA as a surrogate marker for the inhibition of intestinal bile acid transport. For the qAM dosing panels, LUM001 or placebo was administered each day of the treatment period (28 days) immediately prior to the morning meal at approximately 08:00 and after any necessary blood work was drawn.

Serum Bile Acid (SBA) Analysis:

On Day −1, blood was drawn for baseline SBA at approximately 30 minutes before and after breakfast and 30 minutes after lunch and dinner. During the treatment period, samples were obtained on day 14 (FIG. 5; *p<0.05; **p<0.01 compared to placebo) at −30, 30, 60 120, and 240 minutes after each of the 3 daily meals for analysis. For each sample, approximately 3 mL of venous blood were collected by venipuncture or saline lock.

SBA were analyzed as part of the routine clinical analysis of the serum samples collected at each time point.

Fecal Bile Acid Analysis:

Fecal samples were collected for all panels except the dose-titration panel, 2.5 (2) and 5 mg (2), on Days 9 through 14 and 23 through 28 (data shown in FIG. 6; *p<0.05; **p<0.01 compared to placebo). Twenty-four hour FBA excretions were quantified by Pharmacia for Days 9 through 14 and 23 through 28. Feces were collected in a 24-hour collection container beginning at 08:00 and ending 24 hours later. This procedure was followed on Days 9 through 14 and 23 through 28, with new collection containers issued for each 24-hour period. The weight of each 24-hour fecal collection was recorded on the CRFs. Specimens were stored in 24-hour containers, frozen at approximately −80° C. prior to analysis.

An aliquot for each 24-hour fecal sample collected on Days 23 through 28 was combined, homogenized, and analyzed for bile acid species concentrations by ANAPHARM. The fecal bile acid species evaluated include chenodeoxycholic acid, cholic acid, deoxycholic acid, and lithocholic acid.

TABLE 12
Mean (μmole/24 hr) Daily Total Fecal Bile Acids Excretion

| | | LUM001 | | | | | | | | |
|---|---|---|---|---|---|---|---|---|---|---|
| | | | | | | 5 mg | | | | |
| Time | Placebo | 0.5 mg | 1.0 mg | 2.5 mg | 5 mg | qPM | 10 mg | 20 mg | 60 mg | 100 mg |
| Period | (n = 16) | (n = 16) | (n = 8) | (n = 8) | (n = 8) | (n = 15)* | (n = 8) | (n = 16) | (n = 8) | (n = 8) |
| Days | 154.6 | 266.8 | 642.7 | 478.0 | 1105.1 | 496.9 | 1237.0 | 902.9 | 973.4 | 2405.7 |
| 9-14 | (161.5) | (209.9) | (439.4) | (403.1) | (863.2) | (344.7) | (685.0) | (546.7) | (759.3) | (843.1) |
| Days | 163.4 | 294.9 | 780.3 | 590.7 | 848.4 | 593.3 | 1126.0 | 865.2 | 964.5 | 1718.3 |
| 23-28 | (182.1) | (173.0) | (670.5) | (281.5) | (684.0) | (437.7) | (434.5) | (463.9) | (683.5) | (889.2) |

Note:
Totals are calculated for each subject, then a daily mean for the time period is derived by dividing each total by six.
Means by treatment group are based on the daily means for each subject.
*One subject dropped out at Day 12.

Conclusion:

The results showed a significant reduction in serum bile acids and significant increase in fecal bile acids.

### Example 21

### Human Study to Test Efficacy of ASBTI in Lowering Serum Bile Acids

LUM001 has been administered to forty patients under the age of 18 years old. Table below shows the exemplary characteristics of five patients who received LUM001. The drug was administered once-a-day (QD) in the morning for fourteen days. The levels of systemic exposure of LUM001 were measured on day eight and the drug was confirmed to be minimally absorbed by in the patients. These doses are similar to those using to treat patients with Barrett's esophagus or GERD.

TABLE 13
Pharmacokinetics of LUM001 in subjects (study NB-00-02-014)

| | LUM001 | | | |
|---|---|---|---|---|
| Subject | treatment | | Dose | Average serum drug |
| Number | (mg) | Sex | μg/kg | exposure (ng/ml) |
| 0309 | 1.0 | MALE | 35.0 | 0.0 |
| 0304 | 1.0 | MALE | 24.3 | 0.0 |
| 0308 | 1.0 | MALE | 28.9 | 0.0 |
| 0410 | 2.5 | FEMALE | 42.0 | 0.0 |
| 0510 | 5.0 | MALE | 168.4 | 0.0 |

The efficacy of LUM001 was determined by measuring total serum bile acids after eight days of dosing in children and adolescents under the age of eighteen. Thirty minutes before the next drug administration, at approximately 8 am in the morning, serum bile acid levels were measured. The child had refrained from food for 12 hours prior to this sample thus providing a fasted level of serum bile acid. After breakfast, serum bile acids were measured for up to the next 4 hours (8 am to noon) and the peak serum bile acid concentration noted. LUM001 was shown to generally decrease both the fasting and post-prandial peak levels of serum bile acids (see table). In the table below the placebo patients had an average fasting serum bile acid level of 8.6 won and a post-prandial peak serum bile acid level of 11.9 μmol/L. For the LUM001 treated patients the values were 6.5 won and 9.2, respectively, representing a 24% and 23% decrease (see FIG. 7).

TABLE 14
Fasting SBA and morning post-prandial peak in subjects

| | Patients | | | | | | | | | |
|---|---|---|---|---|---|---|---|---|---|---|
| | 301 | 307 | 405 | 408 | 508 | 304 | 308 | 309 | 401 | 510 |
| Drug dose (mg) | Placebo | Placebo | Placebo | Placebo | Placebo | 1 | 1 | 1 | 2.5 | 5 |
| Fasting serum | 9.1 | 7.4 | 10.5 | 8.3 | 7.7 | 5.6 | 6.8 | 6.9 | 6.0 | 7.4 |
| bile acid (μmol/l) | | | | | | | | | | |
| Morning Post- | 11.9 | 10.7 | 13.1 | 13.4 | 10.4 | 8.4 | 9.3 | 10.0 | 6.8 | 11.3 |
| prandial peak | | | | | | | | | | |
| (μmol/l) | | | | | | | | | | |

### Example 22

### Pharmacokinetics Demonstrating Nonsystemic Absorption of LUM001

This was an open-label, one-period, single-dose study of a [¹⁴C]LUM001 oral solution. Eight healthy male subjects received an oral solution of 5 mg [¹⁴C]LUM001 (containing approximately 100 Ci per dose). Blood samples were collected at predose and for 72 hours postdose, urine samples were collected predose and for 168 hours postdose and feces were collected predose and for 216 hours postdose. Plasma, whole blood, urine and fecal samples were analyzed for total radioactivity, and selected fecal samples were analyzed for LUM001 and relevant metabolite concentrations. In addition, metabolic profiles in selected fecal samples were obtained using high performance liquid radio chromatography (HPLRC).

Pharmacokinetic Results:

Less than 1% of the radioactive dose was detected in plasma, whole blood or urine. Seventy-two percent (72%) of the total radioactive dose was detected in feces.

Characterization of the fecal analytes indicated that 94% of the fecal radioactivity was associated with unchanged free LUM001. Three fecal metabolites were identified (M1, M3 and M4). Less than 3% of the fecal radioactivity was associated with these metabolites.

TABLE 15
Mean Cumulative Percent of Total Dose of Radioactivity Present in
Plasma, Whole Blood, Feces and Urine

| | | Radioactive | Cumulative % of |
|---|---|---|---|
| | | Concentration* | Total Dose |
| Plasma | | | |
| 0.5 | hr | BLQ | — |
| 1 | hr | BLQ | — |
| 1.5 | hr | BLQ | — |
| 2 | hr | BLQ | — |
| 2.5 | hr | BLQ | — |
| 3 | hr | BLQ | — |
| 4 | hr | BLQ | — |
| 6 | hr | BLQ | — |
| 8 | hr | BLQ | — |
| 12 | hr | BLQ | — |
| 16 | hr | 0.000 | — |
| 24 | hr | BLQ | — |
| 36 | hr | 0.000 | — |
| 48 | hr | BLQ | — |
| 60 | hr | BLQ | — |
| 72 | hr | BLQ | — |
| Whole Blood | | | |
| −0.5 | hr | 0.000 | — |
| 1 | hr | 0.000 | — |
| 4 | hr | 0.000 | — |
| Feces | | | |
| 0-24 | hr | — | 30.376 |
| 24-48 | hr | — | 49.878 |
| 48-72 | hr | — | 67.630 |
| 72-96 | hr | — | 69.653 |
| 96-120 | hr | — | 71.606 |
| 120-144 | hr | — | 71.771 |
| 144-168 | hr | — | 71.815 |
| 168-192 | hr | — | 71.885 |
| 192-216 | hr | — | 71.885 |
| Toilet Paper** | | — | 72.466 |
| Urine | | | |
| 0-2 | hr | — | 0.005 |
| 2-4 | hr | — | 0.012 |
| 4-8 | hr | — | 0.028 |
| 8-12 | hr | — | 0.041 |
| 12-24 | hr | — | 0.058 |
| 24-48 | hr | — | 0.057 |
| 48-72 | hr | — | 0.062 |
| 72-96 | hr | — | 0.063 |
| 96-120 | hr | — | 0.063 |
| 120-144 | hr | — | 0.064 |
| 144-168 | hr | — | 0.066 |

| | | |
|---|---|---|
| Total (Urine and Feces) | — | 72.532 |

*ng equivalents/mL for plasma; ng equivalents/g for red blood cells. —Analysis not performed or data not provided in source document. **Cumulative recovery in toilet paper from first five fecal samples. BLQ = Below Lower Limit of Quantitation.

TABLE 16
Mean Percent of Dose in Feces and Percent of Total Fecal Radioactivity for
LUM001 and Metabolites

| | | | Percent of Fecal | |
|---|---|---|---|---|
| Analyte | Percent of Dose | | Radioactivity | |
| SD-5613 | 65.0 ± | 3.1 | 94.4 ± | 1.35 |
| M1 | NA (a) | | NA (a) | |
| M3 | 1.67 ± | 0.42 | 2.40 ± | 0.574 |

| | | |
|---|---|---|
| M4 | NA (a) | NA (a) |

(a) Not Applicable. Less than 1% of dose. M1 was identified as the N-demethylated metabolite of LUM001. M3 was identified as the monohydroxylation metabolite, with the hydroxylation occurring at the −position on the butyl chain. M4 was identified as the N-demethylated M3. The majority of the radioactivity was below the level of detection in plasma and whole blood. Small amounts of radioactivity (<1% of the dose) were detected in urine.

### Example 23

### Animal Efficacy Study on Oral Dose of LUM002 on Fecal Bile Acids in Hamsters

Test Compound:

LUM002

Animal Handling, Dosing and Sample Collection:

To study the effect of LUM002 on fecal excretion of [¹⁴C]taurocholic acid (TCA) in the Syrian hamster in vivo, [¹⁴C]TCA was administered intraperitoneally 1 hour before dosing the test compound. Each animal was dosed with 1 μCi [¹⁴C]TCA. LUM002 was dosed by oral gavage (5 mL/kg per administration) in either 5% (v/v) Solutol with 0.5% hydroxyethylcellulose (HEC) or 0.5% HEC alone. The first half of dose of test compound/vehicle was given 1 hour after [¹⁴Cl]TCA injection, followed by the second half of dose 7 hours after the first dose of test compound/vehicle. Feces were collected for 24 hours after [¹⁴Cl]TCA administration and aliquots from feces were combusted for determination of [¹⁴Cl] excretion.

Fecal Bile Acid Measurement:

The ED₂₀₀ (dose which doubles the fecal excretion of [¹⁴Cl] label vs. control from dose response curve, including 95% Confidence interval) was calculated from a dose response curve.

Results:

LUM002 significantly increased fecal excretion of bile acids. (FIG. 8; mean±SEM; n=4 nonfasted Golden Syrian hamsters per group).

### Example 24

### Pharmacokinetics Demonstrating Nonsystemic Absorption of LUM002 in Rats

To each rat either a single oral (PO; 20 mg/kg free acid) or a single intravenous (IV; 2 mg/kg free acid) dose of [¹⁴Cl]LUM002 was administered. For PO and IV administration, the test substance was prepared with the adequate amount of water to reach a solution with a concentration of approximately 4 mg/g (PO) and 1 mg/g (IV). Oral administration was a gavage using a stomach tube and IV administration was a bolus injection into the tail vein. Exact doses administered to animals were calculated according to syringe weights.

The doses of radioactivity actually administered were determined by using standards prior to and after treatment of the animals. Blood—Animals were anaesthetized (Isoflurane) for blood sampling (3 animals/time point) and then euthanized by cerebral dislocation. Blood samples (maximal volume from each animal approx. 8 mL) were withdrawn from the abdominal aorta into containers containing a small amount of lithium heparin. and samples were centrifuged for 2 minutes at 1540 g. Aliquots (100 μL) of blood were used for hematocrit determination. Animals for the control groups (male and/or female) were used as predose animals for sampling of untreated biological samples (blood and plasma or liver, myocardium, kidneys only from male control animals). Plasma—To obtain plasma, blood samples were centrifuged for 10 minutes and stored cooled before final division.

Bioanalytical analysis—50 μL of plasma samples were placed into an 1.5 mL reaction tube. 5 μL methanol was added to each sample. For protein precipitation, 100 μL of the internal standard working solution (100 ng/mL of benzyl-13C6-LUM002 in acetonitrile) and 50 μL acetonitrile were added. In case of double blank samples, pure acetonitrile was added. Tubes were sealed and mixed thoroughly for 10 seconds. The tubes were centrifuged for 5 minutes at ≧3000 g. 50 μL of the clear supernatant was transferred into an autoinjector vial and were diluted with 50 μL deionized water. Analysis of samples was performed by using LC-MSMS (injection volume 50-75 μL) with a lower limit of quantification in the assay of 1 ng/mL for LUM002.

Pharmacokinetic Results:

Less than 2% of the LUM002 dose was systemically absorbed.

TABLE 17
Pharmacokinetics of LUM002 Following Single Administration to Rats

| | | | Dose | Tₘₐₓ | Cₘₐₓ | AUC_{inf} | CL | Vₛₛ | t_{1/2} | F |
|---|---|---|---|---|---|---|---|---|---|---|
| Species | Sex | Route | (mg/kg) | (h) | (ng/mL) | (ng.h/mL) | (L/h) | (L/kg) | (h) | (%) |
| Sprague | M | IV^{b} | 2 | 0^{c} | 1036 | 0.9 | 179 | 3.890 | 10.7 | — |
| Dawley | | | | | | | | | | |
| Rat | | | | | | | | | | |
| Sprague | M | PO | 20 | —^{d} | —^{d} | — | —^{d} | — | — | <1.3^{e} |
| Dawley | | | | | | | | | | |
| Rat | | | | | | | | | | |
| Sprague | F | PO | 20 | —^{d} | —^{d} | — | —^{d} | — | — | <1.3^{e} |
| Dawley | | | | | | | | | | |
| Rat | | | | | | | | | | |

^{a}free acid;
^{b}bolus injection;
^{c}extrapolated to t = 0;
^{d}only 3 female and 1 male animal out of 72 showed exposures between 1-5 ng/mL 2 or 4 h after administration. One male animal (6 h) showed an exposure of 323 ng/mL most probably due to a contamination. All other animals showed an exposure below limit of quantitation (1 ng/mL);
^{e}for estimation of a topmost bioavailability, a topmost exposure of 1 ng/mL over 24 h (at 20 mg/kg dose) and a dose-linear exposure was assumed.

### Example 25

### Pharmacokinetics Demonstrating Nonsystemic Absorption of LUM002 in Humans

Healthy male subjects, from 18 to 45 years of age, were given a single oral solution dose (100 mg in 100 mL) of LUM002 under fasting conditions. Blood samples for the determination of LUM002 plasma concentration were collected predose and 0.5, 1, 2, 3, 4, 5, 6, 8, 10, 12, 16, 24, and 48 hours after treatment administration. Plasma was obtained by centrifugation and 150 mL processed for protein precipitation, centrifuged again to remove the precipitate and the supernatant prepared for LC-MSMS analysis. LUM002 plasma concentrations were determined using a validated liquid chromatography tandem mass spectrometry (LC-MSMS) assay with a lower limit of quantification (LLOQ) of 0.1 ng/mL. LUM002 plasma concentrations were used to determine the following pharmacokinetic parameters using standard noncompartmental techniques: Cₘₐₓ, Tₘₐₓ, AUCₗₐₛₜ, and, if applicable due to the low-absorbable characteristics of LUM002, also AUC, AUC₀₋₂₄ and t_{1/2}.

Pharmacokinetic Results:

Less than 1% of the LUM002 dose was systemically absorbed.

TABLE 18
TABLE OF INDIVIDUAL PHARMACOKINETIC PARAMETERS FOR
TREATMENT WITH LUM002 AT A DOSE OF 100 MG
Treatment = SAR548304B 100 mg
Plasma SAR548304

| | Cₘₐₓ | tₘₐₓ | tₗₐₛₜ | t_{lag} | t_{1/2z} | AUCₗₐₛₜ | AUC | AUC_{Ext} | AUC₀₋₂₄ |
|---|---|---|---|---|---|---|---|---|---|
| Subject | (ng/ml) | (hr) | (hr) | (hr) | (hr) | (ng · hr/ml) | (ng · hr/ml) | (%) | (ng · hr/ml) |
| 250001149 | 0.300 | 5.00 | 8.00 | 0.00 | 3.39 | 1.19 | NC | 39 | 1.92 |
| 250001150 | 0.163 | 5.00 | 5.00 | 4.00 | NC | 0.0815 | NC | NC | NC |
| 250001151 | 0.129 | 3.00 | 3.00 | 2.00 | NC | 0.0645 | NC | NC | NC |
| 250001154 | 0.269 | 6.00 | 6.00 | 0.00 | NC | 0.215 | NC | NC | NC |
| 250001155 | 0.286 | 0.50 | 4.00 | 0.00 | NC | 0.522 | NC | NC | NC |
| 750001156 | 0.120 | 5.00 | 5.00 | 4.00 | NC | 0.0600 | NC | NC | NC |
| N | 6 | 6 | 6 | 6 | 1 | 6 | 0 | 1 | 1 |
| Men | 0.211 | 4.08 | 5.17 | 1.67 | NC | 0.355 | NC | NC | NC |
| SD | 0.0827 | 2.01 | 1.72 | 1.97 | NC | 0.445 | NC | NC | NC |
| SE | 0.0338 | 0.82 | 0.70 | 0.80 | NC | 0.182 | NC | NC | NC |
| Min | 0.120 | 0.50 | 3.00 | 0.00 | NC | 0.0600 | NC | NC | NC |
| Median | 0.216 | 5.00 | 5.00 | 1.00 | NC | 0.148 | NC | NC | NC |
| Max | 0.300 | 6.00 | 8.00 | 4.00 | NC | 1.19 | NC. | NC | NC |
| CV % | 39.2 | 49.2 | 33.3 | 118.0 | NC | 125.1 | NC | NC | NC |
| Geometric Mean | 0.197 | 3.22 | 4.93 | NA | NC | 0.187 | NC | NC | NC |

NC = Net calculated
NA = Not Applicable:

### Example 26

### Animal Efficacy Study on Effect of LUM002 and SC-435 in ZDF Rats

Test Compound:

LUM002 and SC-435

Dosage Preparation and Administration:

This study was conducted to determine the effects of two non-absorbed ASBTi compounds in the ZDF rat model after 3 weeks of treatment. This study was conducted in accordance with U.S. FDA regulations 21 CFR Part 58 Good Laboratory Practice for Nonclinical Laboratory Studies (and all amendments, effective Jun. 20, 1979).

Animals: 80 male obese ZDF rats (Zucker Diabetic fatty (ZDF/GmiCrl-fa/fa) 8 wk. old, 280 g) Charles River (Wilmington, Mass.). Animals were single housed and fed ad libitum with Purina #5008 diet. Animals were acclimatized for 5-7 days after arrival. The study was performed in two sets: each set had 4 animals per treatment group. The 2^{nd} set of study was initiated after completion of the 1^{st}. Test compound formulation prepared every other day and stored at 4° C. Blood glucose, HbA1C, tGLP-1 and tGLP-2 tested at initiation of study and weeks 1 and 2 on non-fasted animals. Feces collected (24 hour) to evaluate the levels of fecal bile acids on the 10^{th} days after the start of the test articles administration. Bile acids in fecal extraction were measured by kits (Diazyme Inc., San Diego, Calif.). After 3rd week of treatment blood samples were collected by cardiac puncture from fasted animals for blood chemistry analysis: ALAT, ASAT, GGT, Alkaline Phosphatase and total Bile Acids. Tests were performed in contract clinical laboratory (Liver Profile SA320, Antech Diagnostics, Orange Country, Calif.).

Compounds administered twice daily by oral gavage (ngroup): Water, vehicle control (8); SAR 548304 (LUM001) 0.01 (6), 0.1 (6), 0.3 (3), 1 (7), 3 (4), 10 (8), 30 (3) mg/kg; SC-4350.1 (6), 1 (6), 10 (7) and 30 (3) mg/kg.

Key efficacy, metabolic and liver function parameters were assessed: Total 24 hr fecal and total serum bile acid concentrations; Plasma ALP, ALT, AST, BUN, creatinine; Fasting glucose and insulin concentrations, glucoseinsulin ratio, blood percent HbA1c and Oral Glucose Tolerance Test (glucose excursion, insulin, GLP-1); Plasma GLP-1, GLP-2 and FGF21 concentrations.

Male ZDF rats were administered water vehicle, SAR 548304 (0.01, 0.1, 0.3, 1, 3, 10, 30 or 100 mg/kg) or SC-435 (0.1, 1, 10 or 30 mg/kg) twice daily by oral gavage for 3 weeks. Phase 1 doses: LUM002=0.1, 1, 10, 30, 100 mg/kg; SC-435=1, 10, 30 mg/kg. Phase 2 doses: LUM002=0.01, 0.1, 1, 10 mg/kg; SC-435=0.1, 1, 10 mg/kg. Blood was collected by tail vein bleed weekly and after 3 weeks of treatment by cardiopuncture for serum tests. Data for each group is presented as the Mean Value±SEM. Statistically evaluation by t-test: *P<0.05, **P<0.01,***P<0.001 vs. vehicle group.

Results:

24 hour Fecal Bile Acids Concentrations Day 10: LUM002 and SC-435 caused statistically significant increases in 24 h total fecal BA concentration (up to 4-fold compared with vehicle-treated rats) (FIGS. 9A and 9B).

Plasma Total Serum Bile Acids Concentrations—Week 3:

Both LUM002 and SC-435 caused a statistically significant reductions in SBA (FIGS. 10A and 10B).

Liver Function: Plasma Alkaline Phosphatase—Week 3:

Both LUM002 and SC-435 caused significant reductions in ALP (FIGS. 11A and 11B).

Liver Function: Plasma Aspartate Aminotransferase—Week 3:

LUM002 caused a statistically significant reduction in ASAT (FIGS. 12A and 12B).

Liver Function: Plasma Alanine Aminotransferase—Week 3:

Neither LUM002 nor SC-435 caused statistically significant reductions in plasma ALAT (FIG. 13).

Plasma Triglycerides—Week 3:

LUM002 and SC435 caused statistically significant elevations in fasting plasma triglycerides although there was no dose-related response (likely due to animal variability in response) (FIG. 14).

Baseline-Corrected Percent Hemoglobin A1c (HbA1c):

Both LUM002 and SC-435 caused significant dose-dependent reductions in baseline-corrected HbA1c (FIGS. 15A and 15B; *p<0.05, **p<0.01 and ***p<0.001 vs vehicle group).

GLP-2 in Plasma of Non-Fasted ZDF Rats 2 Weeks Treatment:

Both LUM002 and SC-435 caused significant elevations in GLP2 at higher doses (FIGS. 16A and 16B).

Exocrine Pancreas Function: Plasma Lipase—Week 3:

LUM002 and SC-435 caused statistically significant reductions in plasma lipase at higher doses (FIG. 17).

Exocrine Pancreas Function: Plasma Amylase—Week 3:

Neither LUM002 nor SC-435 caused a statistically significant changes in plasma amylase (FIGS. 18A and 18B).

### Example 27

### Clinical Trial to Test Efficacy of ASBTI in Treatment and/or Alleviation of Symptoms of Barrett's Esophagus

This study will determine efficacy of ASBTI treatment in patients afflicted with Barrett's esophagus.

Subjects 18 years of age or older, clinically diagnosed with Barrett's esophagus will be enrolled. Subjects may be diagnosed by symptoms such as intestinal metaplasia, esophageal damage or necrosis, hematemesis, heartburn, regurgitation, dysphagia, odynophagia, nausea, weight loss, increased salivation, chest pain, reflux esophagitis, esophageal strictures, laryngitis, asthma, sinusitis, pharyngitis, globus pharingeus, globus hystericus, enamel erosion, and dentine hypersensitivity.

Subjects who have life threatening renal disease, cardiovascular disease, or congenital anomalies will be excluded.

Subjects will be administered a daily oral dose of LUM001 formulated for release in the distal ileum. Alternatively, any of the following compounds can be the subject of the clinical trial: LUM002; SC-435; 264W94; 100B; SA HMR1741; 1,1-Dioxo-3,3-dibutyl-5-phenyl-7-methylthio-8-(N-[(R)-α-[N-(2-sulphoethyl)carbamoyl]-4-hydroxybenzyl]carbamoylmethoxy)-2,3,4,5-tetrahydro-1,2,5-benzothiadiazepine; 1,1-Dioxo-3,3-dibutyl-5-phenyl-7-methylthio-8-(N—[(R)-α-[N—((S)-1-carboxy-2-(R)-hydroxypropyl)carbamoyl]-4-hydroxybenzyl]carbamoylmethoxy)-2,3,4,5-tetrahydro-1,2,5-benzothiadiazepine; 1,1-Dioxo-3,3-dibutyl-5-phenyl-7-methylthio-8-(N—[(R)-α-[N—((S)-1-carboxy-2-methylpropyl)carbamoyl]-4-hydroxybenzyl]carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N(S)-1-carboxypropyl)carbamoyl]-4-hydroxybenzyl}carbamoylmethoxy)-2,3,4,5-tetrahydro-1,2,5-benzothiadiazepine; or 1,1-dioxo-3,3-dibutyl-5-phenyl-7-methylthio-8-[N—((R)-α-carboxy-4-hydroxybenzyl)carbamoylmethoxy]-2,3,4,5-tetrahydro-1,2,5-benzothiadiazepine.

The primary endpoint is the proportion of subjects showing resolution or improvement of baseline signs and symptoms, e.g., serum levels of bile acidssalts and/or GLP-2.

### Example 28

### Clinical Trial to Test Efficacy of ASBTI in Treatment and/or Alleviation of Symptoms of GERD

This study will determine efficacy of an ASBTI for treatment in patients afflicted with GERD.

Subjects 18 years of age or older, clinically diagnosed with GERD will be enrolled. Subjects may be diagnosed by symptoms such as hematemesis, heartburn, regurgitation, dysphagia, odynophagia, nausea, weight loss, increased salivation, chest pain, reflux esophagitis, esophageal strictures, laryngitis, asthma, sinusitis, pharyngitis, globus pharingeus, globus hystericus, enamel erosion, and dentine hypersensitivity are eligible for enrollment.

Subjects will be administered a daily oral dose of LUM001 formulated for release in the distal ileum. Alternatively, any of the following compounds can be the subject of the clinical trial: LUM002; SC-435; 264W94; SA HMR1741; 1,1-Dioxo-3,3-dibutyl-5-phenyl-7-methylthio-8-(N—[(R)-α-1N-(2-sulphoethyl)carbamoyl]-4-hydroxybenzyl]carbamoylmethoxy)-2,3,4,5-tetrahydro-1,2,5-benzothiadiazepine; 1,1-Dioxo-3,3-dibutyl-5-phenyl-7-methylthio-8-(N—[(R)-α-[N—((S)-1-carboxy-2-(R)-hydroxypropyl)carbamoyl]-4-hydroxybenzyl]carbamoylmethoxy)-2,3,4,5-tetrahydro-1,2,5-benzothiadiazepine; 1,1-Dioxo-3,3-dibutyl-5-phenyl-7-methylthio-8-(N—[(R)-α-[N—((S)-1-carboxy-2-methylpropyl)carbamoyl]-4-hydroxybenzyl]carbamoylmethoxy)-2,3,4,5-tetrahydro-1,2,5-benzothiadiazepine; 1,1-dioxo-3,3-dibutyl-5-phenyl-7-methylthio-8-(N-{(R)-α-[N—((S)-1-carboxypropyl)carbamoyl]-4-hydroxybenzyl}carbamoylmethoxy)-2,3,4,5-tetrahydro-1,2,5-benzothiadiazepine; or 1,1-dioxo-3,3-dibutyl-5-phenyl-7-methylthio-8-[N—((R)-α-carboxy-4-hydroxybenzyl)carbamoylmethoxy]-2,3,4,5-tetrahydro-1,2,5-benzothiadiazepine.

Stage 1 will be a 4 week dose escalation study to determine patient minimum tolerated dose. Dose 1: 14 μg/kg/day for 7 days; dose 2: 35 μg/kg/day for 7 days; dose 3; 70 μg/kg/day for 7 days; dose 4: 140 μg/kg/day for 7 days.

Stage 2 will be a double-blind placebo controlled cross-over study. Subjects will be randomized to maximum tolerated dose or placebo for 8 weeks, followed by a 2 week drug holiday, and cross-over to receive the alternative regimen for 8 week.

The primary endpoint is the proportion of subjects showing resolution or improvement of baseline signs and symptoms, e.g., serum levels of bile acidssalts, GLP-2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating or ameliorating Barrett's esophagus or gastro-esophageal reflux disease (GERD) in an individual comprising non-systemically administering to the individual a therapeutically effective amount of an Apical Sodium-dependent Bile Acid Transporter Inhibitor (ASBTI) or a pharmaceutically acceptable salt or solvate thereof.

2. The method of claim 1, wherein the method decreases gastroesophageal reflux of bile acid.

3. The method of claim 1, wherein the method decreases the risk of esophageal adenocarcinoma.

4. The method of claim 1, wherein the method comprises increasing at least 10% of GLP-2 levels in the individual.

5. The method of claim 1, wherein the method comprises decreasing at least 20% of serum bile acid or hepatic bile acid levels in the individual.

6. The method of claim 1, wherein the method comprises increasing at least 20% of fecal bile acid levels in the individual.

7. The method of claim 1, wherein less than 10% of the ASBTI is systemically absorbed.

8. The method of claim 1, wherein the ASBTI is a compound of Formula II: [chemical structure]
wherein:
q is an integer from 1 to 4;
n is an integer from 0 to 2;
R¹ and R² are independently selected from the group consisting of H, alkyl, alkenyl, alkynyl, haloalkyl, alkylaryl, arylalkyl, alkoxy, alkoxyalkyl, dialkylamino, alkylthio, (polyalkyl)aryl, and cycloalkyl,
wherein alkyl, alkenyl, alkynyl, haloalkyl, alkylaryl, arylalkyl, alkoxy, alkoxyalkyl, dialkylamino, alkylthio, (polyalkyl)aryl, and cycloalkyl optionally are substituted with one or more substituents selected from the group consisting of OR⁹, NR⁹R¹⁰, N⁺R⁹R¹⁰R^{w}A⁻, SR⁹, S⁺R⁹R¹⁰A⁻, P⁺R⁹R¹⁰R¹¹K, S(O)R⁹, SO₂R⁹, SO₃R⁹, CO₂R⁹, CN, halogen, oxo, and CONR⁹R¹⁰,
wherein alkyl, alkenyl, alkynyl, alkylaryl, alkoxy, alkoxyalkyl, (polyalkyl)aryl, and cycloalkyl optionally have one or more carbons replaced by O, NR⁹, N⁺R⁹R¹⁰A⁻, S, SO, SO₂, S⁺R⁹A⁻, P⁺R⁹R¹⁰A⁻, or phenylene,
wherein R⁹, R¹⁰, and R^{w} are independently selected from the group consisting of H, alkyl, alkenyl, alkynyl, cycloalkyl, aryl, acyl, heterocycle, ammoniumalkyl, arylalkyl, and alkylammoniumalkyl; or
R¹ and R² taken together with the carbon to which they are attached form C₃-C₁₀ cycloalkyl;
R³ and R⁴ are independently selected from the group consisting of H, alkyl, alkenyl, alkynyl, acyloxy, aryl, heterocycle, OR⁹, NR⁹R¹⁰, SR⁹, S(O)R⁹, SO₂R⁹, and SO₃R⁹, wherein R⁹ and R¹⁰ are as defined above; or
R³ and R⁴ together ═O, ═NOR¹¹, ═S, ═NNR¹¹R¹², ═NR⁹, or ═CR¹¹R¹²,
wherein R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ and R⁶ are independently selected from the group consisting of H, alkyl, alkenyl, alkynyl, aryl, cycloalkyl, heterocycle, quaternary heterocycle, quaternary heteroaryl, OR⁹, SR⁹, S(O)R⁹, SO₂R⁹, SO₃R⁹, and -L_{z}-K_{z};
wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, R¹⁵, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
wherein:
A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quaternary heteroaryl, quaternary heteroarylalkyl, and G-T-V—W,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR, P⁺R⁹R¹⁰A⁻, P(O)R⁹, phenylene, carbohydrate, C₂-C₇ polyol, amino acid, peptide, or polypeptide, and
G, T and V are each independently a bond, —O—, —S—, —N(H)—, substituted or unsubstituted alkyl, —O-alkyl, —N(H)-alkyl, —C(O)N(H)—, —N(H)C(O)—, —N(H)C(O)N(H)—, substituted or unsubstituted alkenyl, substituted or unsubstituted alkynyl, substituted or unsubstituted aryl, substituted or unsubstituted arylalkyl, substituted or unsubstituted alkenylalkyl, alkynylalkyl, substituted or unsubstituted heteroalkyl, substituted or unsubstituted heterocycle, substituted or unsubstituted carboxyalkyl, substituted or unsubstituted carboalkoxyalkyl, or substituted or unsubstituted cycloalkyl, and
W is quaternary heterocycle, quaternary heteroaryl, quaternary heteroarylalkyl, N⁺R¹¹R¹²A⁻, P⁺R⁹R¹⁰R¹¹A⁻, OS(O)₂OM, or SR⁹R¹⁰A⁻, and R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R⁹R¹¹R¹²A⁻, SR⁹, S(O) R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cycloalkyl, aryl, acyl, heterocycle, ammoniumalkyl, alkylammoniumalkyl, and arylalkyl; and
R⁷ and R⁸ are independently selected from the group consisting of hydrogen and alkyl; and
one or more R^{x} are independently selected from the group consisting of H, alkyl, alkenyl, alkynyl, polyalkyl, acyloxy, aryl, arylalkyl, halogen, haloalkyl, cycloalkyl, heterocycle, heteroaryl, polyether, quaternary heterocycle, quaternary heteroaryl, OR¹³, NR¹³R¹⁴, SR¹³, S(O)R¹³, S(O)₂R¹³, SO₃R¹³S⁺R¹³R¹⁴A⁻, NR¹³OR¹⁴, NR¹³NR¹⁴R¹⁵, NO₂, CO₂R¹³, CN, OM, SO₂OM, SO₂NR¹³R¹⁴, NR¹⁴C(O)R¹³, C(O)NR¹³R¹⁴, NR¹⁴C(O)R¹³, C(O)OM, COR¹³, OR¹⁸, S(O)ₙNR¹⁸, NR¹³R¹⁸, NR¹⁸R¹⁴, N⁺R⁹R¹¹R¹²A⁻, P⁺R⁹R¹¹R¹²A⁻, amino acid, peptide, polypeptide, and carbohydrate;
wherein alkyl, alkenyl, alkynyl, cycloalkyl, aryl, polyalkyl, heterocycle, acyloxy, arylalkyl, haloalkyl, polyether, quatemary heterocycle, and quaternary heteroaryl can be further substituted with OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹¹R¹²A⁻, S⁺R⁹R¹⁰A⁻, or C(O)M,
wherein W is O or NH, R³¹ is selected from
wherein R¹⁸ is selected from the group consisting of acyl, arylalkoxycarbonyl, arylalkyl, heterocycle, heteroaryl, alkyl,
wherein acyl, arylalkoxycarbonyl, arylalkyl, heterocycle, heteroaryl, alkyl, quaternary heterocycle, and quaternary heteroaryl optionally are substituted with one or more substituents selected from the group consisting of OR⁹, NR⁹R¹⁰, N⁺R⁹R¹¹R¹²A⁻, SR⁹, S(O)R⁹, SO₂R⁹, SO₃R⁹, oxo, CO₃R⁹, CN, halogen, CONR⁹R¹⁰, SO₃R⁹, SO₂OM, SO₂NR⁹R¹⁰, PO(OR¹⁶)OR¹⁷, and C(O)OM,
wherein in R^{x}, one or more carbons are optionally replaced by O, NR¹³, N⁺R¹³R¹⁴A⁻, S, SO, SO₂, S⁺R¹³A⁻, PR¹³, P(O)R¹³, P⁺R¹³R¹⁴, A⁻, phenylene, amino acid, peptide, polypeptide, carbohydrate, polyether, or polyalkyl,
wherein in said polyalkyl, phenylene, amino acid, peptide, polypeptide, and carbohydrate, one or carbons are optionally replaced by O, NR⁹R⁹R¹⁰A⁻, S, SO, SO₂, S⁺R⁹A⁻, PR⁹P⁺R⁹R¹⁰A⁻, or P(O)R⁹;
wherein quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
provided that both R⁵ and R⁶ cannot be hydrogen or SH;
provided that when R⁵ or R⁶ is phenyl, only one of R¹ or R² is H;
provided that when q=1 and R^{x} is styryl, anilido, or anilinocarbonyl, only one of R⁵ or R⁶ is alkyl;
or a pharmaceutically acceptable salt or solvate thereof.

9. The method of claim 8, wherein:
q is 1;
n is 2;
R^{x} is N(CH₃)₂;
R⁷ and R⁸ are independently H;
R¹ and R² is alkyl;
R³ is H, and R⁴ is OH;
R⁵ is H, and R⁶ is selected from the group consisting of alkyl, alkenyl, alkynyl, aryl, cycloalkyl, heterocycle, quaternary heterocycle, quaternary heteroaryl, OR⁹, SR⁹, S(O)R⁹, SO₂R⁹, SO₃R⁹, and -L_{z}-K_{z};
wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, R¹⁵, OR¹³, OR¹³R¹⁴, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, CR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻,
wherein A⁻ is a pharmaceutically acceptable anion and M is a pharmaceutically acceptable cation, said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷) OR⁸ and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, aryl, arylalkyl, cycloalkyl, heterocycle, heteroaryl, quaternary heterocycle, quatemary heteroaryl, quatemary heteroarylalkyl, and -G-T-V—W,
wherein alkyl, alkenyl, alkynyl, arylalkyl, heterocycle, and polyalkyl optionally have one or more carbons replaced by O, NR⁹, N′R⁹R¹⁰A⁻, S, SO, SO₂, S⁺R⁹A⁻, PR, P⁺R⁹R¹⁰A⁻, P(O)R⁹, phenylene, carbohydrate, C₂-C₇ polyol, amino acid, peptide, or polypeptide, and
G, T and V are each independently a bond, —O—, —S—, —N(H)—, substituted or unsubstituted alkyl, —O-alkyl, —N(H)-alkyl, —C(O)N(H)—, —N(H)C(O)—, —N(H)C(O)N(H)—, substituted or unsubstituted alkenyl, substituted or unsubstituted alkynyl, substituted or unsubstituted aryl, substituted or unsubstituted arylalkyl, substituted or unsubstituted alkenylalkyl, alkynylalkyl, substituted or unsubstituted heteroalkyl, substituted or unsubstituted heterocycle, substituted or unsubstituted carboxyalkyl, substituted or unsubstituted carboalkoxyalkyl, or substituted or unsubstituted cycloalkyl, and
W i s quaternary heterocycle, quaternary heteroaryl, quaternary heteroarylalkyl, N⁺R¹¹R¹²A⁻, P⁺R⁹R¹⁰R¹¹A⁻, OS(O)₂OM, or S⁺R¹⁰A⁻, and
R⁹ and R¹⁰ are independently selected from the group consisting of H, alkyl, alkenyl, alkynyl, cycloalkyl, aryl, acyl, heterocycle, ammoniumalkyl, arylalkyl, and alkylammoniumalkyl;
R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR⁹R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R¹³, R¹⁴ and R¹⁵ are optionally substituted with one or more groups selected from the group consisting of sulfoalkyl, quaternary heterocycle, quaternary heteroaryl, OR⁹, NR⁹R¹⁰, N⁺R¹¹R¹²A⁻, SR⁹, S(O) R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R¹⁰R¹¹A⁻, S⁺R¹⁰A⁻, and C(O)OM,
wherein R¹⁶ and R¹⁷ are independently selected from the substituents constituting R⁹ and M; or
R¹⁴ and R¹⁵, together with the nitrogen atom to which they are attached, form a cyclic ring; and is selected from the group consisting of alkyl, alkenyl, alkynyl, cyclo alkyl, aryl, acyl, heterocycle, ammoniumalkyl, alkylammoniumalkyl, and arylalkyl.

10. The method of claim 8, wherein the compound of Formula II is [chemical structure]
or a pharmaceutically acceptable solvate or alternative salt thereof.

11. The method of claim 8, wherein the compound of Formula II is [chemical structure]
or a pharmaceutically acceptable solvate or alternative salt thereof.

12. The method of claim 8, wherein the compound of Formula II is [chemical structure]
or a pharmaceutically acceptable solvate or salt thereof.

13. The method of claim 8, wherein the compound of Formula II is [chemical structure]
potassium((2R,3R,4S,5R,6R)-4-benzyloxy-6-{3-[3-((3 S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydropyran-2-ylmethyl)sulphate ethanolate hydrate; or an alternative pharmaceutically acceptable salt or solvate thereof.

14. The method of claim 1, wherein the ASBTI is a compound of Formula I: [chemical structure]
wherein:
R¹ is a straight chained C₁₋₆ alkyl group;
R² is a straight chained C₁₋₆ alkyl group;
R³ is hydrogen or a group OR¹¹ in which R¹¹ is hydrogen, optionally substituted C₁₋₆ alkyl or a C₁₋₆ alkylcarbonyl group;
R⁴ is pyridyl or optionally substituted phenyl or -L_{z}-K_{z}; wherein z is 1, 2 or 3; each L is independently a substituted or unsubstituted alkyl, a substituted or unsubstituted heteroalkyl, a substituted or unsubstituted alkoxy, a substituted or unsubstituted aminoalkyl group, a substituted or unsubstituted aryl, a substituted or unsubstituted heteroaryl, a substituted or unsubstituted cycloalkyl, or a substituted or unsubstituted heterocycloalkyl; each K is a moiety that prevents systemic absorption;
R⁵, R⁶, R⁷ and R⁸ are the same or different and each is selected from hydrogen, halogen, cyano, R⁵-acetylide, OR¹⁵, optionally substituted C₁₋₆ alkyl, COR¹⁵, CH(OH)R¹⁵, S(O)ₙR¹⁵, P(O)(OR¹⁵)₂, OCOR¹⁵, OCF3, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)ₙR¹⁵, O(CH₂)ₚSO₃R¹⁵, O(CH₂)ₚNR¹²R¹³, O(CH₂)ₚN⁺R¹²R¹³R¹⁴ and W—R³¹, wherein W is O or NH, and R³¹ is selected from [chemical structure]
wherein p is an integer from 1-4, n is an integer from 0-3 and, R¹², R¹³, R¹⁴ and R¹⁵ are independently selected from hydrogen and optionally substituted C₁₋₆ alkyl; or
R⁶ and R⁷ are linked to form a group [chemical structure]
wherein R¹² and R¹³ are as hereinbefore defined and m is 1 or 2; and
R⁹ and R¹⁰ are the same or different and each is selected from hydrogen or C₁₋₆ alkyl;
or pharmaceutically acceptable salt, solvate, or physiologically functional derivative thereof.

15. The method of claim 14, wherein the compound of Formula I is [chemical structure]
or a pharmaceutically acceptable solvate or salt thereof.

16. The method of claim 1, wherein the ASBTI is a compound of Formula III: [chemical structure]
wherein:
each R¹, R² is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R¹ and R² together with the nitrogen to which they are attached form a 3-8-membered ring that is optionally substituted with R⁸;
each R³, R⁴ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
R⁵ is H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl,
each R⁶, R⁷ is independently H, hydroxy, alkyl, alkoxy, —C(═X)YR⁸, —YC(═X)R⁸, substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K; or R⁶ and R⁷ taken together form a bond;
each X is independently NH, S, or O;
each Y is independently NH, S, or O;
R⁸ is substituted or unsubstituted alkyl, substituted or unsubstituted heteroalkyl, substituted or unsubstituted aryl, substituted or unsubstituted alkyl-aryl, substituted or unsubstituted cycloalkyl, substituted or unsubstituted alkyl-cycloalkyl, substituted or unsubstituted heteroaryl, substituted or unsubstituted alkyl-heteroaryl, substituted or unsubstituted heterocycloalkyl, substituted or unsubstituted alkyl-heterocycloalkyl, or -L-K;
L is Aₙ, wherein
each A is independently NR¹, S(O)ₘ, O, C(═X)Y, Y(C═X), substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl; wherein each m is independently 0-2;
n is 0-7;
K is a moiety that prevents systemic absorption;
provided that at least one of R¹, R², R³ or R⁴ is -L-K;
or a pharmaceutically acceptable salt or solvate thereof.

17. The method of claim 1, wherein the ASBTI is a compound of Formula IV: [chemical structure]
wherein
R¹ is a straight chain C₁₋₆ alkyl group;
R² is a straight chain C₁₋₆ alkyl group;
R³ is hydrogen or a group OR¹¹ in which R¹¹ is hydrogen, optionally substituted C₁₋₆ alkyl or a C₁₋₆ alkylcarbonyl group;
R⁴ is pyridyl or an optionally substituted phenyl;
R⁵, R⁶ and R⁸ are the same or different and each is selected from:
hydrogen, halogen, cyano, R¹⁵-acetylide, OR¹⁵, optionally substituted C₁₋₆ alkyl, COR¹⁵, CH(OH)R¹⁵, S(O)R¹⁵, P(O)(OR¹⁵)₂, OCOR¹⁵, OCF₃, OCN, SCN, NHCN, CH₂OR¹⁵, CHO, (CH₂)ₚCN, CONR¹²R¹³, (CH₂)ₚCO₂R¹⁵, (CH₂)ₚNR¹²R¹³, CO₂R¹⁵, NHCOCF₃, NHSO₂R¹⁵, OCH₂OR¹⁵, OCH═CHR¹⁵, O(CH₂CH₂O)₁R¹⁵, O(CH₂)ₚSO₃R¹⁵, O(CH₂)ₚNR¹²R¹³ and O(CH₂)ₚN⁺R¹²R¹³R¹⁴ wherein
p is an integer from 1-4,
n is an integer from 0-3 and
R¹², R¹³, R¹⁴ and R¹⁵ are independently selected from hydrogen and optionally substituted C₁₋₆ alkyl;
R⁷ is a group of the formula [chemical structure]
wherein the hydroxyl groups may be substituted by acetyl, benzyl, or —(C₁-C₆)-alkyl-R¹⁷,
wherein the alkyl group may be substituted with one or more hydroxyl groups;
R¹⁶ is —COOH, —CH₂—OH, —CH₂—O-Acetyl, —COOMe or —COOEt;
R¹⁷ is H, —OH, —NH₂, —COOH or COOR¹⁸;
R¹⁸ is (C₁-C₄)-alkyl or —NH—(C₁-C₄)-alkyl;
X is —NH— or —O—; and
R⁹ and R¹⁰ are the same or different and each is hydrogen or C₁-C₆ alkyl; or a pharmaceutically acceptable solvate or salt thereof.

18. The method of claim 1, wherein the ASBTI is a compound of Formula V: [chemical structure]
wherein:
R^{v} is selected from hydrogen or C₁₋₆alkyl;
One of R¹ and R² are selected from hydrogen or C₁₋₆alkyl and the other is selected from C₁₋₆ alkyl;
R^{x} and R^{y} are independently selected from hydrogen, hydroxy, amino, mercapto, C₁₋₆ alkyl, C₁₋₆alkoxy, N—(C₁₋₆ alkyl)amino, N,N—(C₁₋₆ alkyl)₂amino, C₁₋₆ alkylS(O)ₐ wherein a is 0 to 2;
R^{z} is selected from halo, nitro, cyano, hydroxy, amino, carboxy, carbamoyl, mercapto, sulphamoyl, C₁₋₆alkyl, C₂₋₆ alkenyl, C₂₋₆ alkynyl, C₁₋₆alkoxy, C₁₋₆alkanoyl, C₁₋₆alkanoyloxy, N (C₁₋₆alkyl)amino, N,N—(C₁₋₆alkyl)₂amino, C₁₋₆alkanoylamino, N—(C₁₋₆alkyl)carbamoyl, N,N (C₁₋₆alkyl)₂carbamoyl, C₁₋₆alkylS(O)ₐ wherein a is 0 to 2, C₁₋₆alkoxycarbonyl, N (C₁₋₆-alkyl)sulphamoyl and N,N—(C₁₋₆ alkyl)₂sulphamoyl;
n is 0-5;
one of R⁴ and R⁵ is a group of formula (VA): [chemical structure]
R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁-6 alkyl)₂ amino, C₁₋₆ alkanoylamino, N—(C₁₋₆ alkyl)carbamoyl, N,N—(C₁₋₆ alkyl)₂ carbamoyl, C₁₋₆ alkylS(O)ₐ wherein a is 0 to 2, C₁₋₆alkoxycarbonyl, N—(C₁₋₆alkyl)sulphamoyl and N,N—(C₁₋₆ alkyl)₂sulphamoyl;
wherein R³ and R⁶ and the other of R⁴ and R⁵ may be optionally substituted on carbon by one or more R¹⁷;
X is —O—, —N(R^{a})—, —S(O)_{b}— or —CH(R^{a})—;
wherein R^{a} is hydrogen or C₁₋₆alkyl and b is 0-2;
Ring A is aryl or heteroaryl;
wherein Ring A is optionally substituted on carbon by one or more substituents selected from R¹⁸;
R⁷ is hydrogen, C₁₋₆alkyl, carbocyclyl or heterocyclyl;
wherein R⁷ is optionally substituted on carbon by one or more substituents selected from R¹⁹; and wherein if said heterocyclyl contains an —NH— group, that nitrogen may be optionally substituted by a group selected from R²⁰;
R⁸ is hydrogen or C₁₋₆-alkyl;
R⁹ is hydrogen or C₁₋₆alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkynyl, C₂₋₁₀alkynyl, C₁₋₁₀alkoxy, C₁₋₁₀alkanoyl, C₁₋₁₀ alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂ carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀ alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹—(C₁₋₁₀alkylene)_{q}- or heterocyclyl-(C₁₋₁₀alkylene)ᵣ-R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (VB): [chemical structure]
wherein:
R¹¹ is hydrogen or C₁₋₆-alkyl;
R¹² and R¹³ are independently selected from hydrogen, halo, carbamoyl, sulphamoyl, C₁₋₁₀alkyl, C₂₋₁₀alkynyl, C₂₋₁₀alkynyl, C₁₋₁₀alkanoyl, N—(C₁₋₁₀alkyl)carbamoyl, N,N—(C₁₋₁₀ alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;
R¹⁴ is selected from hydrogen, halo, carbamoyl, sulphamoyl, hydroxyaminocarbonyl, C₁₋₁₀alkyl, C₂₋₁₀alkenyl, C₂₋₁₀alkynyl, C₁₋₁₀alkanoyl, N—(C₁₋₁₀alkyl)carbamoyl, N,N—(C₁₋₁₀ alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VC): [chemical structure]
R¹⁵ is hydrogen or C₁₋₆alkyl; and R¹⁶ is hydrogen or C₁₋₆alkyl; wherein R¹⁶ may be optionally substituted on carbon by one or more groups selected from R³¹;
or R¹⁵ and R¹⁶ together with the nitrogen to which they are attached form a heterocyclyl; wherein said heterocyclyl may be optionally substituted on carbon by one or more R³⁷; and wherein if said heterocyclyl contains an —NH— group, that nitrogen may be optionally substituted by a group selected from R³⁸;
m is 1-3; wherein the values of R⁷ may be the same or different;
R¹⁷, R¹⁸, R¹⁹, R²³, R²⁵, R²⁹, R³¹ and R³⁷ are independently selected from halo, nitro, cyano, hydroxy, amino, carbamoyl, mercapto, sulphamoyl, hydroxyaminocarbonyl, C₁₋₁₀alkyl, C₂₋₁₀ alkenyl, C₂₋₁₀alkynyl, C₁₋₁₀alkoxy, C₁₋₁₀alkanoyl, C₁₋₁₀alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R¹⁷, R¹⁸, R¹⁹, R²³, R²⁵, R²⁹, R³¹ and R³⁷ may be independently optionally substituted on carbon by one or more R³⁴; and wherein if said heterocyclyl contains an —NH— group, that nitrogen may be optionally substituted by a group selected from R³⁵;
R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;
p, q, r and s are independently selected from 0-2;
R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;
R²⁰, R²⁴, R²⁶, R³⁰, R³⁵ and R³⁸ are independently selected from C₁₋₆alkyl, C₁₋₆alkanoyl, C₁₋₆ alkylsulphonyl, C₁₋₆ alkoxycarbonyl, carbamoyl, N—(C₁₋₆ alkyl)carbamoyl, N,N—(C₁₋₆-alkyl)carbamoyl, benzyl, benzyloxycarbonyl, benzoyl and phenylsulphonyl; and
wherein a “heteroaryl” is a totally unsaturated, mono or bicyclic ring containing 3-12 atoms of which at least one atom is chosen from nitrogen, sulphur and oxygen, which heteroaryl may, unless otherwise specified, be carbon or nitrogen linked;
wherein a “heterocyclyl” is a saturated, partially saturated or unsaturated, mono or bicyclic ring containing 3-12 atoms of which at least one atom is chosen from nitrogen, sulphur and oxygen, which heterocyclyl may, unless otherwise specified, be carbon or nitrogen linked, wherein a —CH₂— group can optionally be replaced by a —C(O)— group, and a ring sulphur atom may be optionally oxidized to form an S-oxide; and
wherein a “carbocyclyl” is a saturated, partially saturated or unsaturated, mono or bicyclic carbon ring that contains 3-12 atoms; wherein a —CH₂— group can optionally be replaced by a —C(O) group;
or a pharmaceutically acceptable salt, solvate, or in vivo hydrolysable ester or amide formed on an available carboxy or hydroxy group thereof.

19. The method of claim 18, wherein the compound of Formula V is
1,1-dioxo-3,3-dibutyl-5-phenyl-7-methylthio-8-(N-{(R)-α-[N—((R)-1-carboxy-2-methylthio-ethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxybutyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxyethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carb oxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((R)-1-carboxy-2-methylthio ethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carb oxy-2-methylpropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-[N-{(R)-α-carboxy4-hydroxybenzyl}carbamoylmethoxy]-2,3,4,5-tetrahydro-1,2,5-benzothiadiazepine; or 1,1-dioxo-3,3-dibutyl-5-phenyl-7-methylthio-8-(N-{(R)-α-[N-(carboxymethyl)carbamoyl]benzyl}carbamoylmethoxy)-2,3,4,5-tetrahydro-1,2,5-benzothiadiazepine;
or a pharmaceutically acceptable salt or solvate thereof.

20. The method of claim 1, wherein the ASBTI is a compound of Formula VI: [chemical structure]
wherein:
R^{v} and R^{w} are independently selected from hydrogen or C₁₋₆alkyl;
one of R¹ and R² is selected from hydrogen or C₁₋₆alkyl and the other is selected from C₁₋₆alkyl;
R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;
R^{z} is selected from halo, nitro, cyano, hydroxy, amino, carboxy, carbamoyl, mercapto, sulphamoyl, C₁₋₆alkyl, C₂₋₆ alkenyl, C₂₋₆ alkynyl, C₁₋₆alkoxy, C₁₋₆alkanoyl, C₁₋₆alkanoyloxy, N (C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆ alkyl)sulphamoyl and N,N—(C₁₋₆alkyl)₂sulphamoyl;
n is 0-5;
one of R⁴ and R⁵ is a group of formula (VIA): [chemical structure]
R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₁₀alkoxy, C₁₋₆ alkanoyl, C₁₋₆alkanoyloxy, N—(C₁₋₆ alkyl)amino, N,N (C₁₋₆ alkyl)₂amino, C₁₋₆ alkanoylamino, N—(C₁₋₆alkyl)carbamoyl, N,N—(C₁₋₆alkyl)₂carbamoyl, C₁₋₆ alkylS(O)ₐ wherein a is 0 to 2, C₁₋₆alkoxycarbonyl, N—(C₁₋₆alkyl)sulphamoyl and N,N—(C₁₋₆ alkyl)₂sulphamoyl; wherein R³ and R⁶ and the other of R⁴ and R⁵ may be optionally substituted on carbon by one or more R¹⁷;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted on carbon by one or more substituents selected from R¹⁸;
R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an NH group, that nitrogen may be optionally substituted by a group selected from R²⁰;
R⁸ is hydrogen or C₁₋₆alkyl;
R⁹ is hydrogen or C₁₋₆alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀ alkanoyloxy, N—(C₁₋₁₀alkyl)amino, N,N—(C₁₋₁₀alkyl)₂amino, N,N,N—(C₁₋₁₀alkyl)₃ammonio, C₁₋₁₀alkanoylamino, N—(C₁₋₁₀alkyl)carbamoyl, N,N—(C₁₋₁₀alkyl)₂ carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀ alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹—(C₁₋₁₀alkylene)_{q}- or heterocyclyl-(C₁₋₁₀alkylene)ᵣ-R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (VIB): [chemical structure]
wherein:
R¹¹ is hydrogen or C₁₋₆alkyl;
R¹² and R¹³ are independently selected from hydrogen, halo, nitro, cyano, hydroxy, amino, carbamoyl, mercapto, sulphamoyl, C₁₋₁₀alkyl, C₂₋₁₀alkenyl, C₂₋₁₀alkynyl, C₁₋₁₀alkoxy, C₁₋₁₀ alkanoyl, C₁₋₁₀alkanoyloxy, N—(C₁₋₁₀alkyl)amino, N,N—(C₁₋₁₀alkyl)₂amino, C₁₋₁₀ alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an NH group, that nitrogen may be optionally substituted by a group selected from R²⁶;
R¹⁴ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀ alkanoyl, C₁₋₁₀alkanoyloxy, N—(C₁₋₁₀alkyl)amino, N,N—(C₁₋₁₀alkyl)₂amino, N,N,N—(C₁₋₁₀ alkyl)₃ammonio, C₁₋₁₀alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀alkyl)sulphamoylamino, N,N—(C₁₋₁₀alkyl)₂sulphamoylamino, C₁₋₁₀ alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀-alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (VIC): [chemical structure]
R¹⁵ is hydrogen or C₁₋₆ alkyl;
R¹⁶ is hydrogen or C₁₋₆alkyl; wherein R¹⁶ may be optionally substituted on carbon by one or more groups selected from R³¹;
n is 1-3; wherein the values of R⁷ may be the same or different;
R¹⁷, R¹⁸, R¹⁹, R²³, R²⁵, R²⁹ or R³¹ are independently selected from halo, nitro, cyano, hydroxy, amino, carbamoyl, mercapto, sulphamoyl, hydroxyaminocarbonyl, amidino, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, (C₁₋₁₀alkyl)₃silyl, N—(C₁₋₁₀alkyl)amino, N,N—(C₁₋₁₀alkyl)₂amino, N,N,N—(C₁₋₁₀alkyl)₃ammonio, C₁₋₁₀ alkanoylamino, N—(C₁₋₁₀alkyl)carbamoyl, N,N—(C₁₋₁₀alkyl)₂carbamoyl, C₁₋₁₀alkylS(O)ₐ wherein a is 0 to 2, N—(C₁₋₁₀alkyl)sulphamoyl, N,N—(C₁₋₁₀alkyl)₂sulphamoyl, N—(C₁₋₁₀ alkyl)sulphamoylamino, N,N—(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R¹⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;
R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)- or —C(O)NR³⁶—; wherein R³⁶ is selected from hydrogen or C₁₋₆alkyl, and x is 0-2;
p, q, r and s are independently selected from 0-2;
R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, allyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;
R²⁰, R²⁴, R²⁶, R³⁰ or R³⁵ are independently selected from C₁₋₆alkyl, C₁₋₆alkanoyl, C₁₋₆ alkylsulphonyl, C₁₋₆ alkoxycarbonyl, carbamoyl, N—(C₁₋₆ alkyl)carbamoyl, N,N—(C₁₋₆ alkyl)carbamoyl, benzyl, benzyloxycarbonyl, benzoyl and phenylsulphonyl;
or a pharmaceutically acceptable salt, solvate or solvate of such a salt, or an in vivo hydrolysable ester formed on an available carboxy or hydroxy thereof, or an in vivo hydrolysable amide formed on an available carboxy thereof.

21. The method of claim 19, wherein the compound of Formula V is
1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxyethyl)carbamoyl]benzyl}carbamoylmethoxy)-2,3,4,5-tetrahydro-1,5-benzothiazepine;
or a pharmaceutically acceptable salt or solvate thereof.

22. The method of claim 1, wherein the dosage form comprises between 0.1 to 20 mg of the ASBTI.

23. The method of claim 1, wherein the dosage of the ASBTI is between about 0.5 mg and about 50 mg.

24. The method of claim 1, wherein the dosage of the ASBTI is any dosage from about 1 mg to about 20 mg.

25. The method of claim 1, wherein the dosage of the ASBTI is any dosage from about 1 mg to about 10 mg.

26. The method of claim 1, wherein the method reduces esophageal damage or necrosis.

27. The method of claim 1, wherein the method reduces intestinal metaplasia.

28. The method of claim 1, wherein the method reduces one or more symptoms selected from hematemesis, heartburn, regurgitation, dysphagia, odynophagia, nausea, weight loss, increased salivation, chest pain, reflux esophagitis, esophageal strictures, laryngitis, asthma, sinusitis, pharyngitis, globus pharingeus, globus hystericus, enamel erosion, and dentine hypersensitivity.

29. The method of claim 1, wherein the ASBTI is administered before ingestion of food, optionally wherein the ASBTI is administered less than about 60 minutes or less than about 30 minutes before ingestion of food.

30. The method of claim 1, wherein the ASBTI is administered orally.

31. The method of claim 1, wherein the ASBTI is administered as an ileal-pH sensitive release or an enterically coated formulation.

32. The method of claim 1, further comprising administration of a proton pump inhibitor, an H₂ antagonist, an H₂ receptor inhibitor, an antacid, a prokinetic, alginic acid, sucralfate, baclofen, or a combination thereof.

33. The method of claim 1, wherein further comprising a bile acid sequestrant or binder.
